(19) United States
(12) Patent Application Publication
(10) Pub. No.: US 2016/0244435 A1
(43) Pub. Date: Aug. 25, 2016

(54) EXPANDED THERAPEUTIC POTENTIAL IN NITROHETEROARYL ANTIMICROBIALS

(71) Applicants: The Regents of the University of California, Oakland, CA (US); THE SCRIPPS RESEARCH INSTITUTE, LA JOLLA, CA (US)

(72) Inventors: Lars Eckmann, San Diego, CA (US); Yukiko Miyamoto, San Diego, CA (US); Valery Fokin, Oceanside, CA (US); Karl Barry Sharpless, La Jolla, CA (US)

(73) Assignee: THE SCRIPPS RESEARCH INSTITUTE, La Jolla, CA (US)

(21) Appl. No.: 14/899,553

(22) PCT Filed: Jun. 20, 2014
(86) PCT No.: PCT/US14/43520

§ 371 Date: Dec. 17, 2015

Related U.S. Application Data
(60) Provisional application No. 61/838,122, filed on Jun. 21, 2013.

(51) Int. Cl. C07D 405/14; A61K 31/454; A61K 31/502; C07D 401/14; C07D 417/14; A61K 31/428; C07D 403/06; A61K 31/4192; A61K 45/06; C07D 403/14; A61K 31/55; A61K 31/4439; C07D 413/14; A61K 31/422; A61K 31/433; A61K 31/427; A61K 31/5377; A61K 31/506; A61K 31/4709; C07D 409/14; A61K 31/496

(52) U.S. Cl. CPC C07D 405/14; A61K 31/496; A61K 31/454; A61K 31/502; C07D 401/14; C07D 417/14; A61K 31/428; C07D 403/06; A61K 31/4192; A61K 45/06; C07D 403/14; A61K 31/55; A61K 31/4439; C07D 413/14; A61K 31/422; A61K 31/433; A61K 31/427; A61K 31/5377; A61K 31/506; A61K 31/4709; C07D 409/14

(57) ABSTRACT

Disclosed herein are antimicrobial compounds compositions, pharmaceutical compositions, the use and preparation thereof. Some embodiments relate to imidazole, thiazole, and furan derivatives and their use as therapeutic agents.

### INCORPORATION BY REFERENCE TO ANY PRIORITY APPLICATIONS

This application claims the benefit of U.S. Provisional Application No. 61/838,122, entitled Expanded Therapeutic Potential In Broad Activity Space Of Structurally Diverse Next-Generation 5-Nitroimidazole Antimicrobials, filed Jan. 21, 2013, the disclosure of which is incorporated herein by reference in its entirety.

### STATEMENT REGARDING FEDERALLY SPONSORED R&D

This invention was made with government support under AI075527 awarded by the National Institutes of Health, DK035108 awarded by the National Institutes of Health and DK080506 awarded by the National Institutes of Health. The government has certain rights in the invention.

### BACKGROUND OF THE INVENTION

### Field of the Invention

### Introduction

Antibiotics are among the greatest advances in medicine, yet their utility is constantly threatened by the development of resistance due to the high genetic adaptability of many target microbes. Most common antibiotics belong to a small number of functional and structural classes that target a limited set of microbial processes, including cell wall synthesis, protein translation, DNA replication, RNA transcription, and unique metabolic pathways. Despite these seemingly limited targeting opportunities, improved compounds have been developed within specific antibiotics classes over several drug generations with expanded potency and microbial range, as best illustrated by next-generation β-lactam antibiotics (1, 2).

Of particular importance among antibiotics are 5-nitro drugs, characterized by a nitro functional group in the 5-position of a five-membered heterocycle (imidazole, thiazole, or furan). The prototype and most commonly used drug of this class is the 5-nitroimidazole (5-NI) compound, metronidazole (Mz). Listed as an essential medicine by the WHO, it is one of the most versatile antibiotics in clinical practice, targeting a wide range of anaerobic microbes from protozoan parasites, including Giardia lamblia, Trichomononas vaginalis and Entamoeba histolytica to Gram-negative bacteria such as Helicobacter pylori, Clostridium difficile and Bacteroides fragilis (3, 4).

Mz and other 5-nitro antimicrobials are prodrugs that must be activated by reduction in the target microbe to generate toxic, short-lived radical intermediates. The radicals form adducts with different microbial molecules, including DNA, proteins and lipids, although the exact molecular targets and specific functional consequences are incompletely understood. The microbial specificity of 5-nitro drugs stems largely from the requirement for low redox potential electron transfers that do not occur in mammalian cells (5), although other, poorly defined aspects may also be important (6).

Antimicrobial therapy with Mz is usually effective, with reported success rates of 70-99%, depending on the specific infectious agent and patient population (7). However, Mz treatment failure and resistance occur for all target pathogens. For example, >50% of H. pylori cases are resistant to Mz in some developing countries (8). As many as 10-20% of patients with giardiasis show clinical resistance to Mz (4), while 2-4% of clinical T. vaginalis isolates display varying degrees of Mz resistance (9). In some cases, Mz resistance can be overcome by treatment with other 5-NI drugs, but many resistant microbial strains exhibit cross-resistance between the major available 5-NI drugs (10). Multiple mechanisms have been implicated in 5-NI drug resistance, including a diminished capacity to reduce and activate 5-nitro prodrugs (11-13) and detoxification of nitro drug radicals (14).

The common 5-NI drugs have different simple side chains at the 1-position of the imidazole, e.g. Mz possesses a hydroxyethyl group while tinidazole has an ethylsulfonylethyl group. These modifications mostly affect the pharmacokinetic properties of the drugs, but have only limited influence on drug potency or ability to overcome resistance (10). However, other structural modifications of 5-NI compounds can improve antimicrobial activity and resistance profiles (6, 15) or confer new antimicrobial activities, as shown for the kinetoplastid Trypanosoma cruzi (16). Despite these promising observations, the full antimicrobial potential of 5-NI drugs is not known, partly because commercial drug development largely ceased after approval of the first-generation 5-NI drugs beginning in the 1960s. Concerns about long-term safety may have contributed to this situation, but extensive clinical studies have shown that these compounds are safe and have no relevant long-term toxicity in humans (17).

### SUMMARY OF THE INVENTION

One embodiment relates to a compound having the structure of Formula (I)

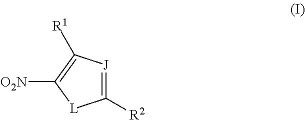

or prodrug or pharmaceutically acceptable salts thereof, wherein
J is N or CR³;
L is NR⁴, S, or O;
R¹ is hydrogen or —(CH₂)ₘ—Y—R⁵;
R² is hydrogen, —(CH₂)ₘ—Y—R⁵, —CH═CH—R⁵, —CHR^{a}—CHR^{b}—R⁵, —C═N—O—(CH₂)ₘ—R⁵, —NHC(O)—(CH₂)ₘ—R⁵, optionally substituted —S—C₁₋₆ alkyl, optionally substituted —O—C₁₋₆alkyl, optionally substituted C₁₋₆alkyl, optionally substituted C₂₋₁₀alkenyl, optionally substituted C₂₋₁₀alkynyl, optionally substituted C₃₋₁₀ cycloalkyl, optionally substituted 4-10 membered heterocyclyl, optionally substituted C₆₋₁₀ aryl, or optionally substituted 5-10 membered heteroaryl;
R³ is hydrogen, halogen, hydroxyl, or optionally substituted —C₁₋₆ alkyl;
R⁴ is hydrogen, C₁₋₆alkyl or —(CH₂)ₘ—Y—R⁵;
m is an integer between 0 to 5;
Y is optionally substituted heteroaryl;
each R⁵ is independently hydrogen, —(CH₂)ₙ-M, or —(CH₂)ₙ—Y′-M;
each M is independently —C(O)M′, —C(O)O—C₁₋₄alkyl, —S(O)₂-M′, —S-M′, —NHC(O)-M′, —NHC(O)NH-M′, —O—C₆H₄—C(O)NH-M′, —O-M′, —O(CH₂)C(O)-M′, —N(CH₃)-M′, optionally substituted —S—C₁₋₆ alkyl, optionally substituted —O—C₁₋₆alkyl, optionally substituted C₁₋₆alkyl, optionally substituted C₂₋₁₀alkenyl, optionally substituted C₂₋₁₀alkynyl, optionally substituted C₃₋₁₀ cycloalkyl, optionally substituted 4-10 membered heterocyclyl, optionally substituted C₆₋₁₀ aryl, or optionally substituted 5-13 membered heteroaryl;
each M′ is independently hydrogen, optionally substituted C₁₋₆alkyl, optionally substituted C₂₋₁₀alkenyl, optionally substituted C₂₋₁₀alkynyl, optionally substituted C₃₋₁₀ cycloalkyl, optionally substituted 4-10 membered heterocyclyl, optionally substituted C₆₋₁₀ aryl, or optionally substituted 5-13 membered heteroaryl;
each Y′ is independently optionally substituted 4-10 membered heterocyclyl or C₆₋₁₀ aryl;
n is an integer between 0 to 5;
R^{a} and R^{b} are each independently selected from —H, hydroxy, halogen, —C—O—C₁₋₄alkyl, —O—C(O)—C₁₋₄alkyl;
with the proviso that the compound does not have the structure selected from the group consisting of

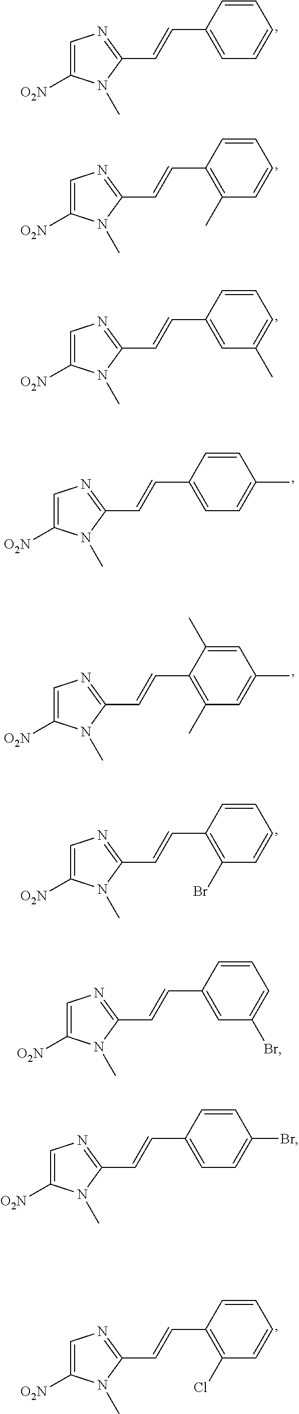

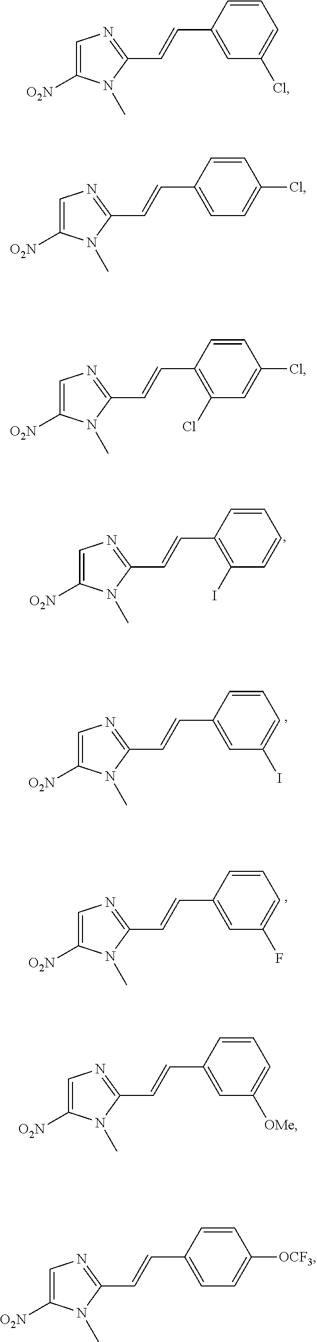

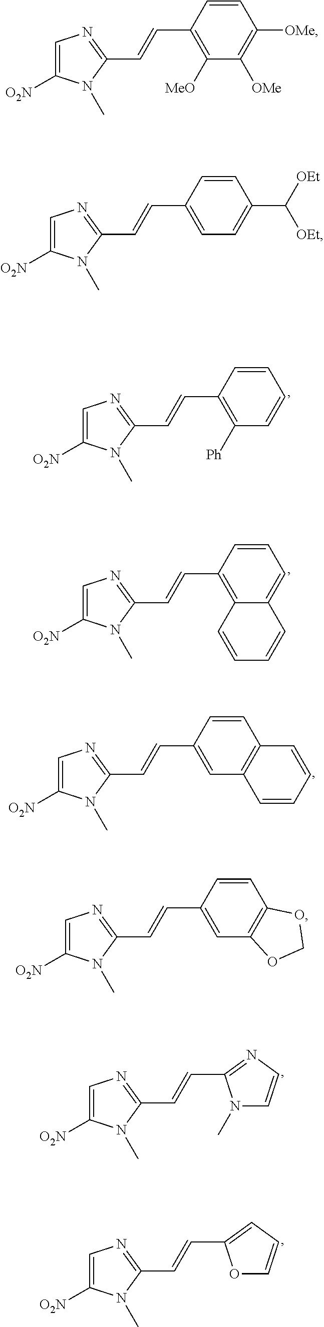

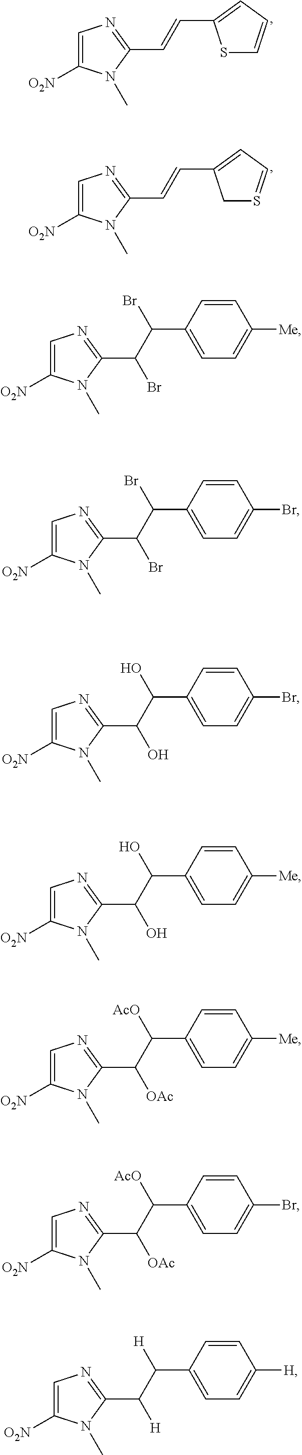

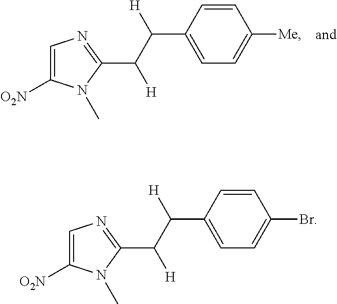 [SMILES: Cc1ccc(CCc2ncc([N+](=O)[O-])n2C)cc1 ; "and" ; Cn1c([N+](=O)[O-])cnc1CCc1ccc(Br)cc1]

It will be appreciated that any of the possible identifiers of J may be combined with any of the possible identifiers for J, L, R¹, R², R³, R⁴, R⁵, R^{a}, R^{b}, Y′, and M′.

It will be appreciated that any of the possible identifiers of L may be combined with any of the possible identifiers for J, L, R¹, R², R³, R⁴, R⁵, R^{a}, R^{b}, Y, M, Y′, and M′.

It will be appreciated that any of the possible identifiers of R¹ may be combined with any of the possible identifiers for J, L, R¹, R², R³, R⁴, R⁵, R^{a}, R^{b}, Y, M, Y′, and M′.

It will be appreciated that any of the possible identifiers of R² may be combined with any of the possible identifiers for J, L, R¹, R², R², R⁴, R^{a}, R^{b}, Y, M, Y′, and M′.

It will be appreciated that any of the possible identifiers of R³ may be combined with any of the possible identifiers for J, L, R¹, R², R², R⁴, R^{a}, R^{b}, Y, M, Y′, and M′.

It will be appreciated that any of the possible identifiers of R⁴ may be combined with any of the possible identifiers for J, L, R¹, R², R², R⁴, R^{a}, R^{b}, Y, M, Y′, and M′.

It will be appreciated that any of the possible identifiers of R⁵ may be combined with any of the possible identifiers for J, L, R¹, R², R², R⁴, R^{a}, R^{b}, Y, M, Y′, and M′.

It will be appreciated that any of the possible identifiers of R^{a} may be combined with any of the possible identifiers for J, L, R¹, R², R², R⁴, R^{a}, R^{b}, Y, M, Y′, and M′.

It will be appreciated that any of the possible identifiers of R^{b} may be combined with any of the possible identifiers for J, L, R¹, R², R², R⁴, R^{a}, R^{b}, Y, M, Y′, and M′.

It will be appreciated that any of the possible identifiers of Y may be combined with any of the possible identifiers for J, L, R¹, R², R², R⁴, R^{a}, R^{b}, Y, M, Y′, and M′.

It will be appreciated that any of the possible identifiers of M may be combined with any of the possible identifiers for J, L, R¹, R², R², R⁴, R^{a}, R^{b}, Y, M, Y′, and M′.

It will be appreciated that any of the possible identifiers of Y′ may be combined with any of the possible identifiers for J, L, R¹, R², R², R⁴, R^{a}, R^{b}, Y, M, Y′, and M′.

It will be appreciated that any of the possible identifiers of M′ may be combined with any of the possible identifiers for J, L, R¹, R², R², R⁴, R^{a}, R^{b}, Y, M, Y′, and M′.

One embodiment is a pharmaceutical composition comprising an agent and a pharmaceutically acceptable carrier, wherein the agent is a compound from Table 1.3 or a pharmaceutically acceptable salt or prodrug thereof.

Another embodiment is a pharmaceutical composition comprising an agent and a pharmaceutically acceptable carrier, wherein the agent is a compound from Table S1a or a pharmaceutically acceptable salt or prodrug thereof.

One embodiment is a pharmaceutical composition comprising an agent and a pharmaceutically acceptable carrier, wherein the agent is a compound from Table 1.3 or a pharmaceutically acceptable salt or prodrug thereof for use in the treatment of an infection in an individual.

Another embodiment is a pharmaceutical composition comprising an agent and a pharmaceutically acceptable carrier, wherein the agent is a compound from Table S1a or a pharmaceutically acceptable salt or prodrug thereof for use in the treatment of an infection in an individual.

One embodiment is a pill comprising a compound from Table 1.3 or a pharmaceutically acceptable salt or prodrug thereof.

One embodiment is a pill comprising a compound from Table S1a or a pharmaceutically acceptable salt or prodrug thereof.

One embodiment is a method of treating a giardiasis infection in an individual in need thereof comprising administering a pharmaceutical composition comprising an agent and a pharmaceutically acceptable carrier, wherein the agent is a compound from Table S1a or a pharmaceutically acceptable salt or prodrug thereof to said individual.

One embodiment is a compound of Formula (II)

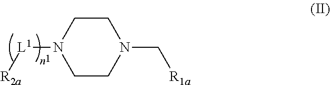

Wherein R₁ₐ is a C₂-C₆ alkyne; L¹ is a CH₂, C═O, or S═O; R₂ₐ is an optionally substituted arylene, an optionally substituted heteroarylene, an optionally substituted C₃-C₈ cycloalkyl, or OCH₂CH₃; and n¹ is 0 or 1.

In some embodiments, R₁ₐ is a —C≡CH

In some embodiments, R₁ₐ is a

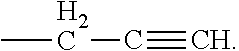

In some embodiments, R₂ₐ is optionally substituted phenyl; optionally substituted furanyl, optionally substituted, optionally substituted pyridine, optionally substituted benzodioxole.

One embodiment is compound having the structure selected from Table 1.3

One embodiment is a pharmaceutical composition comprising an agent and a pharmaceutically acceptable carrier, wherein the agent is a compound having the formula:

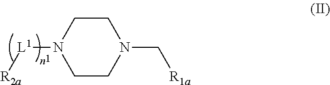

Wherein R₁ₐ is a C₂-C₆ alkyne; L¹ is a CH₂, C═O, or S═O; R₂ₐ is an optionally substituted arylene, an optionally substituted heteroarylene, an optionally substituted C₃-C₈ cycloalkyl, or OCH₂CH₃; and n is 0 or 1.

### BRIEF DESCRIPTION OF THE DRAWINGS

FIG. 1 shows the synthesis of comprehensive new 5-NI library. FIG. 1A shows the structure of metronidazole (Mz). FIG. 1B shows the that six different 5-NI cores (A-F) were synthesized with a “clickable” azide (N₃) functional group. FIG. 1C shows the scheme of click chemistry-facilitated synthesis of 5-NI triazole library.

FIG. 2 shows the expanded antimicrobial activity range of new 5-NI compounds. FIG. 2A shows that the activities of 378 compounds were tested against the indicated protozoa and bacteria in 24-48 h growth assays using ATP levels or OD600 as read-outs; FIG. 2B lists examples of compounds with enhanced broad-spectrum or pathogen-selective activity (key values in bold); FIG. 2C displays relationships between activities of individual compounds against the four target pathogens; FIG. 2D shows the relationship between compound activities against two colonic bacteria.

FIG. 3 shows non-limiting examples of new 5-NI compounds which overcome Mz resistance. FIG. 3A shows the results when compounds were tested against MzR strains of G. lamblia and T. vaginalis; FIG. 3B shows activities against wild-type and ΔfrxA ΔrdxA strains of H. pylori.

FIG. 4 shows non-limiting examples of the bioactivity landscape of 5-NI compounds. FIG. 4A shows that the individual compounds were plotted in the resulting space with the top 10% most potent broad-spectrum compounds shown as dark circles; FIG. 4B shows that a structural space was constructed from the activity data against MzR G. lamblia; FIG. 4C shows a service vector machine model which was constructed from the activity data of the 378 compounds against MzR Giardia (training set), and applied prospectively to a new set of 281 independently synthesized 5-NI compounds (test set).

FIG. 5 shows Structure-activity relationships of 5-NI building blocks. FIG. 5A A shows the influence of the azido-5-NI cores A to F on activity against Mz-sensitive (MzS, left) and Mz-resistant (MzR, right) Giardia, with average activities shown as the darklines; FIG. 5B depicts data of all compounds generated from cores A-C (to minimize core bias in the alkyne evaluation) in a structural space derived by principal component analysis.

FIG. 6 shows In vivo efficacy of 5-NI compounds against giardiasis. FIG. 6A shows the trophozoite numbers in the small intestine; FIG. 6B shows the relationships of in vivo bioactivity, in vitro activity, aqueous solubility, and measured serum drug concentrations are shown in FIG. 6B; FIG. 6C shows examples for in vivo active compounds along with their in vitro activities against MzS and MzR Giardia.

FIG. 7 shows the core structures and synthesis of new nitro drug library. FIG. 7A shows the library of new nitro compounds; and FIG. 7B shows the activities of these compounds against T. vaginalis F1623.

FIG. 8 shows the antigiardial activity of some nitro compounds. FIG. 8A shows the activity of all library compounds was tested against the Mz-sensitive (MzS) G. lamblia strain 713. FIG. 8B shows a subset of compounds was tested against a second Giardia lines, 106, and the data were related to those in the 713 line.

FIG. 9 shows the drug activity against MzR Giardia. FIG. 9A shows activities of 180 selected nitro compounds which were determined against MzR lines of G. lamblia 713 and 106, and are expressed as percentages of residual activity (RA) relative to the parental MzS cells; FIG. 9B shows detailed information on their antigiardial activities and structures.

FIG. 10 shows the in vivo efficacy of some nitro-compounds. FIG. 10A shows the results when live trophozoites of adult C57BL/6 mice infected orally with G. lamblia GS/M after two-day treatment; FIG. 10B shows structures for two effective compounds. FIG. 10 C shows that gerbils were infected and treated with different single oral drug doses; FIG. 10 D displays the results when plasma levels of several representative active and inactive compounds at 2 h after a single oral dose.

FIG. 11 shows a non-limiting example of the drug testing and development strategy.

FIG. 12 shows the nitrodrug activities against periodontal bacteria.

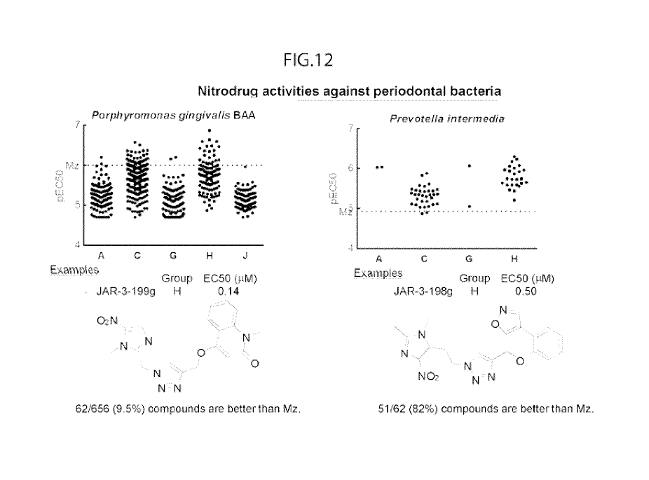

FIG. 12A shows activities against porphyromonas gingivalis bacteria; FIG. 12B shows activities against prevotella intermedia; FIG. 12C shows activities against Fusobacterium nucleatum; and FIG. 12D shows activities against T. forsythia.

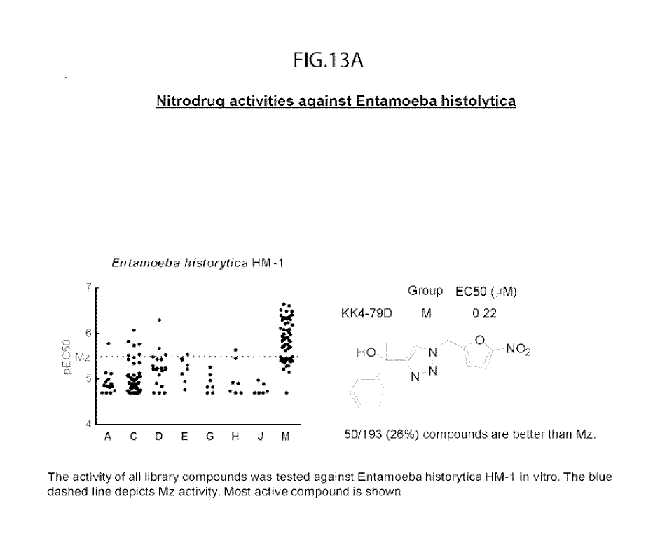

FIG. 13 shows the activities of nitro compounds against Entamoeba histolytica HM-1 in vitro.

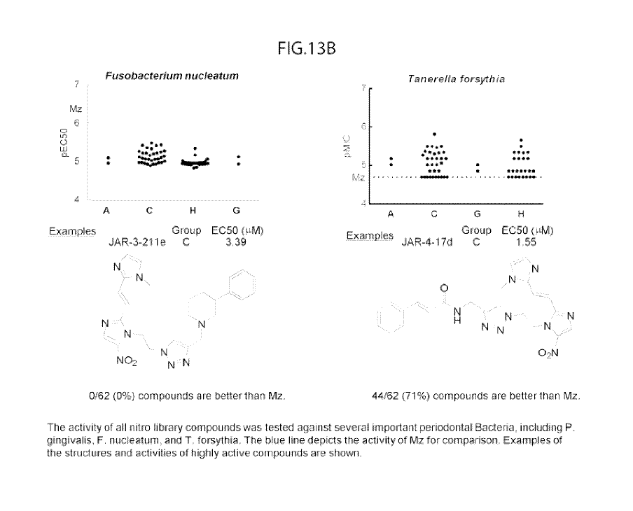

FIG. 14 shows the nitro drug activities against mycobacterium tuberculosis H37R in vitro.

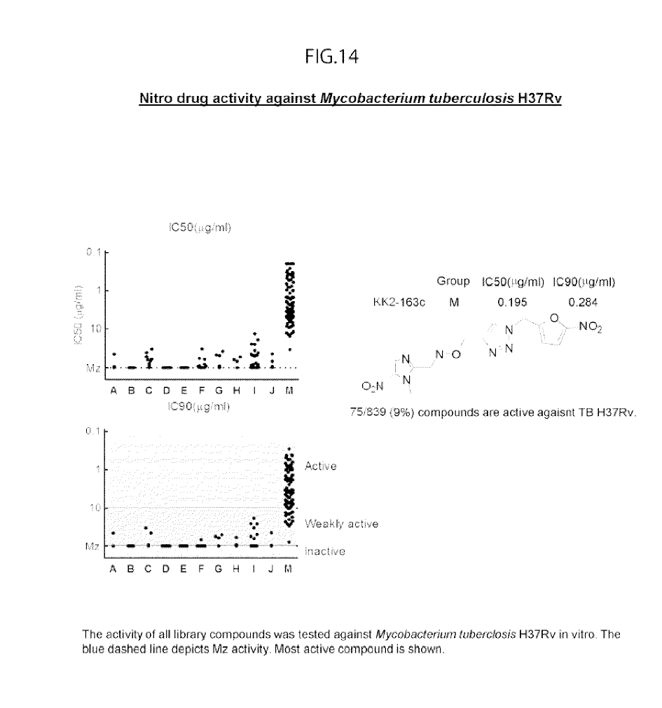

FIG. 15 shows an example of the expanded antimicrobial activity range of new 5-NI compounds. FIG. 15 A shows the activities of nitro compounds which were tested against H. pylori (strain SS1) and FIG. 15 B shows the activities of nitro compound tested against G. lamblia (strain 713).

FIG. 16 shows the relationship of compound activities between clinical isolates. FIG. 16 A shows the 5-NI library was tested for activity against two different clinical Mz-sensitive (MzS) isolates of G. lamblia (strains 713 and 106); FIG. 16B shows the results when the 5-NI library was tested for activity against T. vaginalis (strains G3 and F1623); and FIG. 16C shows the results when the 5-NI library was tested for activity against and H. pylori (strains SS1 and CS22).

FIG. 17 shows the distribution of active 5-NI compounds in chemical space.

FIG. 18 shows the structural analysis of alkyne sets used in library generation.

FIG. 19 shows the structural analysis of in vivo active 5-NI compounds.

FIG. 20 shows the chemical descriptors for prediction of in vivo activity.

### DETAILED DESCRIPTION OF THE PREFERRED EMBODIMENT

The present disclosure relates to a large library of structurally diverse 5-NI compounds for comprehensive and unbiased evaluation of the therapeutic potential of this important class of antimicrobials. Using a new approach to 5-NI synthesis, we show here that many of the >650 new 5-NI compounds have vastly improved activity against a range of microbial targets, as they display marked improvements in broad-spectrum activity, can overcome different forms of 5-NI drug resistance, and are active and non-toxic in animal infection models. These findings substantially broaden the potential structural space suitable for further development of improved nitro antimicrobials for ultimate clinical use, and strongly suggest that the systematic development of next-generation 5-NI drugs can lead to new agents in the armamentarium of antimicrobials against a wide range of clinically important infections.

In some embodiments, the compounds have the structure of Formula (I) with described herein has the proviso that when L is —N—CH₃ and J is N, R² is not —CH═CH—R^{e} or —CHX—XHY—R^{f}, wherein R^{e} is optionally substituted phenyl, naphthalenyl, thiophene, furan, 1,3-benzodioxol, or imidazolinylmethyl; and R^{f} is phenyl optionally substituted at the para position with methyl or bromine.

In some embodiments, for the compound described herein, J is N and L is NR⁴.

In some embodiments, the compound of Formula (I) described herein has the structure of Formula (I-A)

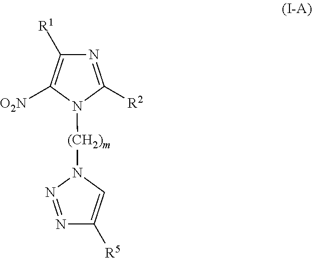

or prodrug or pharmaceutically acceptable salts thereof, wherein R¹, R², and R⁵ have the identifiers set forth in Formula (I).

In some embodiments, the compound of Formula (I) or I(A) is selected from compounds A-101 to A-163 in Table S1a and A-201- to A-247 in Table S6a.

In some embodiments, the compound of Formula (I) described herein has the structure of Formula (I-B)

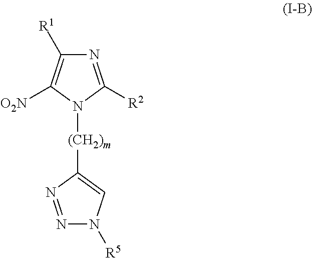

or prodrug or pharmaceutically acceptable salts thereof, wherein m is an integer in the range of 1 to 5, and wherein R¹, R², and R⁵ have the identifiers set forth in Formula (I).

In some embodiments, the compound of Formula (I) or 1(B) is selected from the group consisting of compounds A-101 to A-163 in Table S1a and A-201- to A-247 in Table S6a.

In some embodiments, R² is C₁₋₆ alkyl.

In some embodiments, R² is methyl.

In some embodiments, the compound described herein has the structure of Formula (I-C)

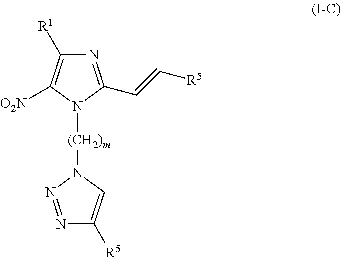

or prodrug or pharmaceutically acceptable salts thereof, wherein Wand R⁵ have the identifiers set forth in Formula (I).

In some embodiments, the compound of Formula (I) or Formula (I-C) is selected from compounds C(1)-101 to C(1)-163 in Table S1e, C(2)-101 to C(2)-163 in Table S1e, C(1)-201- to C(1)-247 in Table S6e, and C(2)-201- to C(2)-247 in Table S6e.

In some embodiments, m is 2.

In some embodiments, m is 1.

In some embodiments, m is 3.

In some embodiments, m is 4.

In some embodiments, m is 5.

In some embodiments, the compound described herein has the structure of Formula (I-D)

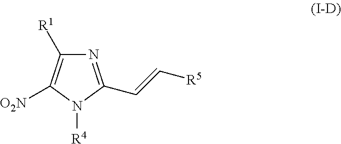

or prodrug or pharmaceutically acceptable salts thereof, wherein R¹, R⁴, and R⁵ have the identifiers set forth in Formula (I).

In some embodiments, the compound described herein has the structure of Formula (I-E)

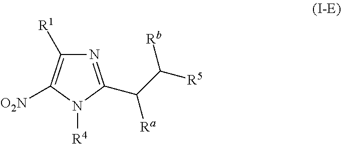

or prodrug or pharmaceutically acceptable salts thereof, wherein R¹, R⁴, R^{a}, and R⁵ have the identifiers set forth in Formula (I).

In some embodiments, the compound described herein has the structure of Formula (I-F)

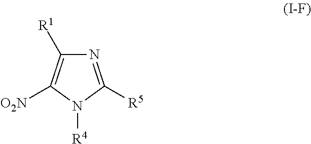

or prodrug or pharmaceutically acceptable salts thereof, wherein R¹, R⁴, and R⁵ have the identifiers set forth in Formula (I).

In some embodiments, the compound of Formula (I-F) is selected from compounds in Table S10.

In some embodiments, the compound described herein has the structure of Formula (I-G)

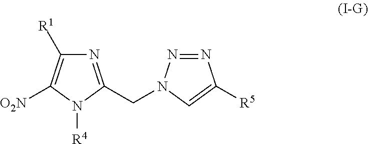

or prodrug or pharmaceutically acceptable salts thereof, wherein R¹, R⁴, and R⁵ have the identifiers set forth in Formula (I).

In some embodiments, the compound of Formula (I) or (I-G) is selected from compounds G-101 to G-163 in Table S1b and G-201- to G-247 in Table S6b.

In some embodiments, the compound described herein has the structure of Formula (I-H)

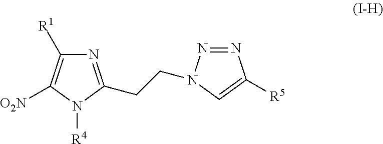

or prodrug or pharmaceutically acceptable salts thereof, wherein R¹, R⁴, and R⁵ have the identifiers set forth in Formula (I).

In some embodiments, the compound of formula (I) or (I-H) is selected from compounds H-101 to H-163 in Table S1c and H-201- to H-247 in Table S6c.

In some embodiments, the compound described herein has the structure of Formula (I-I)

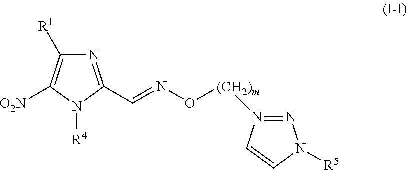

or prodrug or pharmaceutically acceptable salts thereof, wherein R¹, R⁴, and R⁵ have the identifiers set forth in Formula (I).

In some embodiments, the compound of Formula (I-I) is selected from compounds in Table S11.

In some embodiments, the compound described herein has the structure of Formula (I-J)

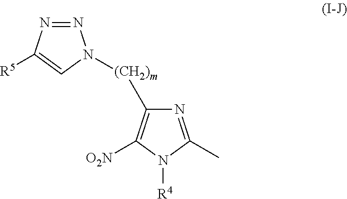

or prodrug or pharmaceutically acceptable salts thereof, wherein R⁴ and R⁵ have the identifiers set forth in Formula (I).

In some embodiments, the compound of (I) or (I-J) is selected from compounds J-101 to J-163 in Table S1d and J-201- to J-247 in Table S6d.

In some embodiments, R⁴ is hydrogen or C₁₋₄alkyl. In some embodiments, R⁴ is methyl.

In some embodiments, the compound described herein has the structure of Formula (I-K)

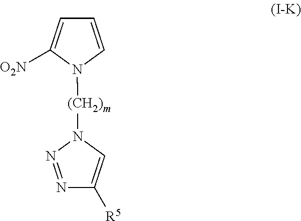

or prodrug or pharmaceutically acceptable salts thereof, wherein R⁵ have the identifiers set forth in Formula (I).

In some embodiments, the compound described herein has the structure of Formula (I-L)

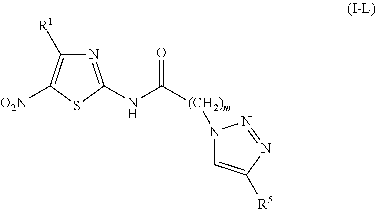

or prodrug or pharmaceutically acceptable salts thereof, wherein R¹ and R⁵ have the identifiers set forth in Formula (I).

In some embodiments, the compound of Formula (I-L) is selected from compounds in Table S12.

In some embodiments, the compound described herein has the structure of Formula (I-M)

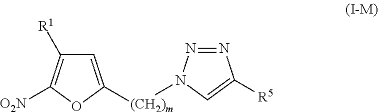

or prodrug or pharmaceutically acceptable salts thereof, wherein R′ and R³ have the identifiers set forth in Formula (I).

In some embodiments, the compound of Formula (I-M) is selected from compounds in Table S13.

In some embodiments, R¹ is hydrogen.

In some embodiments, R¹ is methyl.

In some embodiments, R⁵ is selected from the group consisting of

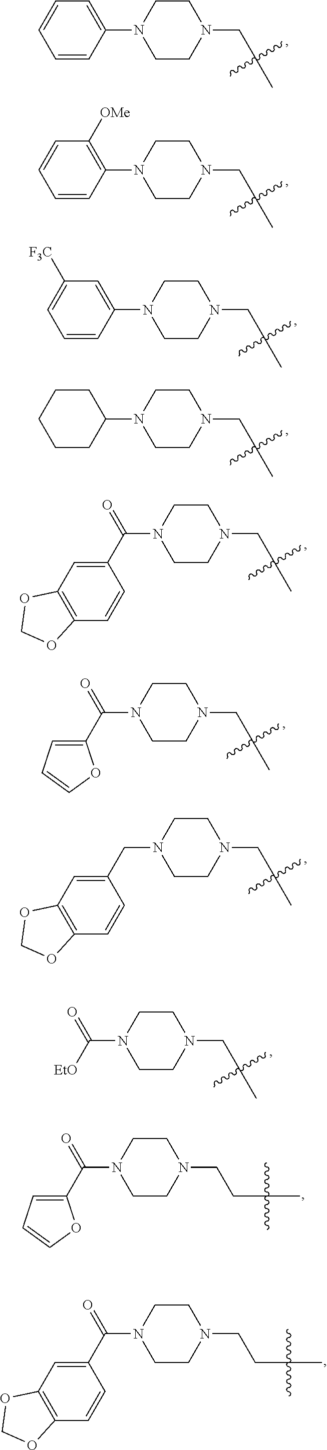

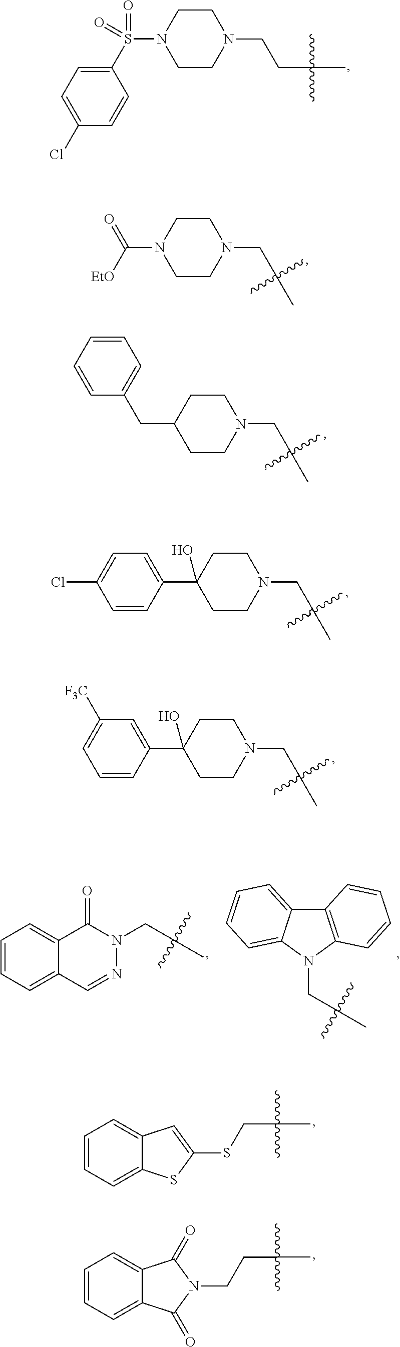

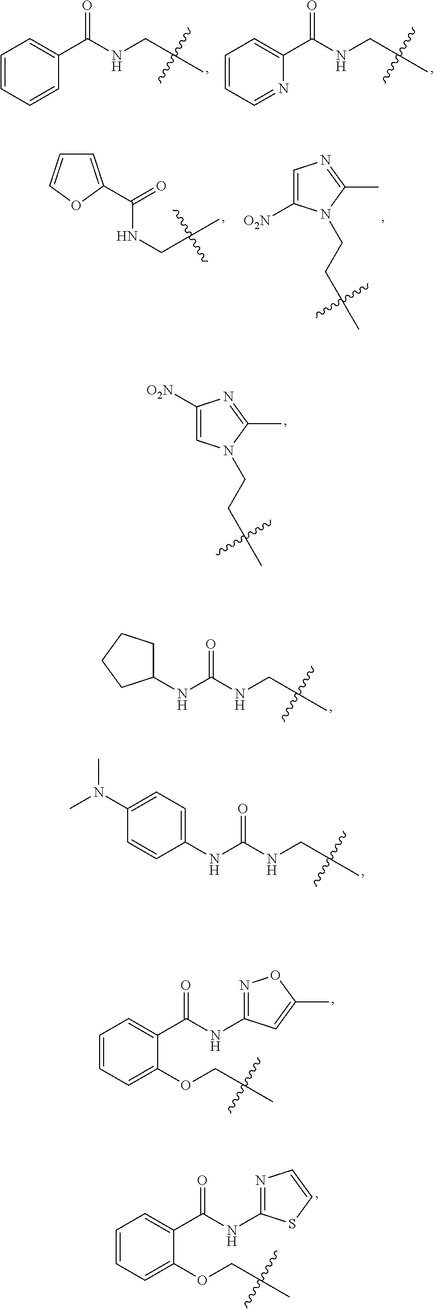

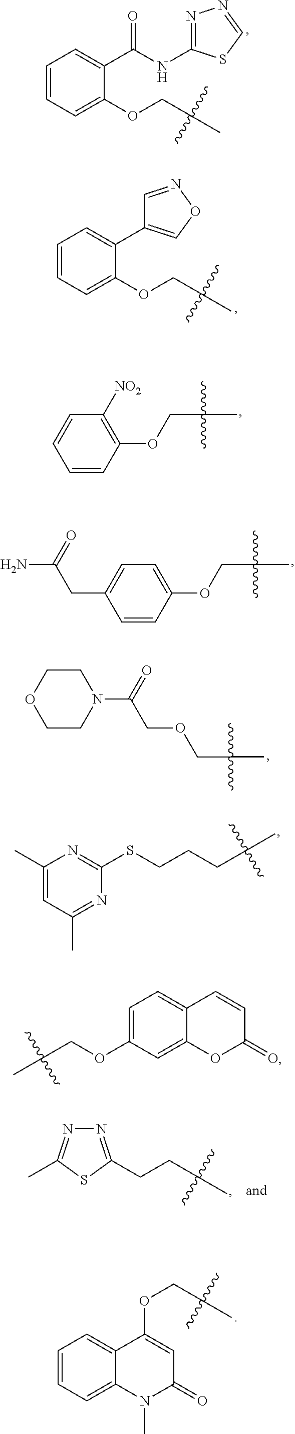

In some embodiments, R⁵ is selected from the group consisting of

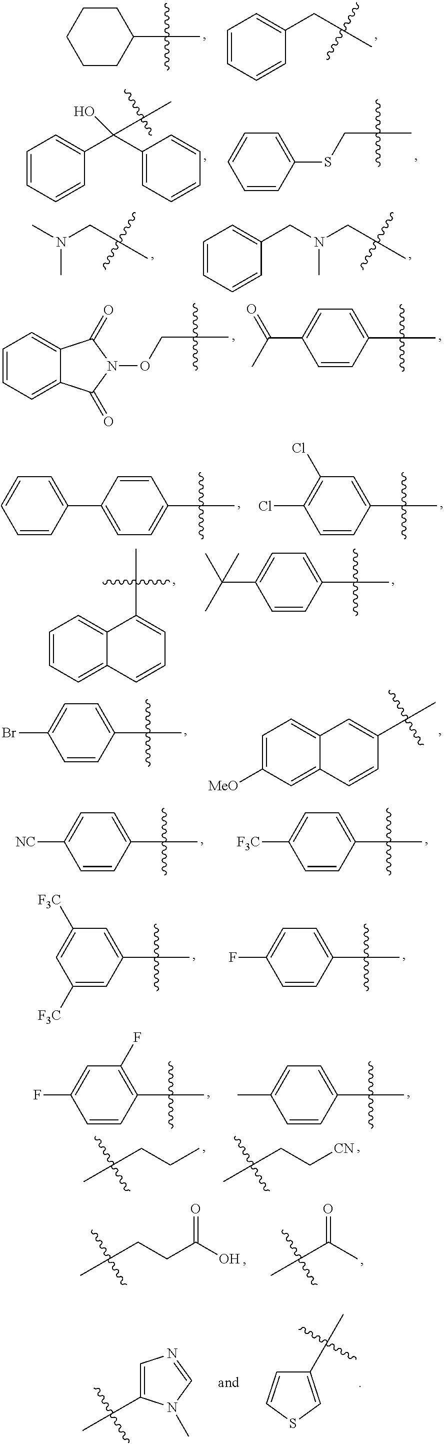

In some embodiments, R⁵ is selected from the group consisting of

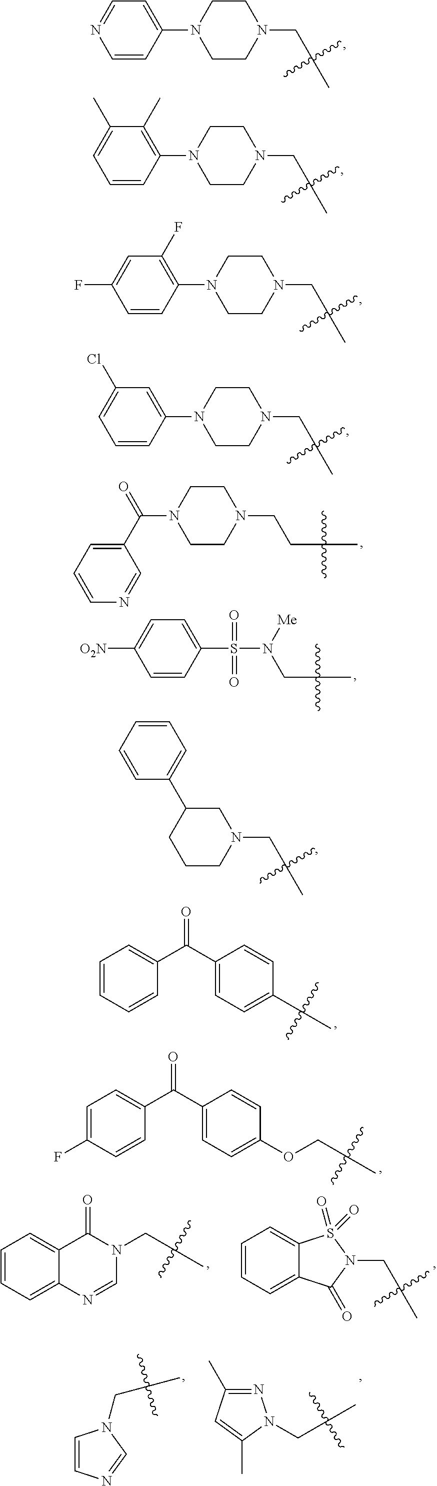

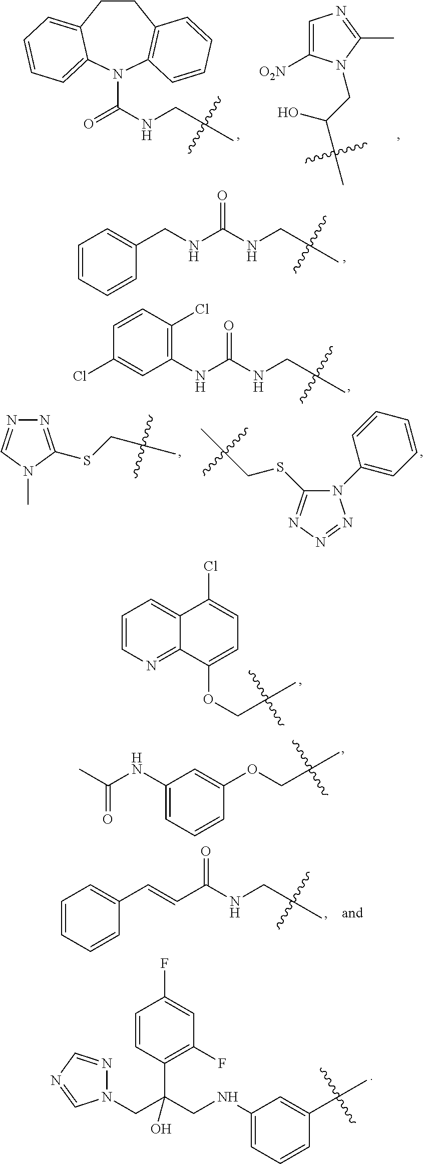

In some embodiments, R⁵ is selected from the group consisting of

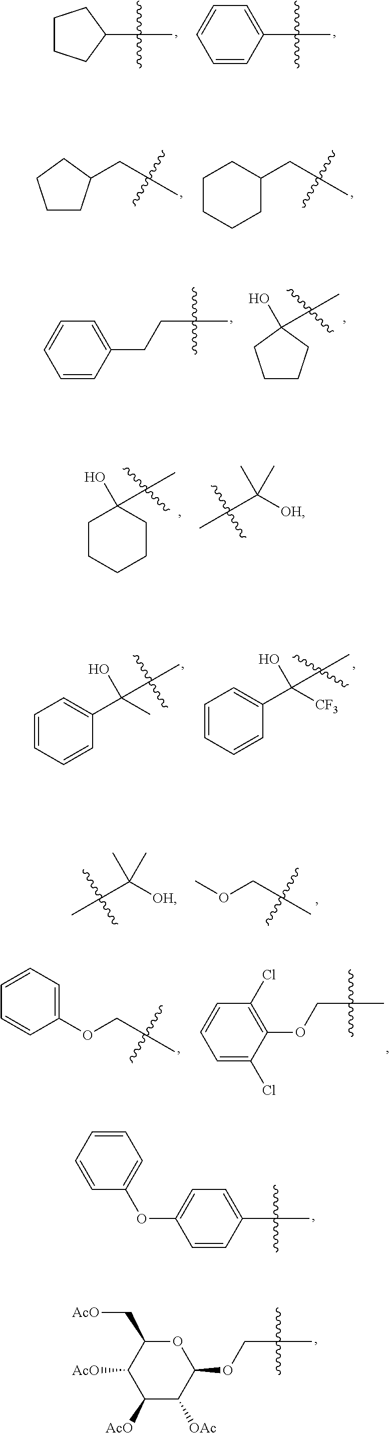

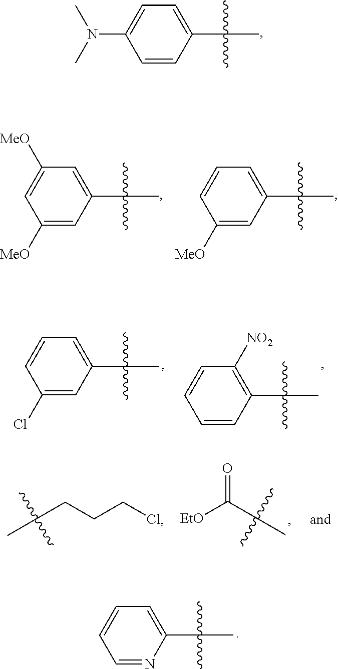

Some embodiments relate to a pharmaceutical composition comprising a therapeutically effective amount of a compound described herein and a pharmaceutically acceptable excipient.

Some embodiments relate to the pharmaceutical composition described herein further includes an additional medicament.

In some embodiments, the additional medicament is selected from an antibacterial agent, an antifungal agent, an antiviral agent, an anti-inflammatory agent, or an anti-allergic agent.

Some embodiments relate to a method of ameliorating a Trichomononas vaginalis infection, comprising administering to a subject in need thereof using a therapeutically effective amount of a compound described herein.

In some embodiments, the method further includes administering to the subject an additional medicament.

In some embodiments, the additional medicament is selected from an antibacterial agent, an antifungal agent, an antiviral agent, an anti-inflammatory agent, or an antiallergic agent.

Some embodiments relate to a method of ameliorating a Giardia lamblia infection, comprising administering to a subject in need thereof using a therapeutically effective amount of a compound described herein.

Some embodiments relate to a method of ameliorating a Entamoeba histolytica infection, comprising administering to a subject in need thereof a therapeutically effective amount of a compound described herein.

Some embodiments relate to a method of ameliorating a Gram-negative bacterial infection, comprising administering to a subject in need thereof a therapeutically effective amount of a compound described herein.

In some embodiments, the gram-negative bacteria is selected from Helicobacter pylori, Clostridium difficile and Bacteroides fragilis.

In some embodiments, the subject is a mammal.

In some embodiments, the mammal is a human.

Some embodiments relate to use of the compound described herein for ameliorating a Trichomononas vaginalis infection.

Some embodiments relate to use of the compound described herein for ameliorating a Giardia lamblia infection.

Some embodiments relate to use of the compound described herein for ameliorating a Entamoeba histolytica infection.

### DEFINITIONS

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference in its entirety.

The term “pro-drug ester” refers to derivatives of the compounds disclosed herein formed by the addition of any of several ester-forming groups that are hydrolyzed under physiological conditions. Examples of pro-drug ester groups include pivoyloxymethyl, acetoxymethyl, phthalidyl, indanyl and methoxymethyl, as well as other such groups known in the art, including a (5-R-2-oxo-1,3-dioxolen-4-yl)methyl group. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is herein incorporated by reference in their entirety.

“Metabolites” of the compounds disclosed herein include active species that are produced upon introduction of the compounds into the biological milieu.

“Solvate” refers to the compound formed by the interaction of a solvent and a compound described herein, a metabolite, or salt thereof. Suitable solvates are pharmaceutically acceptable solvates including hydrates.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group of the compounds may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

Some embodiments relate to use of the compound described herein for ameliorating a Gram-negative bacterial infection.

The term “alkyl” refers to a straight or branched monovalent hydrocarbon containing, unless otherwise stated, 1-20 carbon atoms (e.g., C₁-C₁₀). Examples of alkyl include, but are not limited to, methyl, ethyl, n-propyl, i-propyl, n-butyl, i-butyl, and I-butyl. The term “alkenyl” refers to a straight or branched monovalent or bivalent hydrocarbon containing 2-20 carbon atoms (e.g., C₂-C₁₀) and one or more double bonds. Examples of alkenyl include, but are not limited to, ethenyl, propenyl, propenylene, allyl, and 1,4-butadienyl. The term “alkynyl” refers to a straight or branched monovalent or bivalent hydrocarbon containing 2-20 carbon atoms (e.g., C₂-C₁₀) and one or more triple bonds. Examples of alkynyl include, but are not limited to, ethynyl, ethynylene, 1-propynyl, 1- and 2-butynyl, and 1-methyl-2-butynyl. The term “alkoxy” refers to an —O-alkyl radical. Examples of alkoxy include, but are not limited to, methoxy, ethoxy, n-propoxy, isopropoxy, n-butoxy, iso-butoxy, sec-butoxy, and tert-butoxy. The term “acyloxy” refers to an —O—C(O)—R radical in which R can be H, alkyl, alkenyl, alkynyl, cycloalkyl, cycloalkenyl, heterocycloalkyl, heterocycloalkenyl, aryl, or heteroaryl.

The term “cycloalkyl” refers to a monovalent or bivalent saturated hydrocarbon ring system having 3 to 30 carbon atoms (e.g., C₃-C₁₂). Examples of cycloalkyl include, but are not limited to, cyclopropyl, cyclobutyl, cyclopentyl, cyclohexyl, 1,4-cyclohexylene, cycloheptyl, cyclooctyl, and adamantine. The term “cycloalkenyl” refers to a monovalent or bivalent non-aromatic hydrocarbon ring system having 3 to 30 carbons (e.g., C₃-C₁₂) and one or more double bonds. Examples include cyclopentenyl, cyclohexenyl, and cycloheptenyl. The term “heterocycloalkyl” refers to a monovalent or bivalent nonaromatic 5-8 membered monocyclic, 8-12 membered bicyclic, or 11-14 membered tricyclic ring system having one or more heteroatoms (such as O, N, S, or Se). Examples of heterocycloalkyl groups include, but are not limited to, piperazinyl, pyrrolidinyl, dioxanyl, morpholinyl, and tetrahydrofuranyl. The term “heterocycloalkenyl” refers to a monovalent or bivalent nonaromatic 5-8 membered monocyclic, 8-12 membered bicyclic, or 11-14 membered tricyclic ring system having one or more heteroatoms (such as O, N, S, or Se) and one or more double bonds.

The term “aryl” refers to a monovalent 6-carbon monocyclic, 10-carbon bicyclic, 14-carbon tricyclic aromatic ring system. Examples of aryl groups include, but are not limited to, phenyl, naphthyl, and anthracenyl. The term “arylene” refers to a bivalent 6-carbon monocyclic, 10-carbon bicyclic, 14-carbon tricyclic aromatic ring system. The term “aryloxyl” refers to an —O-aryl. The term “arylamino” refers to an —N(R)-aryl in which R can be H, alkyl, alkenyl, alkynyl, cycloalkyl, cycloalkenyl, heterocycloalkyl, heterocycloalkenyl, aryl, or heteroaryl. The term “heteroaryl” refers to a monvalent aromatic 5-8 membered monocyclic, 8-12 membered bicyclic, or 11-14 membered tricyclic ring system having one or more heteroatoms (such as O, N, S, or Se). Examples of heteroaryl groups include pyridyl, furyl, imidazolyl, benzimidazolyl, pyrimidinyl, thienyl, quinolinyl, indolyl, and thiazolyl. The term “heteroarylene” refers to a bivalent aromatic 5-8 membered monocyclic, 8-12 membered bicyclic, or 11-14 membered tricyclic ring system having one or more heteroatoms (such as O, N, S, or Se).

Optionally substituted alkyl, optionally substituted alkenyl, optionally substituted alkynyl, optionally substituted cycloalkyl, optionally substituted heterocycloalkyl, optionally substituted cycloalkenyl, optionally substituted heterocycloalkenyl, optionally substituted aryl, and optionally substituted heteroaryl mentioned above include both substituted and unsubstituted moieties. Possible substituents on amino, cycloalkyl, heterocycloalkyl, cycloalkenyl, heterocycloalkenyl, aryl, arylene, heteroaryl, and heteroarylene include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, arylamino, hydroxy, halo, oxo (O═), thioxo (S═), thio, silyl, C₁-C₁₀ alkylthio, arylthio, C₁-C₁₀ alkylsulfonyl, arylsulfonyl, acylamino, aminoacyl, aminothioacyl, amidino, mercapto, amido, thioureido, thiocyanato, sulfonamido, guanidine, ureido, cyano, nitro, acyl, thioacyl, acyloxy, carbamido, carbamyl (—C(O)NH₂), carboxyl (—COOH), and carboxylic ester. On the other hand, possible substituents on alkyl, alkenyl, alkynyl, or alkylene include all of the above-recited substituents except C₁-C₁₀ alkyl. Cycloalkyl, cycloalkenyl, heterocycloalkyl, heterocycloalkenyl, aryl, and heteroaryl can also be fused with each other.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of 0, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(═O)R_{B}” group in which RA and R_{B} are each independently selected from hydrogen, C₁₋₆ alkyl, C₂₋₆ alkenyl, C₂₋₆ alkynyl; C₃₋₇ carbocyclyl, C₆₋₁₀ aryl, 5-10 membered heteroaryl, and 5-10 membered heterocyclyl, as defined herein.

An “0-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂-6 alkynyl, C₃₋₇ carbocyclyl, C₆₋₁₀ aryl, 5-10 membered heteroaryl, and 5-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substitutents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl (C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

In some embodiments, substituted group(s) is (are) substituted with one or more substituent(s) individually and independently selected from C₁-C₄ alkyl, amino, hydroxy, and halogen.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

As used herein, “alkylene” means a branched, or straight chain fully saturated di-radical chemical group containing only carbon and hydrogen that is attached to the rest of the molecule via two points of attachment (i.e., an alkanediyl). The alkylene group may have 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene.

As used herein, “alkenylene” means a straight or branched chain di-radical chemical group containing only carbon and hydrogen and containing at least one carbon-carbon double bond that is attached to the rest of the molecule via two points of attachment. The alkenylene group may have 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene” or similar designations. By way of example only, “C₂₋₄ alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, but-1-enylene, but-2-enylene, but-1,3-dienylene, buten-1,1-diyl, but-1,3-dien-1, 1-diyl, but-2-en-1,1-diyl, but-3-en-1,1-diyl, 1-methyl-prop-2-en-1,1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2,2-dimethyl-ethen-1,1-diyl.

The term “agent” or “test agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved characteristics (such as higher binding affinity for a target molecule) is an approach that is well known in pharmaceutical chemistry.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rats and mice but also includes many other species.

The term “microbial infection” refers to the invasion of the host organism, whether the organism is a vertebrate, invertebrate, fish, plant, bird, or mammal, by pathogenic microbes. This includes the excessive growth of microbes that are normally present in or on the body of a mammal or other organism. More generally, a microbial infection can be any situation in which the presence of a microbial population(s) is damaging to a host mammal. Thus, a mammal is “suffering” from a microbial infection when excessive numbers of a microbial population are present in or on a mammal's body, or when the effects of the presence of a microbial population(s) is damaging the cells or other tissue of a mammal. Specifically, this description applies to a bacterial infection. Note that the compounds of preferred embodiments are also useful in treating microbial growth or contamination of cell cultures or other media, or inanimate surfaces or objects, and nothing herein should limit the preferred embodiments only to treatment of higher organisms, except when explicitly so specified in the claims.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

### Administration and Pharmaceutical Compositions

The compounds are administered at a therapeutically effective dosage. While human dosage levels have yet to be optimized for the compounds described herein, generally, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21 st Ed., Lippincott Williams & Wilkins (2005), incorporated by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

In addition to the selected compound useful as described above, come embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

### Methods of Treatment

Some embodiments of the present invention include methods of treating microbial infections with the compounds and compositions comprising the compounds described herein. Some methods include administering a compound, composition, pharmaceutical composition described herein to a subject in need thereof. In some embodiments, a subject can be an animal, e.g., a mammal (including a human). In some embodiments, the bacterial infection comprises a bacteria described herein. As will be appreciated from the foregoing, methods of treating a bacterial infection include methods for preventing bacterial infection in a subject at risk thereof.

In some embodiments, the subject is a human.

Further embodiments include administering a combination of compounds to a subject in need thereof. A combination can include a compound, composition, pharmaceutical composition described herein with an additional medicament.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered i.v.

Examples of additional medicaments include an antibacterial agent, antifungal agent, an antiviral agent, an anti-inflammatory agent and an anti-allergic agent.

The compounds and compositions comprising the compounds described herein can be used to treat microbial infections. Microbial infections that can be treated with the compounds, compositions and methods described herein can comprise a wide spectrum of bacteria. Example organisms include gram-positive bacteria, gram-negative bacteria, aerobic and anaerobic bacteria.

The pharmaceutical composition of the present invention may also comprise a pharmaceutically acceptable excipient. Therefore, the present invention is also directed to a pharmaceutical composition as disclosed above, wherein the pharmaceutical composition additionally comprises a pharmaceutically acceptable excipient.

In general all excipients known by a person skilled in the art are suitable within the present invention. Examples of such excipients are calcium carbonate, kaolin, sodium hydrogen carbonate, lactose, D-mannitol, starches, crystalline cellulose, talc, granulated sugar, porous substances, etc.

The compounds of formula (I) of the invention may be used as bulk itself but usually be formulated into pharmaceutical preparations together with a suitable amount of “carrier for pharmaceutical preparation” according to ordinary methods.

Thus, compositions and methods according to the invention may also contain additionally diluents, fillers, salts, buffers, stabilizers, solubilizers, and other materials well known in the art.

Further on, “carriers for pharmaceutical preparation” comprises, for example, excipients as defined above, binders, e.g., dextrin, gums, α-starch, gelatin, hydroxypropyl cellulose, hydroxypropylmethyl cellulose, pullulan, etc., thickening agents, e.g., natural gums, cellulose derivatives, acrylic acid derivatives, etc., disintegrators, e.g., carboxy-methyl cellulose, croscarmellose sodium, crospovidone, low-substitution hydroxypropyl cellulose, partial α-starch, etc., solvents, e.g., water for injections, alcohol, propylene glycol, macrogol, sesame oil, corn oil, etc., dispersants, e.g., Tween 80, HC060, polyethylene glycol, carboxymethyl cellulose, sodium alginate, etc., solubilizers, e.g., poly-ethylene glycol, propylene glycol, D-mannitol, benzyl benzoate, ethanol, trisami-nomethane, triethanolamine, sodium carbonate, sodium citrate, etc., suspending agents, e.g., stearyl triethanolamine, sodium lauryl sulfate, benzalkonium chloride, polyvinyl alcohol, polyvinylpyrrolidone, hydroxyethyl cellulose, etc., pain-reducing agents, e.g., benzyl alcohol, etc., isotonizing agents, e.g., sodium chloride, glycerin, etc., buffers, e.g., phosphates, acetates, carbonates, citrates, etc., lubricants, e.g., magnesium stearate, calcium stearate, talc, starch, sodium benzoate, etc., colorants, e.g., tar pigments, caramel, iron sesquioxide, titanium oxide, riboflavins, etc., tasting agent, e.g., sweeteners, flavors, etc., stabilizers, e.g., sodium sulfite, ascorbic acid, etc., preservatives, e.g., parabens, sorbic acid, etc., and the like.

Pharmaceutical compositions according to the present invention may also comprise other active factors and/or agents which enhance the inhibition of enzymes which reduce, destroy the activity of compounds of Formula (I) or factors and/or agents which enhance inhibition of beta-lactamases and/or DD-peptidases.

The respective pharmaceutical compositions are effective against bacteria which do not produce enzymes which reduce, destroy the activity of compounds of Formula (I), but also especially effective against bacteria which produce significant amounts of enzymes which reduce, destroy the activity of compounds of Formula (I). Thus, pharmaceutical compositions according to the present invention are generally useful for controlling microbial infections levels in vivo and for treating diseases or reducing the advancement or severity of effects, which are mediated by bacteria.

Suitable subjects for the administration of the formulation of the present invention include mammals, primates, man, and other animals. Typically the animal subject is a mammal, generally a domesticated farm mammal, e.g. horse, pig, cow, sheep, goat etc., or a companion animal, e.g. cat, dog etc. In vitro antibacterial activity is predictive of in vivo activity when the compositions are administered to a mammal infected with a susceptible bacterial organism.

### Route of Administration

Preferred methods of administration of the pharmaceutical compositions described above include oral and parenteral, e.g., i.v. infusion, i.v. bolus and i.m. injection formulated so that a unit dosage comprises a therapeutically effective amount of each active component or some submultiples thereof. The compounds may be employed in powder or crystalline form, in liquid solution, or in suspension. These compounds may be formulated by any method well known in the art and may be prepared for administration by any route, including, without limitation, parenteral, oral, sublingual, by inhalation spray, transdermal, topical, intranasal, intra-tracheal, intrarectal via ophthalmic solution or ointment, rectally, nasally, buccally, vaginally or via implanted reservoir. The term parenteral as used herein includes subcutaneous, intracutaneous, intravenous, intramuscular, intra-articular, intrasynovial, intrasternal, intrathecal, intralesional and intracranial injection or infusion techniques.

In case the compound of formula (I) is an acid, the pharmaceutical composition according to the present invention is preferably administered parenteral, in particular intravenous. In case the compound of formula (I) is an ester, the pharmaceutical composition according to the present invention is preferably administered orally.

Pharmaceutical compositions for injection, a preferred route of delivery according to the present invention, may be prepared in unit dosage form in ampules, or in multidose containers. The composition will generally be sterile and pyrogen-free, when intended for delivery by injection into the subject. The injectable compositions may take such forms as suspensions, solutions, or emulsions in oily or aqueous vehicles, and may contain various formulating agents. Alternatively, the active ingredient may be in powder (lyophilized or non-lyophilized) form for reconstitution at the time of delivery with a suitable vehicle, such as sterile water.

Carriers suitable for an injectable pharmaceutical composition according to the present invention are typically comprised sterile water, saline or another injectable liquid, e.g., peanut oil for intramuscular injections. Also, various buffering agents, preservatives and the like can be included. The pharmaceutical composition according to the present invention may also be administered parenterally in a sterile medium. Depending on the vehicle and concentration used, the drug can either be suspended or dissolved in the vehicle. Advantageously, adjuvants such as local anaesthetic, preservative and buffering agents can be dissolved in the vehicle. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. It is also preferred to include isotonic agents, for example, sugars, buffers or sodium chloride. Prolonged absorption of the injectable compositions can be brought about by the use in the compositions of agents delaying absorption, for example, aluminium monostearate and gelatine. Intra-venous infusion is another possible route of admini-stration for the compounds used according to the present invention. Orally administrable pharmaceutical compositions according to the present invention may be in the form of tablets, capsules, powders, granules, lozenges, liquid or gel preparations, such as oral, topical, or sterile parenteral solutions or suspensions. The oral compositions may utilize carriers such as conventional formulating agents, and may include sustained release properties as well as rapid delivery forms. Such compositions and preparations should contain at least 0.1% of active compounds. The percentage of the compositions and preparations may, of course, be varied and may conveniently be between about 2 to about 60% of the weight of a given unit dosage form. The amount of active compound in such therapeutically useful compositions is such that an effective dosage level will be obtained.

Tablets and capsules for oral administration may be in unit dose presentation form, and may also contain conventional excipients such as binding agents, for example syrup, acacia, gelatine, sorbitol, tragacanth, or polyvinylpyrrolidone; fillers for example lactose, sugar, maize-starch, calcium phosphate, sorbitol or glycine; tabletting lubricant, for example magnesium stearate, talc, polyethylene glycol or silica; disintegrates for example potato starch, or acceptable wetting agents such as sodium lauryl sulphate. The tablets may be coated according to methods well known to a person skilled in the art. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatine hydrogenated edible fats; emulsifying agents, for example lecithin, sorbitan monooleate, or acacia; non-aqueous vehi-cles which may include edible oils, for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

Pharmaceutical compositions according to the present invention may also be prepared in suitable forms for absorption through the mucous membranes of the nose and throat or bronchial tissues and may conveniently take the form of powder or liquid sprays or inhalants, lozenges, throat paints, etc. For medication of the eyes or ears, the preparations may be presented as individual capsules, in liquid or semi-solid form, or may be used as drops, etc.

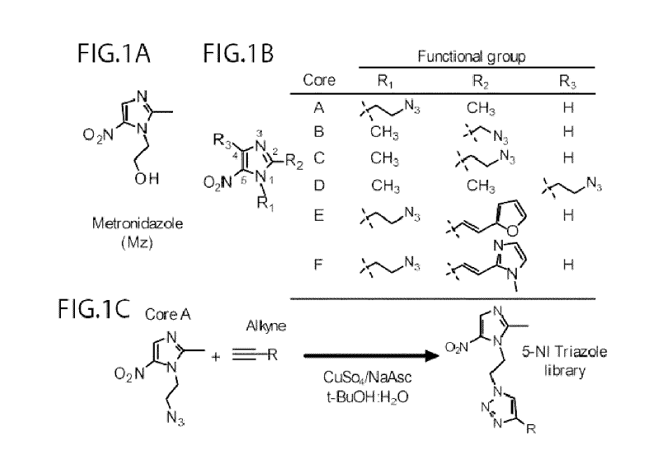

We first synthesized six azido derivatives of 5-NI, in which one of the three available positions in the imidazole ring was functionalized with azidoalkyl groups (FIG. 1B). FIG. 1 shows the synthesis of a comprehensive new 5-NI library. FIG. 1A shows the structure of metronidazole (Mz). FIG. 1B shows six different 5-NI cores (A-F) which were synthesized with a “clickable” azide (N₃) functional group. FIG. 1 C shows the scheme of click chemistry-facilitated synthesis of 5-NI triazole library. The distance between the azido group and the imidazole core was minimized to limit structural restrictions on the products of the subsequent cycloaddition, although direct linkage of the azido group to the imidazole core proved impossible, presumably because it compromised the stability of the heterocycle. We also generated 1-azidoalkyl imidazole compounds with conjugated furan- or imidazole-based side chain in the 2-position (cores E and F, FIG. 1B), as our previous studies had suggested that these modifications can yield potent antimicrobials (6).

We next assembled a library of 63 structurally diverse alkynes as reaction partners for the azido cores (Table S1). The alkynes, which were obtained commercially or synthesized de novo, were selected to achieve maximal structural diversity with little consideration of conventional pharmacological criteria such as hydrophobicity, electrochemical properties, or previously successful side chains. We then performed all possible copper(I)-catalyzed cycloadditions of the six azido-imidazole cores and 63 alkynes to synthesize 378 new 5-NIs (FIG. 1C, Table S1). Initial studies showed similar antimicrobial activities in crude reaction mixtures compared to purified compounds, indicating that the reaction mixtures could be rapidly screened for activity without the time-consuming isolation of the products.

The following examples will further describe the present invention, and are used for the purposes of illustration only, and should not be considered as limiting.

### EXAMPLES

### Example 1

### Broad Antimicrobial Activities of Diverse 5-NI Compounds

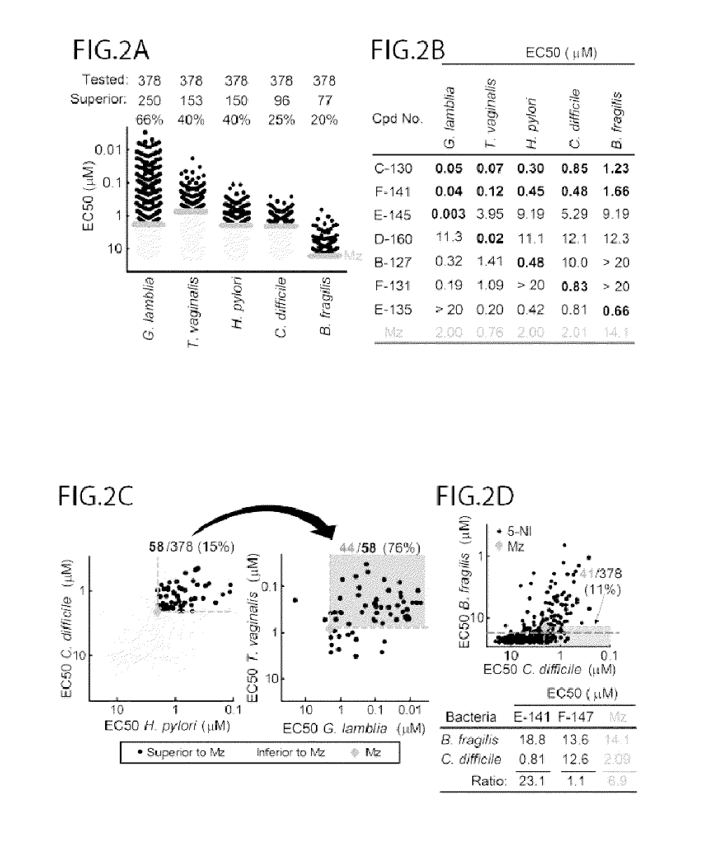

As microbial targets and test system for the new 5-NI library, we used a range of clinically important protozoa and bacteria that are commonly treated with Mz. The intestinal parasite, G. lamblia, is a major worldwide protozoan cause of diarrheal disease (21) and T. vaginalis is a common protozoal cause of sexually transmitted disease of the genitourinay tract (22), while H. pylori and C. difficile are major bacterial causes of infectious disease in stomach and colon, respectively (23, 24). Using quantitative growth and survival assays, we found that 66% of the 378 tested compounds had superior activity relative to Mz against G. lamblia and 40% against T. vaginalis, with up to 50- to 500-fold lower EC50 relative to Mz (FIG. 2A, Tables S2 and S3). Marked activity improvements were also observed for the bacterial pathogens, since 40% of the compounds were superior to Mz against H. pylori and 25% against C. difficile (FIG. 2A, Table S4).

FIG. 2 shows the expanded antimicrobial activity range of new 5-NI compounds. FIG. 2A shows the activities of 378 compounds that were tested against the indicated protozoa and bacteria in 24-48 h growth assays using ATP levels or OD600 as read-outs. Each data point or number represents the mean EC50 for one compound, with Mz shown in solid lines in FIG. 2A for comparison. FIG. 2B lists examples of compounds with enhanced broad-spectrum or pathogen-selective activity (key values in bold). FIG. 2C displays relationships between activities of individual compounds against the four target pathogens. Compounds that exceeded the activity of Mz (diamond point) for both bacteria (black circles in light grey-shaded region, left panel) were examined for their activities against the two protozoa (right panel). The region containing compounds with superior activity against all four pathogens is shaded in darker grey. FIG. 2D shows the relationship between compound activities against two colonic bacteria. The orange-shaded region highlights compounds more active than Mz against the pathogen, C. difficile, but less active than Mz against the commensal, B. fragilis. Assay sensitivity is depicted by the dashed line in FIG. 2D. The table lists examples of compounds that showed greater or lesser selectivity than Mz against C. difficile compared to B. fragilis.

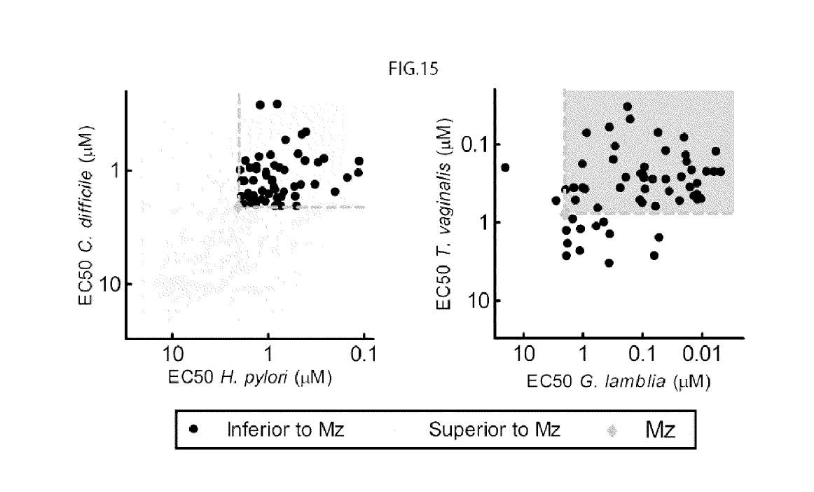

FIG. 15 shows the expanded antimicrobial activity range of new 5-NI compounds. The activities of 378 new 5-NI compounds were tested against G. lamblia (strain 713), T. vaginalis (strain G3), H. pylori (strain SS1) and C. difficile (ATCC 9689) in 24-48 h growth assays, using ATP content or OD600 as read-outs. Each data point represents the mean value for one compound, with Mz shown in diamond dot for comparison. The graphs depict relationships between activities of individual compounds against the four target pathogens. The left panel shows correlations for antibacterial activities. The region with compounds that exceeded the activity of Mz for both bacteria is shaded light red. These superior compounds (black circles) were then analyzed for their activities against the two protozoa (right panel). The region containing compounds with superior activity against all four pathogens is shaded in dark red.

Some compounds displayed highly selective improvement in activity against one of the target microbes, while others exhibited greater activity against more than one microbe (FIG. 2B, Tables S2-S4). Analysis of the relationships between different antimicrobial activities revealed modest but significant positive correlations between the protozoa (r=0.38, p<0.001) and bacteria (i=0.53, p<0.001) (FIG. 2C and FIG. 15). Moreover, superior antibacterial activity correlated with improved antiprotozoal activity, and conversely, superior antiprotozoal activity correlated with better antibacterial activity (FIG. 2C and FIG. 15). For example, 44 of 58 (76%) of the compounds that were more active than Mz against both bacterial pathogens were also more active against both protozoa. Together, 12% (n=44) of the 378 tested compounds were superior to Mz against all four pathogens (FIG. 2C). This percentage of broadly active compounds significantly exceeded (by >4-fold) the predicted 2.6% of compounds if improvements in activity against each individual microbe were entirely independent from improvement against the other microbes, as calculated by multiplying the individual percentages of superior compounds for each of the four target pathogens (i.e., 66%, 40%, 40% and 25%; FIG. 2A).

Given the broad-spectrum activity of several compounds, we questioned whether the nitro compounds were also active against intestinal commensals, which might impact their clinical utility. None of the 378 nitro compounds exhibited activity against the commensal Escherichia coli (strain K12) up to a maximum concentration of 20 μM, indicating that the new 5-NI drugs, like Mz, do not have non-specific toxicity in microbes naturally resistant to this drug class. B. fragilis is an important commensal that normally resides in the intestinal lumen, but can cause peritoneal infections when translocated due to gut perforations. A substantial fraction (20%) of compounds had superior activity against B. fragilis compared to Mz (FIG. 2A,B and Table S4). Importantly, comparison of activities against B. fragilis and C. difficile, both of which colonize the colon, revealed a range of ratios (FIG. 2b), indicating that nitro drugs exist with improved selectivity against the pathogen C. difficile compared to the commensal B. fragilis.

Collectively, these results indicate that many new 5-NIs have a marked, and for some highly selective, increase in activity against each of the individual microbes. Furthermore, a significantly greater than expected number of compounds were superior to Mz against multiple different microbes, making these compounds strong candidates for broad-spectrum antibiotics. Importantly, the vast majority of new compounds (362/378, >95%) had no cytotoxicity in human HeLa cells and many showed therapeutic indices equal or superior to Mz (Table S5).

### New 5-NI Compounds can Overcome Different Forms of Mz Resistance

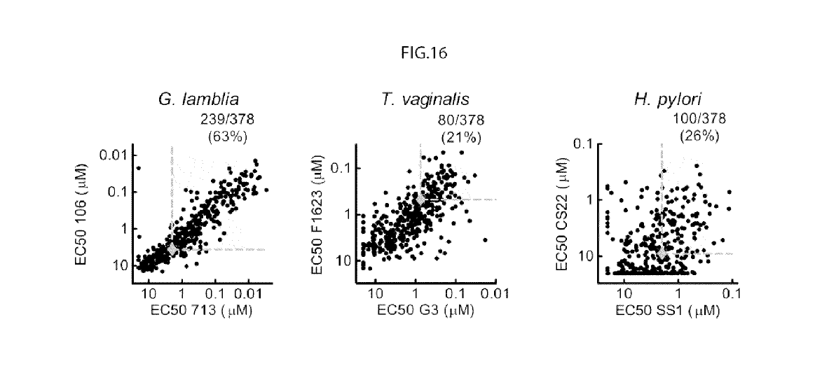

FIG. 16. Relationship of compound activities between clinical isolates. The 5-NI library was tested for activity against two different clinical Mz-sensitive (MzS) isolates of G. lamblia (strains 713 and 106), T. vaginalis (strains G3 and F1623), and H. pylori (strains SS1 and CS22). Compounds with greater activity than Mz (diamond point) against both of the respective Mz-sensitive isolates are highlighted by light grey shading (and their numbers and percentages of all tested compounds are given above the region).

Natural variability in antimicrobial susceptibility is an important consideration in new drug development. To address this issue, we tested additional isolates of three of the target pathogens and related the data to the initial screens. Activities showed generally good correlations (with r values of 0.896, 0.695, and 0.388 for Giardia, Trichomonas, and Helicobacter, respectively; p<0.01 for all three; FIG. 16, Tables S2-S4), but differences were apparent between the pathogens. For Giardia, 63% of the compounds exhibited superior activity over Mz against both clinical isolates, while this percentage was lower for T. vaginalis (21%) and H. pylori (26%), underlining the importance of testing a range of isolates to avoid pursuit of compounds with limited, isolate-specific activity. Nonetheless, these data demonstrate that the 5-NI library had multiple compounds with superior activities against different clinical isolates of the target pathogens.

Beyond natural variability in antimicrobial susceptibility, resistance to Mz can develop during antimicrobial therapy, and Mz resistance can be readily induced in bacteria and protozoa in the laboratory (10). Importantly, currently approved 5-NI drugs exhibit cross-resistance to Mz and show similar clinical failure rates (10). To determine whether the new 5-NI compounds can overcome acquired Mz resistance, we employed two independently derived syngeneic Mz-resistant (MzR) lines of G. lamblia and two clinical MzR isolates of T. vaginalis.

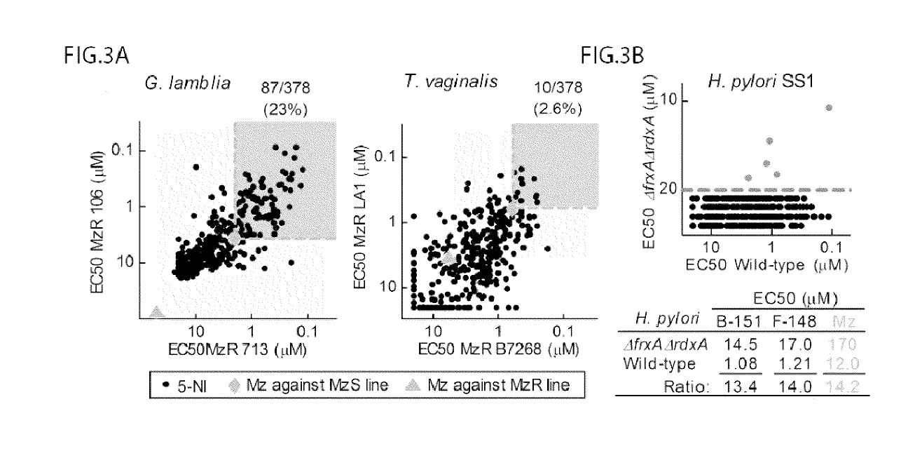

FIG. 3 shows new 5-NI compounds overcome Mz resistance. Compounds were tested against MzR strains of G. lamblia and T. vaginalis, with those more active than Mz (purple) highlighted by light grey shading boxes (FIG. 3A). Of these, compounds more active against both MzR lines than Mz against the respective MzS lines (dashed line in FIG. 3A) are further highlighted by dark grey shading boxes (FIG. 3A). Activities against wild-type and ΔfrxA ΔrdxA strains of H. pylori are shown in FIG. 3B. In FIG. 3B, five compounds (black dots above the dashed line) had measurable activity against the mutant, all others were below the assay sensitivity (dashed line). Examples of active compounds are listed in the table (FIG. 3B).

Remarkably, all of the 378 tested compounds exhibited activity against the two MzR Giardia lines that was superior (i.e., lower EC50) to Mz (FIG. 3A, light grey shading; Table S2). Moreover, 23% of the compounds (n=87) were also more active against both two MzR lines than Mz was against the corresponding parental Mz-sensitive (MzS) lines (FIG. 3A, darker grey shading). Thus, these 87 compounds effectively overcame acquired Mz resistance in Giardia, as they could kill MzR lines at least as effectively as Mz could kill MzS lines.

For T. vaginalis, 176 of 377 tested compounds (47%) were superior to Mz in two unrelated MzR isolates (FIG. 3A, light grey shading; Table S3). Of these, 10 compounds (2.6% of all compounds) exhibited activity against both MzR lines that exceeded Mz activity against the MzS lines (FIG. 3A, dark grey shading). Thus, the range of activities, if not the frequency of compounds with superior activity, against T. vaginalis was similar to G. lamblia, supporting the conclusion that our comprehensive 5-NI drug library contains compounds with diverse activities against a wide range of microbes and forms of drug resistance.

As a further test of the ability to overcome nitro drug resistance, we employed a double mutant of H. pylori for two reductases, FrxA and RdxA, that activate nitro drugs and are involved in clinical drug resistance (25). Screening of the library revealed that 98.6% of compounds lost activity against the double mutant (FIG. 3B, Table S4), which demonstrates that reduction by one or both of these reductases is critical for compound activity and further underlines the high molecular specificity of the new compounds. Interestingly, we also found five compounds that remained active in the 10-20 μM range in the double mutant, suggesting that alternative reductive pathways may exist in H. pylori that can activate these nitro compounds.

### Distinct and Predictive Bioactivity Landscapes of 5-NI Compounds

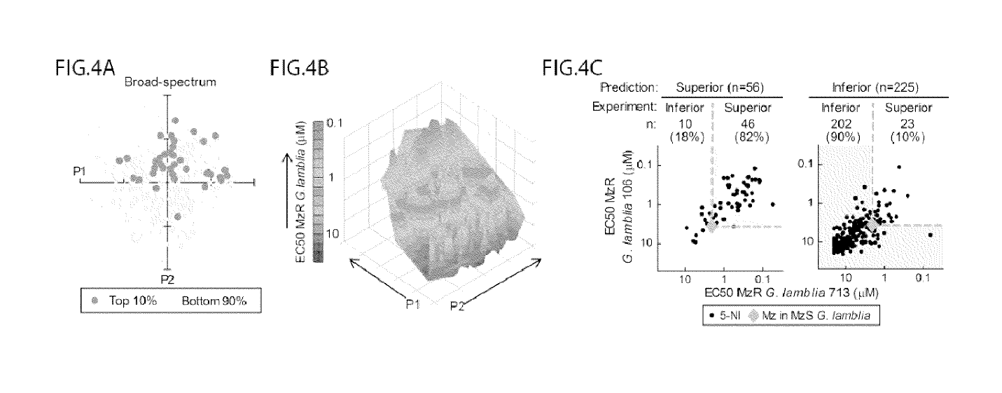

FIG. 4 shows some examples of bioactivity landscape of 5-NI compounds. A structural space was generated by principal component analysis using activity data of the 378 compounds in the 5-NI library against the four target microbes. The individual compounds were plotted in the resulting space with the top 10% most potent broad-spectrum compounds shown as solid black dots (FIG. 4A). In FIG. 4B, a structural space was constructed from the activity data against MzR G. lamblia. Activities of all compounds were plotted in the space (Z-axis), and a contour graph was generated using the indicated color scale. In FIG. 4C, a service vector machine model was constructed from the activity data of the 378 compounds against MzR Giardia (training set), and applied prospectively to a new set of 281 independently synthesized 5-NI compounds (test set). Correctly predicted compounds, as defined by coincidence of model prediction and assay-determined activity against the two MzR Giardia lines, are highlighted by background coloring (light grey regions in the left panel and right panel in FIG. 4C). Each data point represents one compound. The diamond dots show Mz activity against the MzS parental lines for comparison.

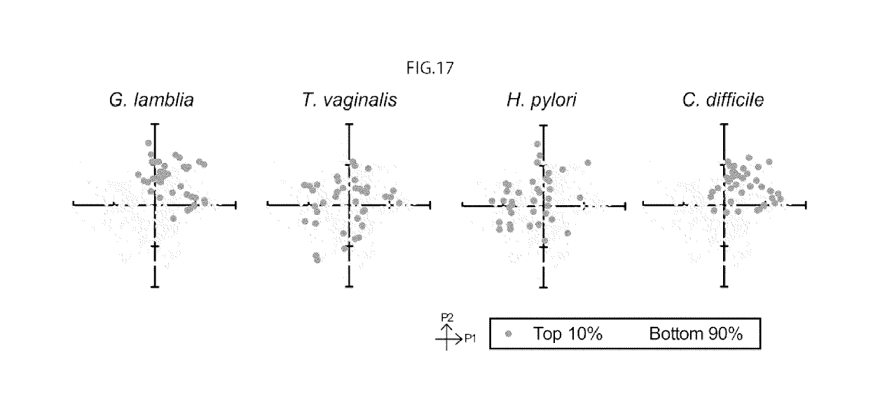

FIG. 17 shows a distribution of active 5-NI compounds in chemical space. A structural space was generated by principal component analysis using activity data of all 378 compounds in the 5-NI library against the four target microbes, and the individual compounds were plotted in the resulting space. The top 10% most potent compounds are shown in solid black dots for activity against Mz-sensitive (MzS) isolates of each of the indicated pathogens. All other compounds are shown in light gray.

To begin to understand the relationship between compound structures and antimicrobial activities, we employed principal component analysis to visualize the 5-NI compounds in a structural space and then highlighted the most active compounds within that space. The top 10% compounds with broad-spectrum activity against all four target pathogens were located in different regions within the space (FIG. 4A). By comparison, localization of the most active compounds against each of the individual pathogens revealed distinct patterns (“activity fingerprints”) for the four pathogens (FIG. 17), which overlapped only partly with the pattern of broad-spectrum compounds. These data suggest that the structural requirements for activity improvement heavily depend on the particular microbial targets, but also show that broad-spectrum activity can be achieved in multiple structural domains.

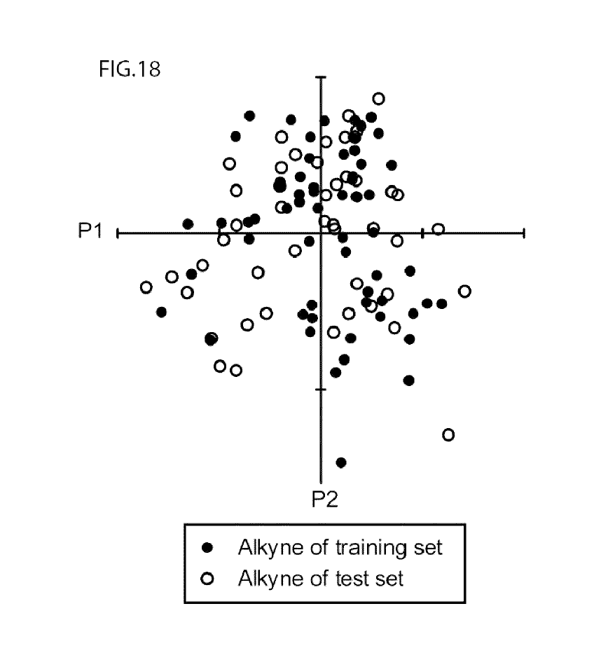

FIG. 18 shows the structural analysis of alkyne sets used in library generation. Alkynes used for synthesizing the training triazole compounds (closed circles) or test triazole compounds (open circles) were analyzed by principal component analysis for their distribution in a common structural space.

Further evaluation of the structure-activity relationships by principal component analysis for one of the pathogens, MzR G. lamblia, revealed a non-random distribution of bioactivity in the chemical space occupied by the compounds, with distinct activity peaks and valleys (FIG. 4B). Based on this observation, we applied machine learning tools to construct a mathematical model for bioactivity prediction. While decision tree analysis yielded excellent (84%) cross-validation in the set of 378 training compounds, even better predictability (87%) was achieved with a service vector machine approach. To test the predictive power of the model, we synthesized a new set of 281 test compounds by click chemistry using the same six 5-NI cores A-F (FIG. 1B) but 47 new alkynes (Table S6). These alkynes were structurally different from those employed in the initial library, but occupied an overlapping chemical space (FIG. 18). The model was used to divide the new test compounds into two groups with predicted superior or inferior activity in MzR cells relative to Mz in the parental MzS Giardia cells. Some 82% of predicted superior compounds exhibited superior activity in antimicrobial testing, while 90% of predicted inferior compounds had inferior activity in the tests (FIG. 4C, Table S7). Together, bioactivity of 86% (248 of 281) of the test compounds against MzR G. lamblia was correctly predicted, indicating the excellent utility of the predictive model. The findings experimentally validate prior contentions that structural analysis by chemical descriptors can be exploited to define chemical spaces with promising structure-activity relationships (26-28). As for the training compounds, the vast majority of test compounds (260/279, 93%) had no measurable cytotoxicity in human HeLa cells (Table S8).

### Structural Determinants of Superior 5-NI Activity

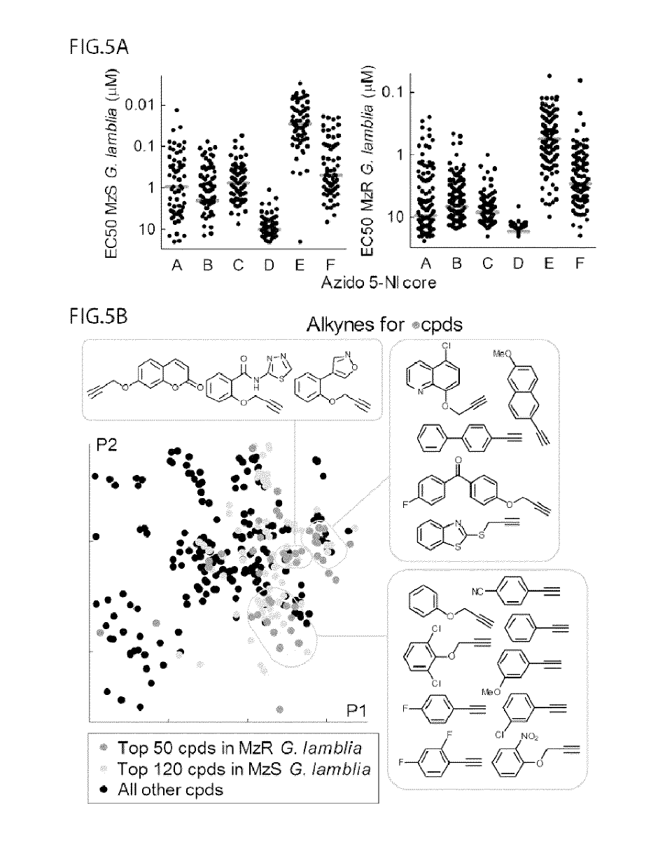

FIG. 5 shows structure-activity relationships of 5-NI building blocks. The entire library of 659 5-NI compounds (composed of the 378 training and 281 test compounds) was examined for structure-activity relationships of the two building blocks, azido-5-NI and alkyne, used for the click chemistry-facilitated synthesis. FIG. 5A shows the influence of the azido-5-NI cores (A-F, see FIG. 1B) on activity against Mz-sensitive (MzS, left) and Mz-resistant (MzR, right) Giardia, with average activities shown as solid lines in the figure. FIG. 5B depicts data of all compounds generated from cores A-C (to minimize core bias in the alkyne evaluation) in a structural space derived by principal component analysis. The top most active compounds against MzS and MzR Giardia are highlighted in light brown and red, respectively. Several regions with clustering of the most active compounds are boxed, and the corresponding alkynes used for compound synthesis are depicted.

To define the structural determinants of superior antimicrobial activity of the new 5-NI triazole compounds, we first examined the importance of the azido-imidazole core. Compounds based on cores A-C had similar average activities against MzS and MzR Giardia (FIG. 5A). Core D compounds had low or no activity against either, while the E series compounds had, on average, significantly higher activities than those of the other cores against both MzS and MzR Giardia (FIG. 5A). To explore the contribution of the alkyne partners to triazole bioactivity, we focused on the antigiardial activity of A-C series compounds to minimize core bias. Principal component analysis revealed a non-random, patchy distribution of the superior triazoles, occupying several distinct areas in the chemical space of the entire group of core A-C compounds (FIG. 5B). The corresponding alkynes in the active regions showed marked structural similarities within each of these regions, but little between different regions. These results indicate that several structurally unrelated groups of compounds can yield superior antigiardials.

### In Vivo Efficacy of New 5-NIs in Animal Infection Models

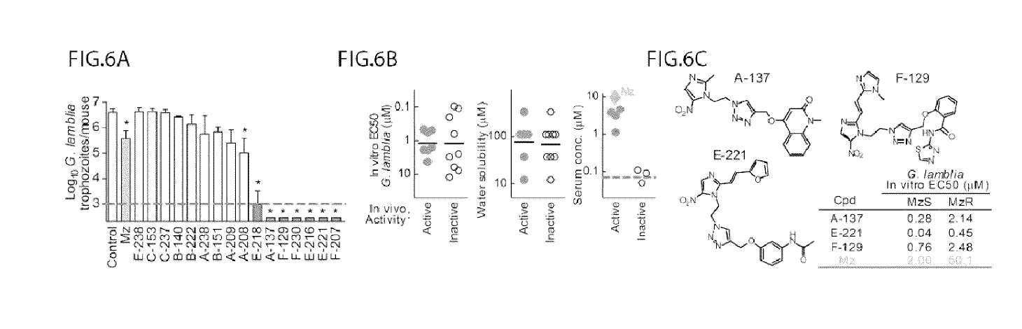

FIG. 6 shows in vivo efficacy of 5-NI compounds against giardiasis. Adult C57BL/6 mice were orally infected with G. lamblia, given five 10 mg/kg oral doses of the indicated compounds over three days at 12 h intervals, and trophozoite numbers in the small intestine were determined (FIG. 6A). The dashed line in FIG. 6 represents the assay sensitivity. Data are mean+SEM (n≧5); *p<0.05 vs vehicle-treated controls. Compounds significantly more efficacious than Mz (diamond) are highlighted in solid black dots. The relationships of in vivo bioactivity, in vitro activity, aqueous solubility, and measured serum drug concentrations are shown in FIG. 6B (dashed line in FIG. 6A represents the assay sensitivity; Mz is shown as a diamond for comparison). Examples for in vivo active compounds are given in FIG. 6C, along with their in vitro activities against MzS and MzR Giardia.

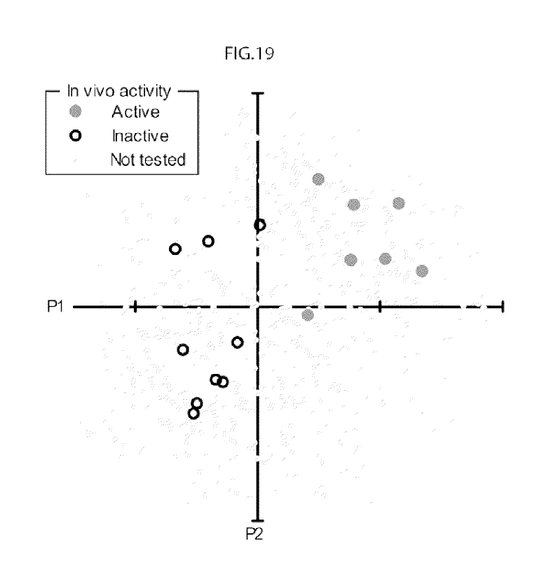

FIG. 19 shows the structural analysis of in vivo active 5-NI compounds. A structural space was generated by principal component analysis for all 659 compounds in the total 5-NI library. Compounds that were efficacious in the mouse giardiasis model are shown as solid black dots. Compounds not active in vivo are shown as black open circles, while the small gray dots represent all other 5-NI compounds in the library not test in vivo.

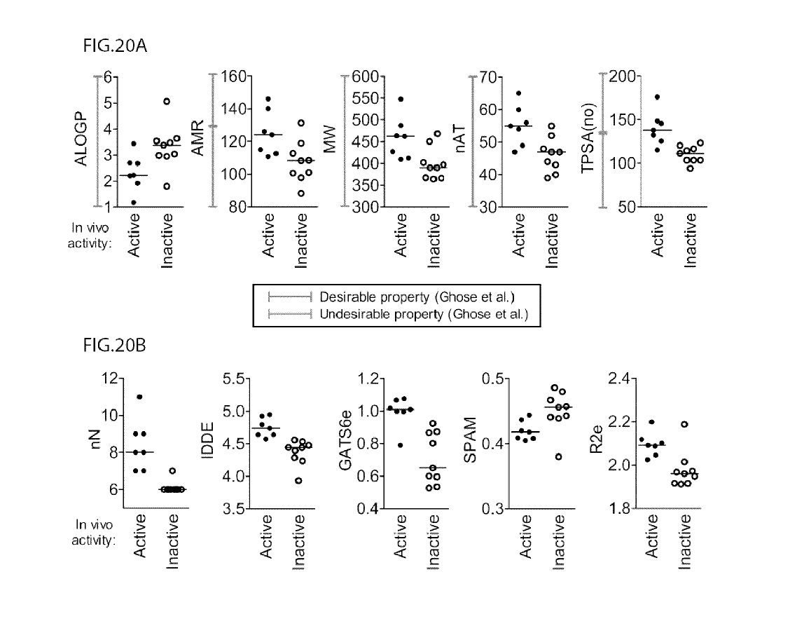

FIG. 20 shows the chemical descriptors for prediction of in vivo activity. A. Comparison of five chemical descriptors by Lipinski's profiling tool. Each data point shows the value of the indicated chemical descriptor for compounds tested in vivo. Active compounds (closed circles) cleared Giardia infection in mice, while inactive compounds (open circles) did not (see FIG. 6). Means are shown as horizontal lines for each group. Vertical lines on the left of each panel/figure indicate the preferred range for drug likeness as characterized by Ghose et al. (29); solid line, preferable; dashed line, not preferable. B. Comparison of five alternative chemical descriptors for prediction of in vivo activity, as selected by a machine learning algorithm trained by data from the in vivo studies. These descriptors were used for the principal component analysis shown in Fig. S5. Explanations: nN, Number of nitrogen atoms; IDDE, Mean information content on the distance degree equality; GATS6e, Geary autocorrelation of lag 6 weighted by Sanderson electronegativity; SPAM, Average span R geometrical descriptors; R2e, R autocorrelation of lag 2 weighted by Sanderson electronegativity.

Because many 5-NI compounds exhibited promising antimicrobial activities in vitro, we next examined their in vivo efficacy in a murine model of giardiasis. Out of 16 structurally representative compounds, 8 (50%) displayed significant activity against giardiasis, and 7 (45%) were more efficacious at clearing Giardia than Mz at a standard dose of 10 mg/kg (FIG. 6A, red). Acute toxicity was not observed over the 3-day treatment course. In vivo efficacy could not be explained by greater in vitro antigiardial activity or altered aqueous solubility (FIG. 6B). However, active compounds had higher serum levels than inactive compounds by 2 h after oral administration (FIG. 6B), suggesting that systemic bioavailability was important for antigiardial efficacy. Examination of the structure-activity relationships revealed five chemical attributes, different from those in the Lipinski rules commonly used to predict bioavailability of new compounds (29, 30), that could effectively differentiate in vivo active and inactive compounds in the chemical space of all library compounds (FIGS. 19 and 20) (31). Examples of active compounds show that promising activities against giardiasis can be found among very different chemical structures (FIG. 6C). Initial evaluation of the toxicity profile of several of these compounds by micronucleus assay with mammalian CHO cells using current OECD guidelines, and by liver enzyme panels and histological and hematological evaluation of adult mice dosed five times with 100 mg/kg (˜10× effective dose) over three days revealed no toxicities beyond the safe drug Mz used for comparison.

Our results indicated that next-generation 5-NI drug candidates can be synthesized and rapidly tested in vitro and in vivo by combining the case of modular click chemistry with cell culture assays, predictive machine learning tools, and animal models. Thus, it is readily feasible to advance from initial synthesis of hundreds compounds to focused animal testing in mere weeks, thereby significantly shortening the initial development cycle in drug design (20). This acceleration could promote a timely and cost-effective drug development response to the emergence of antimicrobial resistance that threatens public health. Adequate preclinical safety testing are studied before commencing human trials, but even this step can be significantly shortened and made more cost-effective by rapid production of kilogram quantities of new drug candidates by click chemistry or other chemistries suitable for combinatorial synthesis and ready side chain preservation.

The observed increases in antimicrobial activity were striking (>100-fold) for some of the new compounds, although the underlying reasons remain to be established. Despite the widespread use of Mz and other 5-NI drugs, their mechanisms of antimicrobial killing remain incompletely understood. Reduction of the prodrugs is required for activity, so higher affinity or faster reaction kinetics of new compounds with the relevant reductases may be important. The stability or molecular targeting efficiency of activated nitro drug radicals may be more favorable for the new compounds (32). Alternatively, the spectrum of nitro drug adduction targets may be different for the most potent compounds (6, 33). In any case, the observation that many compounds showed improved broad-spectrum activity would suggest that the underlying mechanisms are likely to entail broadly relevant drug properties, such as improved membrane permeability, that are not limited to particular molecular targets or microbial features.

A key observation of our studies is that many new compounds could effectively overcome nitro drug resistance in different microbes. Mz resistance is functionally heterogeneous with several causative mechanisms for each of the target microbes and between different microbes (4, 34). For example, resistance in Giardia involves downregulation of nitro drug-activating systems, including different reductases and redox proteins and metabolites (12, 35), while Trichomonas also produces resistance proteins that directly detoxify nitro drugs (14) and Bacteroides has transporters that can remove nitro drugs from the cell (36). Despite this mechanistic diversity, for each of the resistant microbes, we were able to identify nitro compounds that could combat resistance, and some compounds were broadly effective against different resistant microbes. Several basic explanations, alone or in combination, may account for this finding. New nitro compounds may be activated by alternative pathways that are not involved in Mz resistance, or they may bypass Mz detoxification or efflux pathways. Alternatively, improved nitro compounds may be activated by the same pathways that are suppressed in Mz resistance, but the residual activity of these pathways, which may not be completely shut down due to significant fitness costs (10), is sufficient to mediate adequate activation of the most potent new nitro compounds.

Our data suggest that incremental structural modifications, as frequently employed for lead compound optimization (37), may not be an optimal strategy for 5-NI drugs, as compounds with different and unpredictable structural features had desirable antimicrobial properties in vitro and in vivo. Although the precise mechanistic reasons for this counterintuitive observation remain to be determined, 5-NIs can be activated by different microbial pathways and probably target multiple microbial molecules (12, 14, 33) whose relative importance may depend on the compound and microbe. Because the specific target molecules of 5-NI drugs are not fully understood (33), a strategy focused on achieving effective whole-cell antimicrobial activity in the relevant systems may be the best choice for making rapid progress in developing new antimicrobials in the clinically indispensable 5-NI class.

### Materials and Methods

Six azido-alkyl-5NI heterocycles and 60 alkynes were synthesized de novo from commercially available starting materials, and 50 additional alkynes were purchased. Click reactions were performed with 100 mM each of azide and alkyne in t-BuOH: H₂O (2:1), 20 mM sodium ascorbate, and 5 or 10 mM CuSO₄ at 50° C. for 1-2 days (19). In vitro activity (EC50) against different isolates of Giardia and Trichomonas were determined with as single-step ATP assay and cytotoxicity (CC50) against human cells (HeLa) was determined with Alamar blue (6). Antibacterial activities were determined by OD600 after 24-48 h growth in culture. Quantitative structure-activity relationships were established using 1,666 chemical descriptors (E-Dragon 1.0) (38) and machine learning software (WEKA) (39). In vivo efficacy was assessed in adult C57BL/6 mice orally infected with G. lamblia GS/M (6). See S1 text for details.

### Example 2

### Clinical Importance of Giardiasis

The CDC category B pathogen, Giardia lamblia, is a major cause of parasitic diarrheal disease worldwide, with hundreds of millions of annual cases occurring in endemic and epidemic fashion (40-42). In the United States, Giardia is the most common cause of outbreaks of parasitic disease, with prevalence rates of 1-7%, depending on the population sampled (43). Infections are more frequent and severe in young children, particularly in day-care centers, and among travelers, hikers, and military personnel in the field (40, 43, 44). Infection is initiated by ingestion of cysts, which are shed in feces, can survive in water for months, and are resistant to many disinfectants (45). Fewer than ten cysts can establish human infection, making the parasite highly infectious and a credible accidental and bioterrorism threat to the safety of public water supplies (46, 478). Once ingested, flagellated, non-invasive trophozoites emerge from the cysts and colonize the upper small intestine where they attach to the epithelial lining, causing villus and brush border microvillus atrophy, and digestive enzyme deficiencies (41, 48-50). Half of all stool-positive Giardia infections are symptomatic with diarrhea, epigastric pain, nausea, and vomiting, which can cause malabsorption and malnutrition, especially in children. Acute giardiasis may disable patients for extended periods (51) and elicit protracted post-infectious syndromes (52), while chronic infection can lead to delayed development and impairment of cognitive skills in children (53).

### Significance of 5-Nitro Heterocylic Drugs in Giardia Treatment.

The synthetic 5-nitroimidazole (5NI), metronidazole (Mz), is highly active in vitro against Giardia, as well as Trichomonas vaginalis, Entamoeba histolytica and several important anaerobic bacterial pathogens (e.g. Helicobacter pylori). Mz has a long record of efficacy and safety in humans (54). It is typically given in three daily 250 mg oral doses for 5-10 days (15-30 total doses), and is effective in 80-95% of cases (55). Single-dose treatment is much less effective (20-60%) (56, 57). Major adverse effects are headache, nausea and unpleasant metallic taste, which together with inconvenient dosing can lead to poor compliance. Alternatives to Mz are the long-acting 5NI compounds, tinidazole and ornidazole, which can be given in a single 2 g oral dose and have similar efficacy (85-90%) as 7 days of Mz (58), but only tinidazole is available or used in the U.S. Giardia resistant to Mz are cross-resistant to the other two 5NI drugs (58). Non-5NI nitro drugs have been used against giardiasis, but they generally have lower efficacy. The 5-nitrothiazole, nitazoxanide, is given in two daily 500 mg doses for three days (6 total doses), but has slightly lower efficacy (70-80%) than 5NI drugs (16) and is also impacted by Mz resistance (19). The nitrofuran, furazolidone, is recommended for children, but also has lower efficacy (80-85%) than 5NI drugs and shows moderate cross-resistance to Mz (58). Among non-nitro antigiardials, albendazole exhibits 25-90% efficacy, depending on the dosing regimens (40, 55, 59), and can be used in combination with Mz (60). The antimalarial drug, quinacrine, is up to 90% effective against giardiasis (61, 62), but can have serious adverse effects and is not widely used for this purpose. Other drugs are active against Giardia, including auranofin, but their utility in treating giardiasis remains to be established (63). In sum, nitro drugs, particularly Mz, remain the most effective antigiardials and the current standard of treatment for giardiasis.

### Action Mechanisms of Nitro Drugs.

Nitro drugs are prodrugs whose microbial specificity is due to the strict requirement for reduction to toxic free radical intermediates by low redox potential reactions present only in the anaerobic target microbes. Giardia metabolism is fermentative, and electron transport proceeds in the absence of mitochondrial oxidative phosphorylation. However, the parasite is microaerotolerant (64) and can reduce 02 and thus protect the highly oxygen-sensitive, central metabolic enzyme, pyruvate:ferredoxin oxidoreductase (PFOR), and iron-containing ferredoxins (65, 66). PFOR decarboxylates pyruvate and donates electrons to ferredoxin, which in turn reduces other components in the electron transport chain and leads to ATP generation. Reduced ferredoxin can reduce the critical nitro group of Mz to toxic radicals which kill the parasite. Mz and other nitro drugs are specific due to their low redox potential (−415 mV for Mz), with ferredoxins having redox potentials around −430 mV in anaerobes (67). Importantly, the lowest electron couple in the human host, NADH/NAD, with a redox potential of −320 mV, cannot reduce Mz. Other reduction pathways, including nitroreductases and thioredoxin reductase, also reduce 5NI drugs in Giardia (68-71), although their relative importance in activating Mz and other nitro drugs is not clear. The radicals that result from nitro drug reduction form covalently bonded adducts on microbial target molecules, which leads to their inactivation. Several protein targets, including thioredoxin reductase and purine nucleoside phosphorylase, have been identified in Entamoeba (72), while the specific molecular targets of nitro drugs in Giardia remain unknown.

### Mz Resistance in Giardia.

In spite of the general efficacy of 5NI drugs, treatment failures in giardiasis are common (up to 20%), clinical resistance is proven, and in vitro resistance can be induced so that parasites grow in clinically relevant levels of Mz (58, 61, 63, 73). Importantly, Mz-resistant (MzR) Giardia exhibit cross-resistance to other prescribed 5NI drugs and nitazoxanide (58). Resistance has been elicited in vitro against all antigiardial drugs (73-76), demonstrating the need for developing new compounds to stay ahead of the parasite's ability to evade treatment. Prior studies (77) and new data (see below) indicate that side chain modifications of 5-nitro heterocyclic compounds can markedly improve antigiardial activity and overcome Mz resistance. This concept is well established for other, more advanced antibiotic classes. For example, the 4th generation cephalosporin, cefepime, has similar antimicrobial activity as earlier cephalosporins, but is less susceptible to lactamase-dependent resistance (78). Superior (“next-generation”) nitro drugs for the single-dose eradication of MzR and Mz-sensitive (MzS) giardiasis are developed by evaluating a comprehensive, newly synthesized library of >1,150 nitro compounds.

### Mz in the Treatment of Amebiasis.

The protozoan parasite Entamoeba histolytica, also a CDC Category B pathogen, infects ˜10% of the world's population. Like Giardia, it is transmitted by the fecal-oral route, but unlike Giardia, the parasite colonizes the colon and cecum, where it invades the mucosa and kills host cells, causing debilitating diarrhea and dysentery in adults (79). Systemic spread can lead to hepatic and potentially fatal brain abscesses (80). The energy metabolism of Entamoeba is also primarily anaerobic, rendering the organism susceptible to killing by 5NI drugs, especially Mz. Resistance to Mz related to increased superoxide dismutase has been described (81), yet Mz is the only drug approved for treatment of invasive amebiasis. For these reasons, development of new, more potent antimicrobials against Entamoeba is a high priority and an important secondary goal of the studies.

The studies facilitate successful drug development: 1. We have synthesized a comprehensive new library of >1,150 structurally diverse nitro drug candidates, which constitutes a powerful new resource for antimicrobial drug discovery. 2. Initial screening of the library indicates that it is highly likely that we identify new candidate drugs that are safe and effective against Mz-sensitive and -resistant Giardia in in vitro and in vivo. 3. We have assembled a unique set of drug-sensitive and -resistant syngeneic Giardia lines of both human pathogenic assemblages that represent the best available test system for developing new antimicrobials to overcome drug resistance. 4. The available nitro drug library, combined with our new drug development strategies, are readily applicable to other important pathogens susceptible to nitro antimicrobials, including E. histolytica, Trichomonas vaginalis, Trypanosoma cruzi, Helicobacter pylori, Clostridium difficile, Cryptosporidium parvum, Cyclospora, Blastocystis hominis, Gardneralla vaginalis and Mycobacteria. Several of these have shown a tenacious ability to develop resistance to Mz, indicating an urgent need for development of new therapeutics.

### Approach

Giardia is a major worldwide cause of diarrheal disease. Metronidazole (Mz) is the current treatment of choice for giardiasis, yet treatment failures occur in up to 20% of cases, and resistance to the drug develops in vitro and in vivo. In addition, standard treatment regimens for Mz are cumbersome (15-30 doses over 5-10 days) and prone to compliance failure. Tinidazole has a shortened dosing regimen, but is equally impacted by Mz resistance, while existing non-5NI drugs are generally not as effective as 5NI antigiardials. Our extensive preliminary data suggest that new nitro drugs are more active than Mz, can overcome Mz resistance, and are efficacious in animal models without acute toxicity (see below). Based on these promising results and the underlying clinical challenge, studies are done to achieve the following primary objective of developing a safe “next-generation” nitro antimicrobial drug candidate that is more potent than existing drugs against Mz-sensitive giardiasis, efficacious against Mz-resistant Giardia, and therapeutic in a single-dose oral regimen.

The following sections summarize our data in support of the rationale and technical feasibility of the project.

### Synthesis of Comprehensive New Nitro Drug Library.

Based on prior reports that side chain modifications can improve the activity of nitro antimicrobials (77, 82), we undertook a systematic effort to generate a structurally diverse library of new nitro heterocyclic compounds. We have synthesized 1,165 new compounds (20-50 mg each) with different structural features (FIG. 1). The compounds have as cores the heterocycles imidazole (classes A-J), pyrrole (K), thiazole (L) and furan (M). Side chain modifications were introduced in the heterocycles in the 1-position (A-C,K), 2-position (D-I,L,M), or 4-position (J). Linkage between the nitro heterocyclic core and the side chains were either direct (F) or through ethanyl (E), ethenyl (D), methylene triazole (G,M), ethylene triazole (A-C,H,J,K), aldoxim (I), or propanamide (L). Side chains were structurally diverse from simple linear alkyls to complex substituted multicyclic groups. The compounds were synthesized with a range of chemistries, including aldol condensation (83), Suzuki coupling (84), and cycloaddition (85). Conclusions: We have synthesized 1,165 new nitro heterocyclic compounds as candidate antimicrobials. Based on literature searches and consultation with key opinion leaders in the field, this number doubles or even triples the number of nitro compounds currently available anywhere, thus providing a powerful new resource for antimicrobial drug discovery and the basis for the studies.

### Example 3

### Superior In Vitro Antigiardial Activity of New Nitro Compounds

To begin evaluation of the nitro drug library, we first tested all compounds for activity against a representative Mz-sensitive G. lamblia strain, 713 (belonging to Assemblage A), using a 48 h growth and survival assay to determine the effective concentration that inhibits growth by 50% (EC50) (83). All structural groups had compounds superior to Mz, with percentages ranging from 4-100% and maximal improvements of up to 500-fold (FIG. 2A). In the entire library, 775 of the compounds (67%) were more active than Mz. Testing of a subset of 36 compounds against a second Mz-sensitive G. lamblia isolate, the Assemblage B strain GS/M, revealed a good correlation (r=0.57) of activities compared to the 713 strain, with 78% of compounds showing superiority over Mz against both lines (FIG. 2B). Conclusions: The new nitro drug library has 775 compounds with superior activity to Mz against a representative Giardia strain. The majority of a subset of compounds was active against two different strains.

### Synthesis of New Nitro Drug Library.

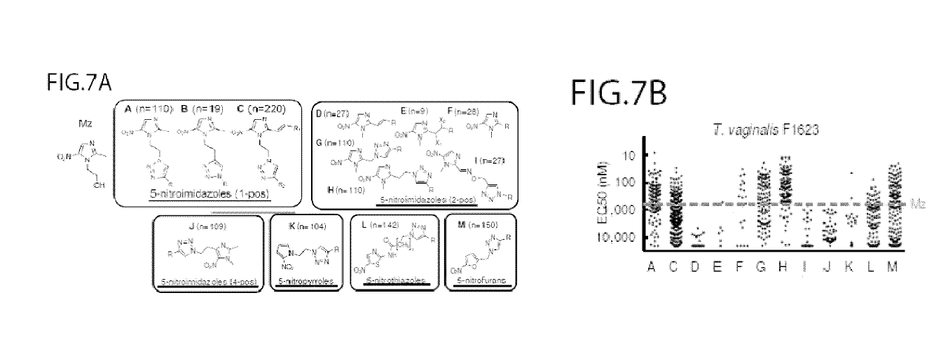

A new library of 1,165 structurally diverse nitro heterocyclic compounds of the indicated classes was synthesized. The number of individual compounds (n) in each class is shown in FIG. 7. Metronidazole (Mz) is shown for comparison.

### Establishment of Stable Mz-Resistant Giardia Lines.

Development of new nitro antimicrobials that combat Mz resistance critically depends on the availability of Mz-resistant (MzR) Giardia lines. Because such lines have not been established from patients who failed Mz therapy, we have acquired or newly developed a set of laboratory-generated MzR lines from genetically diverse, Mz-sensitive (MzS) parental G. lamblia isolates (WB, 106, 713 and 1279). All lines are axenic, grow well in culture, and show resistance in form of 4-40 fold increases in EC50 for Mz and tinidazole (Tz) (Table 1) (58, 63). Expressed differently, Mz and Tz had only 2.5-25% of “residual activity” (RA) in the resistant lines, as calculated by the formula 100%×(EC50 in MzS/EC50 in MzR). This calculation emphasizes the relative impact of resistance on drug activity (which is key for treating both MzS and MzR giardiasis). Importantly, drug resistance was maintained over several freeze-thaw cycles, extended growth without Mz selection, and in vivo passage through mice (58), demonstrating that the phenotypes are stable and likely genetic in origin. Conclusions: Our unique collection of syngeneic pairs of MzS and stable MzR Giardia lines constitutes a key tool for the nitro drug development.

TABLE 1
Development of MzR Giardia lines. The activity of Mz and tinidazole (Tz) against
four syngeneic pairs of MzS and MzR G. lamblia lines was tested.
Residual activity (RA) against the MzR lines was calculated by the formula:
RA = 100% × (EC50 in MzS/EC50 in MzR).

| | EC50 (μM) | | | | | | | | | | | |
|---|---|---|---|---|---|---|---|---|---|---|---|---|
| | G. lamblia WB | | | G. lamblia 106 | | | G. lamblia 713 | | | G. lamblia 1279 | | |
| Drug | MzS | MzR | RA | MzS | MzR | RA | MzS | MzR | RA | MzS | MzR | RA |
| Mz | 2.9 | 80 | 4 | 3.6 | 78 | 5 | 2.0 | 50 | 4 | 4.4 | 17 | 26 |
| Tz | 0.5 | 20 | 3 | 1.1 | 24 | 5 | 1.6 | 7.1 | 23 | — | — | — |

RA: Residual drug activity (%)

### New Nitro Compounds can Overcome Mz Resistance.

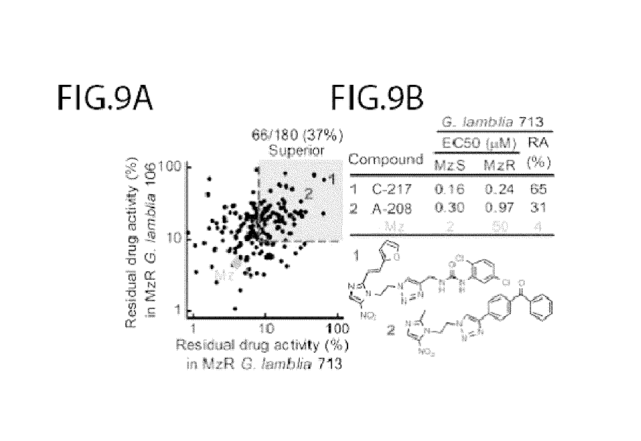

We then tested a subset of nitro compounds from the library against two syngeneic pairs (713 and 106) of MzS and MzR Giardia lines. Of 180 compounds, 66 (37%) showed >2-fold greater residual activity than Mz in both MzR lines (highlighted in grey shade, FIG. 9A). Importantly, only a small fraction (<10%) of all compounds in the set had detectable cytotoxicity in human HeLa cells. Examples of the best compounds, along with their antigiardial activities and relative activity, are shown in FIG. 9B. Conclusions: We have identified multiple new nitro compounds that overcome Mz resistance in two different MzR Giardia lines. None of the compounds had acute toxicity in human cells. Identification of these superior compounds demonstrates the feasibility of the preclinical drug development.

### Efficacy of New Nitro Drugs in Animal Models of Giardiasis.

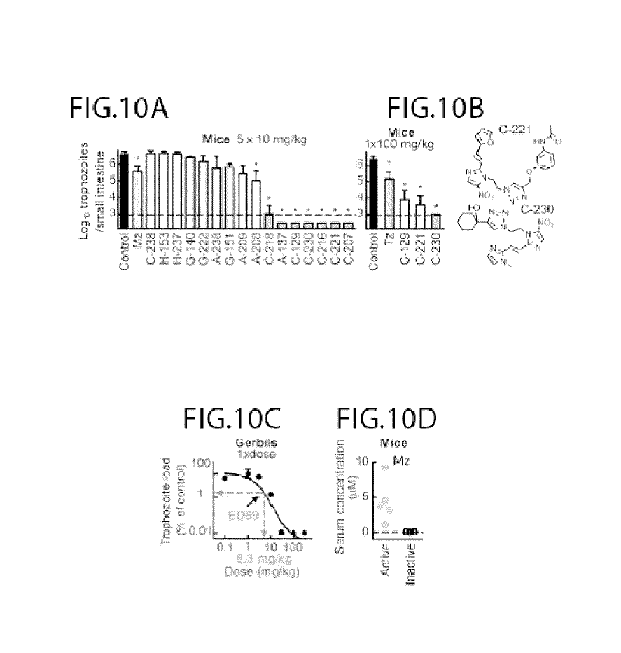

We next tested the new nitro compounds in vivo. Adult mice were infected with G. lamblia GS/M, which was derived from a patient with chronic, severe diarrhea, and caused diarrheal disease upon experimental human infection (8). Four days after infection, mice were given five oral drug treatments over 2.5 days, and trophozoites were counted in the small intestine. We found that 7 of 16 (44%) structurally representative compounds were more efficacious than Mz, while none showed acute toxicity in mice (FIG. 10A, left panel). At least three compounds were efficacious against giardiasis with a single oral dose in mice (FIG. 10A, right panel; with examples shown in FIG. 10B) and gerbils (FIG. 10C). Furtheimore, active compounds generally had higher plasma levels than inactive compounds, but considerable variation was observed (FIG. 10D). The active compounds are shown as solid dark dots in the left region of the figure with the label “active” below the dots. Conclusions: New 5NI compounds are efficacious and non-toxic in mouse and gerbil models of giardiasis.

### Example 4

### Lead Indication and Broad-Spectrum Activity

Our project involves developing a new candidate nitro drug that is effective against Mz-sensitive and Mz-resistant giardiasis in a single-dose oral regimen. Despite this specific goal, we realize that existing nitro drugs have multiple antimicrobial activities and several clinical indications, and that the RFA announcement “emphasizes development of broad-spectrum therapeutics.” In fact, our preliminary screens show that many of the new nitro compounds have improved broad-spectrum activity. However, we consider the focus on one therapeutic indication as necessary for regulatory and practical reasons, because a strong IND application requires a lead indication and because a single therapeutic goal streamlines the study design and maximizes the chances of success. Accordingly, the systematic identification of the top compounds from the new nitro drug library are primarily guided by antigiardial activity and safety. Nonetheless, for the top compounds that emerge from the screens with similar activity and safety profiles, we consider their potential broad-spectrum activity against other pathogenic target microbes, particularly E. histolytica, but also others (e.g. T. vaginalis), which are treated with nitro drugs. Although such data are not required for an IND application, inclusion of this information in a comprehensive preclinical portfolio of the top candidate drug enhance the utility and commercial potential of that drug, because a drug approval for the lead indication facilitates relatively time- and cost-effective expansion of clinical testing to other indications.

### Drug Development Strategy.

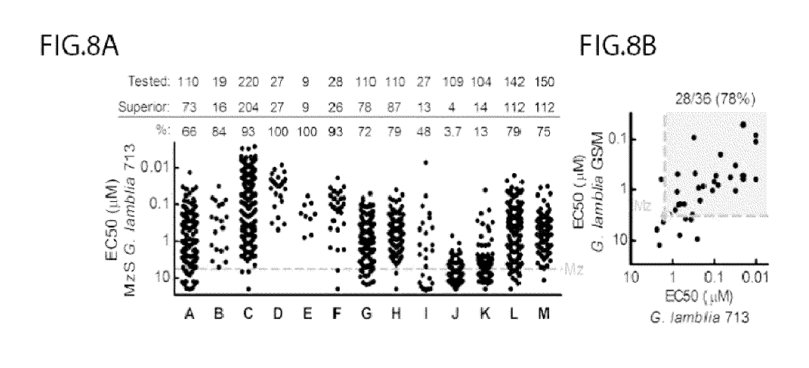

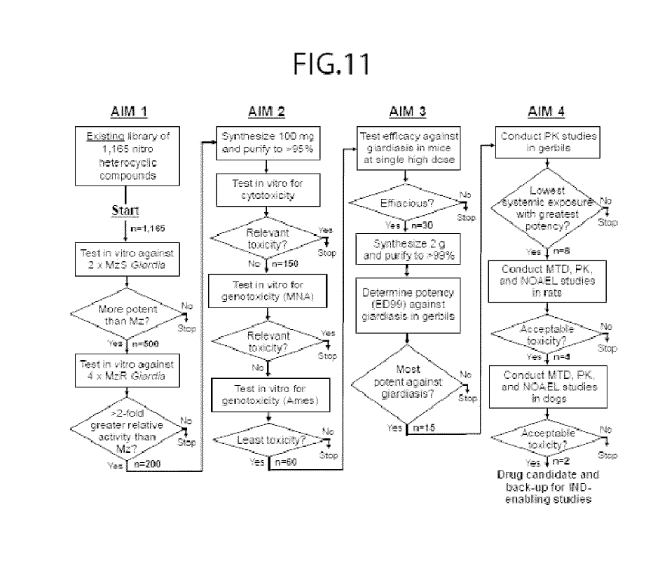

We have generated a new library of 1,165 structurally diverse nitro drugs (FIG. 1) and determined in initial screens that many of the compounds have superior antigiardial activity and can overcome different forms of Mz resistance (FIG. 8,9). Based on these data, a full evaluation of the entire existing library yields compounds that have the desired activity and safety profiles in vitro and in vivo, and are strong candidates for definitive preclinical evaluation in preparation of an IND application. Accordingly, a comprehensive evaluation strategy (FIG. 11) can be performed that systematically proceeds from in vitro screens (Aims 1 and 2) to in vivo evaluation (Aims 3 and 4).

FIG. 11 shows the drug development strategy. The project can develop a new antimicrobial that is effective against Mz-sensitive (MzS) and Mz-resistant (MzR) giardiasis at a single oral dose. The starting point is a newly constructed, comprehensive nitro drug library with documented superior activities against Giardia. The flow chart in FIG. 11 depicts the major experiments and decision points for the systematic evaluation of the entire library. Estimates for the number of compounds (n) at different stages of the project are depicted.

In Aim 1, we determine potency against diverse MzS and MzR Giardia lines in vitro, and apply defined screening criteria to select the most active compounds. The surviving compounds are evaluated in Aim 2 for cytotoxicity and genotoxicity in vitro. Compounds that pass the in vitro safety screens are prioritized based on their ability to overcome different forms of Mz resistance, and a subset is tested in Aim 3 for single-dose efficacy and potency in suitable animal models of giardiasis. Active compounds are then investigated in Aim 4 for their pharmacokinetic and toxicological profiles in vivo to identify the compounds with the least systemic exposure, greatest potency, and best safety profile. At project end, we can develop two new candidate antimicrobials, a top compound for subsequent ND-enabling studies and a second compound as a back-up if concerns arise about the top compound in further pre-IND evaluation.

### Example 5

### Comprehensive In Vitro Testing of Antigiardial Activity

We have synthesized a comprehensive new nitro drug library (FIG. 7A), whose initial evaluation has identified many compounds with superior activities against selected MzS and MzR Giardia lines (FIG. 8,9). The activities against T. vaginalis F1623 of these compounds are listed in FIG. 7B and the activity of Mz is shown as the dashed line in FIG. 7B. However, the compounds have not yet been evaluated for broad activity against diverse MzS and MzR Giardia strains, which is important for further preclinical development. Therefore, in this Aim we conduct comprehensive in vitro testing against an array of drug-sensitive and -resistant Giardia strains (Table 1) (58, 63). In further studies, we test in vitro activity against the anaerobic protozoan pathogen, E. histolytica, as broad-spectrum activity against several enteric parasites is a desirable feature that can expand indications and help to drive commercial development of a new nitro drug. Later, we also evaluate whether resistance development can occur against the most promising new nitro drugs.

FIG. 8 shows the antigiardial activity of some nitro compounds. FIG. 8A shows the activity of all library compounds was tested against the Mz-sensitive (MzS) G. lamblia strain 713. The dashed line in FIG. 8A depicts Mz activity. FIG. 8B shows a subset of compounds was tested against a second Giardia lines, 106, and the data were related to those in the 713 line. The shaded region highlights compounds more active than Mz against both strains.

FIG. 9 shows the drug activity against MzR Giardia. Activities of 180 selected nitro compounds were determined against MzR lines of G. lamblia 713 and 106, and are expressed as percentages of residual activity (RA) relative to the parental MzS cells. Superior compounds are defined as those with >2× greater residual activity than Mz and are highlighted in A (light grey background). Two examples are labeled as 1 and 2, and detailed information on their antigiardial activities and structures is provided in FIG. 9B.

### In Vitro Activity Against MzS Giardia.

Nitro compounds are tested for antigiardial activity in two rounds, first against two MzS lines and then against a broader set of MzR lines. As sensitive lines, we employ the G. lamblia strains WB and GS/M, which are the best studied representatives of genetic assemblages A and B, respectively (86). New antimicrobials must be active against Giardia of both of these human-pathogenic assemblages (86). Serial dilutions of test compounds (from 20 μM to 1 nM), or Mz as a positive control, are made in Giardia growth medium in 96-well plates, using our robotic liquid handling system (Beckman Biomek 3000). Solvent alone serves as a control. Trophozoites (2,000/well) are added, and plates are incubated anaerobically (Anaerocult system, Merck) at 37° C. for 48 h. Because quantitative data are needed for compound evaluation (most compounds are expected to be active, so differences in potency are critical), we determine growth and viability with a quantitative ATP assay at the end of the incubation period, as we have reported before (58, 63, 83). The BacTiter Microbial Cell Viability Assay (Promega) is a rapid luminescence assay of ATP levels in live microbial cells, which is based on a firefly luciferase-catalyzed reaction of ATP with luciferin. The assay is not affected by components in the growth media (e.g. serum, bile, yeast extract) or by nitro drugs, and thus permits single-step analysis of ATP directly in the microtiter wells (83) Luminescence is plotted against drug concentrations, and EC50 values (effective concentrations that inhibit growth by 50%) are derived by numeric interpolation. Testing proceeds in two steps: All compounds are tested in strain WB, and compounds more potent than Mz in that strain are tested against the second strain, GS/M. Compounds with greater activity than Mz in both MzS strains are pursued.

### In Vitro Activity Against MzR Giardia.

Next, we test the surviving compounds against four diverse MzR Giardia lines (Table 1) (58, 63). These lines were derived in the laboratory from four MzS Giardia isolates (WB, 713, 106, 1279) of different geographic origins (United States, Europe, Australia) representing both relevant assemblages, A and B. The lines exhibit stable Mz resistance and display distinct forms of resistance, as indicated by differential responses to different nitro drugs (83) and implicated mechanisms of resistance (68, 70, 87). Furthermore, the lines are syngeneic pairs for which we can determine drug potencies (EC50) in the sensitive and resistant cells, allowing us to calculate the residual drug activity in MzR cells relative to the matching MzS cells with the formula: 100%×(EC50 in MzS/EC50 in MzR). Clinically drug-resistant Giardia isolates have been used in short-term animal studies (88), but axenic lines have not been reported to date. Thus, our diverse drug-resistant lines represent a powerful (and the best available) test system for developing new antimicrobials to overcome Mz resistance in Giardia.

Activity testing is done as described above, using all four MzR lines and the two parental MzS lines 106 and 1279 (the other two parental lines, 713 and WB, were already tested in our preliminary studies or are in the first round of testing). For each compound, we determine the residual activity in MzR cells relative to the parental MzS cells in the syngeneic pairs. Compounds are selected that exhibit a >2-fold improvement, compared to Mz, in residual activity against the resistant lines. To illustrate this selection criterion, the EC50 values for Mz are 2 μM for the sensitive 713 line and 50 μM for the resistant 713 line, which means that the relative activity of Mz is 100%×(2 μM/50 μM)=4% in MzR compared to MzS cells (FIG. 9B). Compound E-217 has EC50s of 0.16 μM in MzS and 0.24 μM in MzR cells, which represents a residual activity of 65% (FIG. 9B) and thus meets the criterion (because 65% activity is >2-fold greater than the residual Mz activity of 4%). In contrast, another compound, E-226 (not shown), displays EC50s of 0.008 and 0.4 μM (residual activity 2.1%) and does not meet the selection criterion (because 2.1% is not >2-fold greater than the 4% activity of Mz). The rationale for this selection criterion is based on comparison of Giardia isolates from patients with successful or failed Mz therapy, which suggested that a >2-fold improvement in drug potency compared to drug-resistant cases was correlated with successful therapy (88). Moreover, our focus on relative compound potency and on minimizing the impact of cross-resistance, rather than absolute EC50, is rooted in the clinical need to treat both sensitive and resistant infections without prior knowledge of the resistance status of the infecting Giardia (drug sensitivity testing is not done for Giardia), and the likelihood that a future clinical trial could not be readily stratified for drug susceptibility. Testing again proceeds in a stepwise fashion with the matched pairs of MzS and MzR lines. After testing of the first Giardia pair (WB), we select the compounds that pass the activity criterion and begin testing the second pair, and so on. Compounds that pass the criteria in all four pairs are advanced.

In vitro activity against Entamoeba.

A secondary goal of the project is identification of new antimicrobials with broad-spectrum activity against other anaerobic parasites, particularly the protozoan category B pathogen E. histolytica. Mz is the treatment of choice for amebiasis, but resistance can occur (81) and treatment alternatives are very limited (80). Therefore, we test for amebicidal activity of those compounds that meet the activity criteria for giardiasis, and prioritizew compounds that have “dual activity” against Entamoeba. Testing is done in 96-well plates under anaerobic conditions with cellular ATP content as a read-out, as described for Giardia. We use the laboratory-adapted, axenic and virulent HMI:INNS isolate of E. histolytica, as we have in the past (89-91). We also have access to non-axenized clinical E. histolytica isolates (92) from our long-term collaborator, Dr. Sharon Reed at UCSD, although their use is complicated by variable growth and the presence of undefined bacteria in the cultures.

### 4. Resistance Development in Giardia Against New Nitro Drugs.

New nitro antimicrobials are specifically selected to overcome existing forms of Mz resistance, but it is possible, in principle, that Giardia can develop resistance against new drugs. Importantly, this potential is not a deterrent to new drug design, but rather a justification. Furthermore, it can be integrated into drug development, since the risk of resistance may vary between drugs. As a first step towards assessing resistance risk for new drugs, we begin growing MzS cells (WB) in increasing but sublethal concentrations of the most promising compounds, as we have described before (58, 73). To accelerate resistance development, some of the cultures are briefly exposed to UV light to promote mutagenesis before drug selection (58). Mz and tinidazole are used in parallel for comparison. We perform these assays for a fairly small number of compounds, so the experiments are performed once we have identified the 15 top compounds with the most promising in vitro activity (this Aim), safety profiles in vitro (Aim 2), and greatest efficacy in vivo (Aim 3). Compounds against which resistance develops more slowly than Mz are given higher priority for further development.

Based on our preliminary screens (FIG. 8,9), we anticipate that 40-70% of all library compounds are more potent than Mz against both MzS Giardia strains to be tested. Of these, 30-40% are expected to meet the criteria for overcoming Mz resistance (i.e., >2-fold improvement in residual activity in resistant over sensitive cells compared to Mz) in the syngeneic pairs, so we estimate that some 200 compounds in the 1,165 compound library fulfill all antigiardial activity criteria. If we find more, we select the 200 most active and structurally diverse compounds. Selected compounds advance to cytotoxicity and genotoxicity testing in vitro (Aim 2). Testing of amebicidal activity is done after Giardia testing, although the results may affect later selection of the top candidates. Preliminary data show that 35 of 110 new nitro compounds were more active than Mz against E. histolytica. However, negative Entamoeba activity data precludes further pursuit of a compound, since our primary goal is the development of superior therapeutics against giardiasis.

### Example 6

### Cytotoxicity and Mutagenicity Testing In Vitro

New antimicrobials must pass extensive toxicological tests to be drug candidates for ultimate human use. Assessment of genotoxicity is particularly important for nitro antimicrobials, as they possess an aromatic nitro group that is considered a “structural alert”, and they have been suspected as mutagens (93). Most FDA approved nitro drugs are mutagenic in the Ames test due to bacterial nitroreductases (94). However, extensive clinical evaluation over decades has not shown relevant mutagenicity in humans (54), indicating that nitro drugs are generally safe. Nonetheless, early genotoxicity testing remains especially important for nitro drug development. Therefore, the selected compounds are next subjected to comprehensive cytotoxicity and genotoxicity testing in vitro in cooperation with our industrial partner SRI International.

### 1. Cytotoxicity in Mammalian Cells.

All nitro compounds (up to 200) that meet antigiardial activity criteria in Aim 1 are tested for cytotoxicity in mammalian cells. Compounds are re-synthesized at a 100 mg level, purified to >95% by chromatography, and dried under nitrogen (our library compounds were initially synthesized at 20-50 mg levels and were mostly left in the original reaction solvents, which does not interfere with antigiardial activity testing, but could confound toxicity evaluation). After resuspension in the universal solvent DMSO, cytotoxicity are tested in human HeLa epithelial cells (83) and mouse 3T3 fibroblasts (89). Test compounds, or Mz and tinidazole as controls, are serially diluted in growth media and added to the cells in 96-well plates. After 48 h, cell viability are determined with an Alamar Blue dye reduction assay (83). CC50 values (i.e., the cytotoxic concentrations that reduce cell viability by 50%) are derived from the concentration-response curves and used to calculate selectivity ratios relative to the antigiardial EC50 values. Compounds with selectivity ratios of >100, or no detectable toxicity at the highest testable concentration (as limited by solubility), in both HeLa and 3T3 cells are advanced to genotoxicity testing.

### 2. Genotoxicity in Mammalian Cells.

Genotoxicity is first tested in mammalian cells, because the results are likely to be more informative for nitro compound selection than those from bacterial assays (see below). We employ a micronucleus assay with cytokinesis block, which detects chromosomal aberrations by the appearance of micronuclei in dividing cells after one cell division (95). Cytochalasin-B is added to inhibit cell division, thus allowing cells that have completed one nuclear division to be readily recognized by their binucleate morphology. Human primary lymphocytes and several cell lines (including CHO cells) have been validated in this assay (96), but we utilize CHO cells, as their use is easier and more cost-effective. Cells are exposed for 3-24 h to different concentrations (1 mM to 100 nM) of the test compound, or Mz and tinidazole as controls, in culture medium in the presence or absence of metabolic activation (with S9 rat liver extract). After exposure, cells are rinsed and incubated with Cytochalasin B for an additional 8-24 h, after which they are fixed and stained with Hoechst 33342 DNA dye and a fluorescent cytoplasmic counterstain (97). A total of 1,000 binucleated cells per concentration are analyzed for micronuclei. In addition, 200 cells are evaluated for their proliferative status by counting the number of mono-, bi-, tri- and tetra-nucleated cells and calculating the nuclear division index as an indicator of cytostatic drug effects. Compounds that show no significant genotoxic activity in the micronucleus assay at the highest possible test concentration (as limited by solubility, which we evaluate microscopically) are prioritized. Compounds with detectable genotoxicity, but with a >100-fold higher minimum toxic concentration (MTC) than their antigiardial EC50, may also be retained for further testing, but receive a lower priority.

### 3. Bacterial Genotoxicity (Ames Test).

Bacterial genotoxicity tests, particularly the Ames test, are technically simple, widely accepted, and generally required for an IND application (98). However, they often yield false positive results for nitro drugs due to ubiquitous bacterial nitroreductases (94), making them less valuable for compound selection than mammalian cell tests. Nonetheless, we perform bacterial tests on all nitro compounds that pass the micronucleus assays to gain additional safety information and for later inclusion in an IND application. We screen for mutagenic activity using the plate incorporation procedure with two Salmonella tester strains (TA98, TA100) (99). In addition, we use nitroreductase-deficient mutants of TA98 and TA100, generated by our SRI collaborators, as they can help to interpret any positive test results from the standard strains. Mz, tinidazole, known chemical mutagens, and solvent alone is included as controls. A wide range of doses are tested up to 5 mg/10 cm plate or the lowest precipitating dose for compounds with limited solubility, and compounds are incubated in the presence and absence of an induced rat-liver metabolic activation system (S9). After overnight incubation, test plates are compared with control plates for revertant counts and the condition of the background bacterial lawn. Compounds are deemed mutagenic when the mean number of revertant colonies on the test plates exceeds the mean solvent control counts by >2-fold. Dose dependence is considered in evaluating mutagenic responses. Cytotoxicity is estimated by a decrease in the number of revertant colonies, clearing or absence of the background bacterial lawn growth, formation of pinpoint non-revertant colonies, or absence of bacterial growth.

Our prior studies (73) and preliminary experiments have indicated that <10% of nitro compounds display measurable cytotoxic activity, so we anticipate that most of the 200 compounds to be tested survive these screens. The outcome of the mammalian genotoxicity assays is harder to predict, but our initial screens suggest that 30-60% of the compounds are negative in these tests. Thus, combined with the cytotoxicity data, we expect to identify 50-100 compounds that pass all microbial activity and mammalian cell toxicity tests in vitro. Of these, we select up to 60 compounds with the highest activity towards MzS and MzR cells, the lowest toxicity, and the greatest structural diversity for subsequent in vivo testing. Furthermore, the Ames tests mostly yield positive results, which would not disqualify any compounds from further development (because bacteria can non-specifically activate most nitro drugs), but are useful information for an IND application. If we find compounds that are negative in the micronucleus assays and Ames tests, we prioritize them for further studies.

### Example 7

### Efficacy and Potency Testing in Rodent Models of Giardiasis

Any new antimicrobial drug candidate is designed to kill the target microorganism in vitro, but demonstration of in vivo activity is necessary to make the candidate promising for further development towards a human drug. Past experience shows that new drug candidates are often initially active in vitro, but fail later in development to be efficacious in vivo (100). With this in mind, we have placed special emphasis on timely integration of comprehensive in vivo testing in our drug development strategy. Preliminary studies show that about half of the tested compounds were active against giardiasis in rodent models (FIG. 4A), supporting the excellent prospects of new nitro compounds. Nonetheless, in vivo activity cannot be taken for granted and needs to be evaluated for the most promising candidates as they advance through the screening process. Moreover, our objective to develop a drug candidate that is active in a single-dose regimen poses an additional challenge. We focus on drug candidates with intrinsic in vivo activity from the start, rather than try to impart in vivo efficacy by focused modifications of molecules with adequate in vitro but no initial in vivo activity. Our approach is more likely to yield candidates with robust properties that can survive the entire preclinical evaluation process. Thus, in this Aim we investigate the in vivo activity of the compounds that meet in vitro activity criteria (Aim 1) and have passed the in vitro cytotoxicity and genotoxicity screens (Aim 2).

### 1. Efficacy Against Giardiasis in Mice.

We begin compound evaluation for in vivo efficacy in a mouse model of giardiasis, using the pathogenic G. lamblia GS/M strain that was shown to cause diarrheal disease in human volunteers (47). Mice are a suitable and cost-effective infection model for initial screens (63, 83). Adult mice (6-8 week old C57BL/6J females) are infected with 106 GS/M trophozoites by oral gavage. Trophozoites, rather than cysts, is used for inoculation, because they are the disease-causing forms of the parasite that need to be targeted by new antimicrobials. In addition, they are easier to obtain than cysts on a routine basis and they initiate infection with less variation than cysts, thereby reducing the number of animals needed for significant data. The inoculum is 10- to 50-fold lower than peak infection levels in mice (63, 101), so drug efficacy is tested against actively proliferating parasites. After 4 days to allow establishment of the infection, mice are given a single oral dose of one of the up to 60 test compounds that were selected, re-synthesized, and purified in sufficient quantity in Aim 2. Controls receive Mz, tinidazole or vehicle only. We use a single high dose of 100 mg/kg in groups of 8 animals to determine rapidly and with the smallest meaningful number of animals if a compound has significant in vivo efficacy. This dose is the lowest single Mz dose that eradicates giardiasis in at least half of infected mice (not shown). Compounds are generally given in aqueous solution, but if solubility is limiting, we make a crude drug suspension with a slightly viscous methylcellulose-containing (0.5%) buffer (which has worked well for us). Two days after drug administration, live trophozoites in the small intestine are enumerated microscopically (63, 83). Mean and SD are calculated from the log 10-transformed numbers. Mice without detectable parasites (i.e., <103/small intestine) are considered cleared. Differences to the vehicle (100%) controls are evaluated for significance by Mann-Whitney rank sum test. Compounds that significantly reduce trophozoite load at the 100 mg/kg dose compared to vehicle controls are retained. About 50% (i.e., 30 of 60 to be tested) of the compounds are active in mice (see FIG. 10A), but if we find more, we selected the top 30 most efficacious compounds for subsequent in vivo tests in gerbils.

### 2. Potency Against Giardiasis in Gerbils.

We next determine compound potency against giardiasis in Mongolian gerbils (Meriones unguiculatus). They can be readily infected with diverse G. lamblia strains (58, 102) and are currently the best and most relevant animal model for human giardiasis because of the extended course of infection (6-8 weeks) and the infection-induced pathophysiologic changes reminiscent of diarrheal disease in humans (103-105). In addition, gerbils represent a second animal species (beyond mice) for efficacy testing, which broaden the applicability of the mouse data and are helpful for an IND application. These tests also provide us with potency information for the interpretation of pharmacokinetic studies (Aim 4). Despite their utility, it must be noted that gerbils require more resources than mice, making them a better choice for more advanced evaluations of a smaller number of selected compounds, rather than for the initial in vivo efficacy screens of larger numbers (for which mice are ideal, see above).

We first synthesize and purify ˜2 g amounts of the test compounds (up to 30) and purify them to >99% by chromatography and repeated recrystallization. Adult (10 week old) male gerbils are then infected by oral gavage with 106 trophozoites of G. lamblia WB (63) and left alone for 10-14 days to allow maximal host colonization. A single dose of the test compound are given to groups of 4 animals and two days later, live trophozoite numbers are determined in the small intestine. Mz, tinidazole (which is effective in humans at a single dose, but is fully impacted by cross-resistance) (58), and vehicle alone serve as controls. Because it is important for compound evaluation in these experiments to determine drug potency, rather than merely efficacy at a single high dose, we establish dose/response curves. We start with a slightly higher dose (300 mg/kg) than was efficacious in adult mice and then progressively lower the dose (e.g. 100, 30, 10, 3 mg/kg) until efficacy is lost. Percentages of trophozoite counts in treated vs. untreated animals are plotted against drug doses, and ED99 (i.e., the effective dose that causes 99% mean reduction in trophozoite load) and the minimum eradication dose (i.e., the lowest dose that leads to undetectable infection after 2 days) are derived by numeric interpolation and expressed in mg/kg. Moreover, we perform confirmatory studies with the minimum eradication dose after 21 days to ensure that infection is indeed permanently eradicated. Afterwards, we select the best 15 compounds that are most potent (i.e., lowest ED99, because lower drug doses are generally better than higher doses). In parallel to potency testing, we monitor animals for body weight and general clinical parameters to provide a first assessment of adverse drug effects (detailed toxicity testing are done over extended dose ranges in Aim 4).

In preliminary studies, about half of the tested nitro compounds have so far proven active in mouse models (FIG. 10A), suggesting that 30 of the 60 compounds to be tested are efficacious against giardiasis in mice. Because we have not seen any acute toxicity of our nitro compounds in mice (even when administered repeatedly), we do not expect major problems at this stage. The potency evaluation in gerbils is likely to confirm that most compounds efficacious in mice are also active in gerbils, although their exact potencies may show differences. Upon Aim completion, we expect to have identified 15 nitro compounds that fulfill all in vitro activity and safety criteria, and are most active against giardiasis in two rodent infection models.

If we find an unexpectedly low number (<5) of acceptable compounds, we would have two general options: 1. More extensive evaluation or improved formulation of existing compounds, or 2. Additional modification of the best nitro compounds. For option 1, we could include in vitro screens for drug stability (e.g. acidic conditions) or optimize formulation to improve in vivo efficacy. For example, carriers such as cyclodextrins or polyamidoamine dendrimers can help to dissolve poorly water-soluble compounds and increase local or systemic bioavailability (106, 107). For option 2, we would consider additional syntheses to modify a small number of the most promising nitro drugs in an incremental fashion, similar to what our research group has done before (83, 108). Sets of new compounds would then be evaluated as described above.

### Example 8

### Pharmacokinetic and Toxicological Evaluation In Vivo

Oral candidate drugs must typically achieve plasma levels that permit activity at systemic target sites, but this situation may not readily apply to giardiasis since the parasites reside exclusively in the intestinal lumen where drugs are present soon after oral uptake. In fact, compounds that are efficacious with lower plasma levels are preferable over similarly active compounds with higher plasma levels, since lower systemic exposure reduces the risk of adverse effects. The importance of this concept is demonstrated by poorly absorbable oral antibiotics such as rifaximin and neomycin (109), and are applied here to nitro drug development. Its general feasibility is supported by our preliminary finding of marked differences in plasma levels between different in vivo active compounds (FIG. 10B). This Aim determines the pharmacokinetic properties of the 15 most promising nitro compounds and relate them to their in vivo potency. The top 8 compounds are then be subjected to comprehensive toxicological testing in rats and dogs in preparation for IND-enabling studies.

### 1. Pharmacokinetic Studies in Gerbils.

To enable pharmacokinetic studies, we first develop, together with our industrial partner SRI International, bioanalytical methods to assay levels of the unmodified (non-radiolabeled) compounds in plasma (110, 111). Method development is done for two assay steps: 1. Extraction of the analyte from the biological matrix by protein precipitation, liquid/liquid extraction, or solid phase extraction; and 2. Chromatographic separation of the analyte from interfering endogenous materials in the extract, followed by detection by liquid chromatography and/or mass spectrometry (LC-MS/MS). Chromatography conditions (e.g. column selection, mobile phase composition, gradient profile) are chosen so the analyte elutes at an optimal retention time, and with adequate peak shape and reproducibility. Usually a reverse-phase C8 or C18 column of small dimensions (e.g. 2×50 mm) is used to minimize run times. For LC-MS/MS, we employ multiple reaction monitoring (MRM) for analyte detection with high sensitivity and specificity. We also select an internal standard that elutes at, or close to, the retention time of the analyte to correct for detection variability. Detection sensitivities of 5-50 ng/ml (or 10-100 nM for an average 500 Da nitro drug) can usually be achieved with assay optimization, which is 10- to 100-fold below the peak plasma levels we have observed so far (FIG. 10B).

To relate pharmacokinetic properties to antigiardial potency in the same animal model, we orally administer the test compounds (already synthesized in Aim 3 with >99% purity), or Mz and tinidazole as controls, at the minimum eradication dose (determined in Aim 3) to adult male gerbils (left uninfected with Giardia to simplify the studies). Blood (200 μl) are collected at different times (0.5, 1, 2, 4, 8, and 20 h post oral dose) by saphenous vein bleeding. Three gerbils are bled for each time point, but animals are not bled more than twice. Compound plasma levels are determined by LC-MS/MS and used to derive key pharmacokinetic parameters, including peak plasma concentration (Cmax), peak time (Tmax), terminal half-life (t1/2), and area under the curve (AUC). Particularly important for our drug evaluations are Cmax and AUC, which represent peak and total plasma exposure of a drug, respectively, because we want to minimize systemic drug exposure without compromising efficacy. It must be emphasized that these studies provide an initial assessment of the pharmacokinetic profile of candidate drugs after oral dosing, but are not designed to generate the comprehensive information required in later ND-enabling studies (such as clearance, oral bioavailability, and volume of distribution). Of the 15 test compounds, we select the top 8 with the lowest Cmax or AUC (i.e., lowest systemic exposure) in absolute terms and relative to their potency (ED99).

### 2. Toxicological Studies in Rats.

As a first step in the comprehensive toxicological evaluation in vivo, we conduct single-dose range-finding toxicity studies in rats at SRI international to determine the maximum tolerated dose (MTD) of the test compounds. MTD information is useful for estimating the therapeutic index of MTD over therapeutic dose (i.e., minimum eradication dose in gerbils), and is required for an IND application. Rats are most commonly used for toxicological studies in rodents, and extensive comparative data sets are available. Efficacy against giardiasis is be evaluated in rats, because we already have such data from two other rodent models (mice and gerbils) and a reliable rat model of giardiasis has not been established.

We first produce and purify 100-200 g of each of the up to 8 test compounds (which is within the synthetic capacity range of the laboratory of our TSRI collaborators) under non-GLP conditions. Purification to >99% are done by chromatography and repeated recrystallization. Adult male rats (Sprague-Dawley, 3 per group) then receive a single oral dose of one of the 8 test compounds and are observed for four days for mortality, any clinical signs of morbidity, and body weight. If any mortality occurs, the study is terminated at the respective dose. If all animals survive, they are held for a 7-day washout period, and then re-challenged with a higher drug dose. Dosing begins at the minimum eradication dose observed in gerbils, and is progressively escalated by 3-, 10-, 30-, and 100-fold up to a maximum of 1,500 mg/kg (112). If no toxicity is observed at the highest dose, we stop the study as successful. The MTD is derived from the data upon study completion. Additional rats are given a minimum eradication dose or MTD of the test compound, and drug plasma levels are determined up to 24 h, as discussed above, to ensure that levels resemble those after a therapeutic dose in gerbils or are proportional to the increase in dose, respectively. Furthermore, we collect bone marrow after 24 and 48 h from the single-dosed animals and analyze micronucleus formation (see Aim 2) as a measure of acute genotoxicity in vivo (113).

All compounds with acceptable MTD (>10× minimum eradication dose) are then tested in a 7-day repeat dosing toxicity study at SRI International. We determine the maximum tolerated dose and no observed adverse effect level (NOAEL) of each of the test compounds after 7 daily oral administrations to male and female rats. Although our primary dosing goal is single-dose administration, repeat exposure increases the stringency of the toxicological tests. Repeat dosing follows a similar strategy to single dosing, starting with the minimum eradication dose in gerbils and subsequent escalation. Clinical parameters are determined daily, and blood is collected at 4 and 7 days to determine drug levels and general disease markers (e.g. liver transferases, bilirubin, WBC counts, RBC sedimentation rate) in a comprehensive hematology and clinical chemistry panel. After 8 days, rats are euthanized and autopsied for histological analysis of the major internal organs (e.g. liver, lungs, kidneys, heart, brain, lymph nodes, spleen, and intestinal tract). The results are used to select the top four, least toxic candidates to advance to dog studies.

### 3. Toxicological Studies in Dogs.

Toxicity testing in two animal models, one rodent and one non-rodent, is mandatory for an IND application. After completion of the rat tests, we evaluate up to four drug candidates in dogs to determine their MTD and pharmacokinetics. Pairs of beagle dogs (1 male, 1 female) receive a single oral dose at three dose levels (low, mid, and high, as suggested by the rat data) and are observed for 4 days for morbidity, clinical signs of adverse effects, and body weight. Plasma is obtained at different times after dosing to assay drug levels. After 4 days, blood is collected and analyzed with test panels for hematology, clinical chemistry, and coagulation. If no toxicity is observed at the tested doses, additional doses may be given to extra dogs to determine the MTD. This is a survival study and animals are returned to the colony and assigned to a 7-day repeat-dosing toxicity study after a 2-week washout period. Among the test compounds with favorable toxicity and pharmacokinetic profiles in the single-dose tests, we then select the top two compounds with the highest MTD in rats and dogs, and the lowest minimum eradication dose and systemic exposure in gerbils, and determine their NOAEL after 7 daily oral administrations to male and female beagle dogs, following a similar strategy as in rats. The results of this dog study are used to select one candidate to proceed to IND-enabling studies. The other compound serves as a back-up in case toxicity of the top compound is found to be unacceptable in subsequent IND-enabling studies.

Pharmacokinetic studies are only done on compounds with proven efficacy against giardiasis in mice and gerbils, so we anticipate interpretable data for each of the up to 15 compounds to be tested. Our preliminary finding of 5-fold plasma level variation between different active compounds (FIG. 10B) suggests that testing of greater numbers identifies drugs whose levels may be as much as 10- or 20-fold lower than those of the most absorbed drugs, although the relationship of plasma levels to antigiardial potency remains to be established. It is also uncertain whether efficacious nitro drugs can be found with little or no plasma exposure, since our initial data indicate that very low or undetectable plasma levels are associated with loss of efficacy (FIG. 10B). Nonetheless, the goal of combining maximal potency with minimal systemic drug exposure would remain valid even if zero systemic exposure cannot be achieved. The results of the toxicological studies in rats and dogs are least predictable, although the widespread safe clinical use of nitro antimicrobials over decades (54) would suggest that a favorable outcome is likely. We have quantitative information for the key in vivo parameters (e.g. ED99, MTD, NOAEL), so we can establish a numerically-based priority ranking to guide selection of the top two compounds for IND-enabling studies.

### Example 9

### IND-Enabling Studies for Giardiasis

Upon completion of the proposed studies, we expect to have one new top drug candidate and one back-up compound for the single-dose treatment of MzS and MzR giardiasis that are superior to existing antigiardial drugs. An important step in further drug development is to arrange a pre-IND meeting with FDA experts and discuss a proposal for IND-enabling studies. All of the data to be generated in this project constitute critical information for the proposal, and is used as preliminary data for an IND application. Further details are discussed in the Product Development Plan.

### Other Therapeutic Indications.

An important aspect of the project is that we have synthesized and characterize a large number of new, structurally diverse nitro heterocyclic compounds, which have great potential to be active against many important human pathogens, including E. histolytica, Trichomonas, Trypanosoma, and mycobacteria. Screening of diverse compounds that have passed initial safety screens against these pathogens is likely to yield important new activities that could be exploited for drug development outside the current project.

### Mechanisms of Action of New 5NI Drugs.

Our antimicrobial drug development strategy identifies compounds with superior in vitro and in vivo activity and favorable pharmacokinetic and toxicity profiles. The studies are not designed to elucidate the underlying mechanisms of action (MOA), which are likely to be complex and have not been truly defined for any nitro antimicrobial (the mechanisms of nitro drug activation are generally known, but the relevant molecular targets of the activated drugs are not clear for most microbes, including Giardia). Such studies would be beyond the scope and resources of this project. Furthermore, knowledge of specific drug targets is not necessary for successful drug development, and has historically often not been the major driving force behind effective new drugs for clinical use. Nonetheless, understanding the target of any new drug can be valuable, as the target may remain valid even if a particular drug candidate fails during drug development. We have recently developed new chemical approaches for identifying the microbial targets of nitro antimicrobials, and would consider applying them to defining the action mechanisms of the best compounds that emerge from the project. The strategy utilizes nitro drugs with a small “chemical handle” that does not impact activity, but facilitates isolation and identification of adducted target molecules. Activity assays and genetic knock-down technology can then be used to determine the impact of drug adduction on target function and the consequences for microbial survival. Identification of critical targets could be a starting point for a new project to design even more active drugs of the nitro class, or perhaps other chemical classes, against those targets.

### Example 10

### Synthesis and Structural Characterization

### 1.1 General

All chemical reagents and solvents were purchased from commercial suppliers and used without further purifications. ¹H, ¹³C NMR spectra were obtained on Varian Inova 400 MHz or Bruker 500 MHz spectrometers. The chemical shifts (δ) were expressed in parts per million (ppm) relative to residual: CHCl₃ (δH7.26 ppm), CDCl₃ (δC77.0 ppm), CH₃SOCH₃ (δH2.50 ppm) and CD₃SOCD₃ (δC39.43 ppm). NMR acquisitions were performed at 295 K unless otherwise noted. Abbreviations are: s, singlet; d, doublet; t, triplet; q, quartet, brs, broad singlet. High resolution mass spectrometry was performed on an Agilent ES-TOF instrument at The Scripps Center for Metabolomics and Mass Spectrometry. Reactions were monitored by LCMS analysis (Hewlett-Packard Series 1100, ESI MS) eluting with 0.1% trifluoroacetic acid in H₂O and 0.05% trifluoroacetic acid in CH₃CN and/or TLC chromatography using Merck TLC Silica Gel 60 F254 plates and visualized by staining with cerium molybdate (Hanessian's Stain) or by absorbance of UV light at 254 nm. Crude reaction mixtures were purified by column chromatography using Merck Silica Gel 60 as stationary phase. The purity of all synthesized alkynes was ≧90% based on the LCMS analysis. The purity and conversion of all “Click chemistry” reaction mixtures were ≧80% based on the LCMS analysis. Selected products for in-vivo studies were prepared in pure form and characterized by ¹H, ¹³C NMR and HRMS (ESI TOF).

### 1.2. Synthesis of Azides (A-F)

### 1.2.1. Synthesis of Azide A

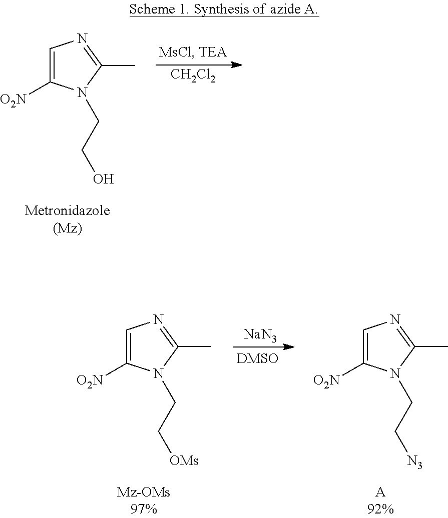

### 2-(2-Methyl-5-nitro-1H-imidazol-1-yl)ethyl methanesulfonate (Mz-OMs)

A solution of methanesulfonyl chloride (27.0 mL, 40.0 g, 349 mmol, 1.2 equiv.) in CH₂Cl₂ (50 mL) was added dropwise into stirred, cooled (=10° C.) suspension of metronidazole (50 g, 292 mmol), TEA (61 mL, 44.3 g, 439 mmol, 1.5 equiv.) in CH₂Cl₂ (250 mL) (114, 115). After addition the reaction was stirred at r.t. for 4 h then evaporated. The solid residue was washed with H₂O and dried on vacuum to give white solid (71 g, 97%). ¹H NMR (400 MHz, d₆-DMSO) δ=8.06 (s, 1H), 4.65 (t, 0.1=5.0 Hz, 2H), 4.55 (t, J=5.0 Hz, 2H), 3.15 (s, 3H), 2.46 (s, 3H). ¹³C NMR (100 MHz, d₆-DMSO) δ=151.6, 138.3, 133.0, 68.4, 45.0, 36.6, 13.9.

### 1-(2-Azidoethyl)-2-methyl-5-nitro-1H-imidazole (A)

A suspension of Mz-OMs (71 g, 285 mmol) and NaN₃ (22.2 g, 341 mmol, 1.2 equiv.) in DMSO (600 mL) was stirred at 70° C. for 4 h. Subsequently, the mixture was cooled, diluted with H₂O (1 L) and brine (2 L) and extracted using CHCl₃ (4×300 mL). Combined organic layers were washed with H₂O (2×500 mL), dried over MgSO₄, filtered and evaporated to give white solid of A (51.7 g, 92%). ¹H NMR (400 MHz, CDCl₃) δ=7.95 (s, 1H), 4.41 (t, J=5.6 Hz, 2H), 3.75 (t, J=5.6 Hz, 2H), 2.52 (s, 3H). ¹³C NMR (100 MHz, CDCl₃) δ=151.2, 138.1, 133.3, 50.8, 45.4, 14.4.

### 1.2.2. Synthesis of Azide B

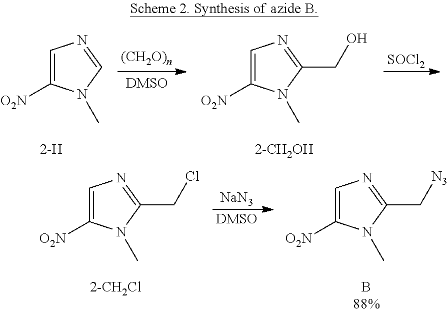

### (1-Methyl-5-nitro-1H-imidazol-2-yl)methanol (2-CH₂OH)

The title compound was prepared according to the literature procedure (116). ¹H NMR (400 MHz, d₆-DMSO) δ=8.00 (s, 1H), 5.67 (t, J=6.0 Hz, 1H), 4.58 (d, J=6.0 Hz, 21.1), 3.92 (s, 3H). ¹³C NMR (100 MHz, d₆-DMSO) δ=152.3, 139.2, 131.4, 56.0, 33.2. ESI MS (MeOH) for [M+H]⁺=158.2 Da.

### 2-(Chloromethyl)-1-methyl-5-nitro-1H-imidazole (2-CH₂Cl)

The title compound was prepared according to the literature procedure (117). ¹H NMR (400 MHz, CDCl₃) δ=7.93 (s, 1H), 4.67 (s, 2H), 4.03 (s, 3H). ¹³C NMR (100 MHz, CDCl₃) 5=147.1, 139.6, 131.7, 36.1, 33.8. ESI MS (MeOH) for [M+H]⁺=176.2 Da.

### 2-(Azidomethyl)-1-methyl-5-nitro-1H-imidazole (B)

A mixture of 2-CH₂Cl (3 g, 17.1 mmol), NaN₃ (1.22 g, 18.8 mmol, 1.1 equiv.) in DMSO (20 mL) was stirred at r.t. overnight. Subsequently, the mixture was diluted with EtOAc (150 mL) and washed with brine (5×25 mL). The organic layer was passed through short silica-gel pad and evaporated to give B as a brown oil (2.76 g, 88%). ¹H NMR (400 MHz, CDCl₃) δ=7.94 (s, 1H), 4.49 (s, 2H), 3.98 (s, 3H). ¹³C NMR (100 MHz, CDCl₃) 5=146.6, 139.7, 131.8, 46.7, 33.6.

### 1.2.3. Synthesis of Azide C

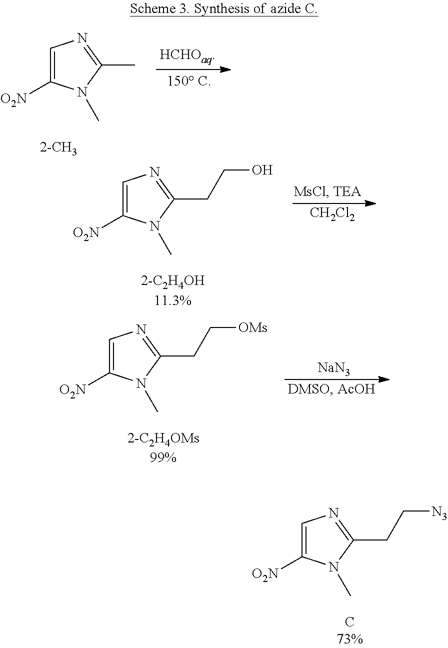

### 2-(1-Methyl-5-nitro-1H-imidazol-2-yl)ethanol (2-C₂H₄OH)

A mixture of 2-CH₃ (40 g, 283 mmol) and 37% formaldehyde solution in H₂O (140 mL, 1.86 mol, 6.6 equiv.) in high-pressure resistant sealed glass tube was heated at 150° C. for 12 h, then cooled down and carefully opened. The brown solution was stirred with EtOAc (1 L) at 60° C. for 15 min. The organic layer was separated and evaporated. The residue was purified by column chromatography (silica-gel, EtOAc→5% MeOH in EtOAc) to give a yellowish solid that was additionally purified by crystallization from CH₃CN (20 mL) (5.5 g, 11.3%). H NMR (400 MHz, d₆-DMSO) δ=8.02 (s, 1H), 4.84 (t, J=5.4 Hz, 1H, —OH), 3.85 (s, 3H, N-Me), 3.77-3.73 (m, 2H), 2.91 (t, J=6.0 Hz, 2H). ¹³C NMR (100 MHz, d₆-DMSO) δ=152.4, 138.7, 132.4, 58.8, 33.1, 30.6.

### 2-(1-Methyl-5-nitro-1H-imidazol-2-yl)ethyl methanesulfonate (2-C₂H₄OMs)

To a suspension of 2-C₂H₄OH (2 g, 11.7 mmol) and TEA (1.8 mL, 1.3 g, 12.9 mmol, 1.1 equiv.) in anhydrous CH₂Cl₂ (30 mL), methanesulfonyl chloride (1 mL, 1.48 g, 12.9 mmol, 1.1 equiv.) was added. The mixture was stirred at r.t. for 1 h and H₂O (30 mL) was added. The organic layer was dried over MgSO₄, filtered and evaporated to give a yellow solid (2.9 g, 99%). The crude product was used in the next step without purification. ESI MS (MeOH) for [M+H]⁺=250.1 Da.

### 2-(2-Azidoethyl)-1-methyl-5-nitro-1H-imidazole (C)

To a mixture of 2-C₂H₄OMs (7 g, 28 mmol) and AcOH (8 mL, 8.4 g, 140 mmol, 5 equiv.) in DMSO (60 mL), NaN₃ (5.5 g, 84 mmol, 3 equiv.) was added. The mixture was stirred at r.t. for 1 day, and then diluted with H₂O (30 mL) and EtOAc (100 mL). The organic layer was washed with brine (5×25 mL), dried over MgSO₄, filtered and evaporated. The residue was purified by column chromatography (silica-gel, EtOAc) to give an orange solid (4 g, 73%). ¹H NMR (400 MHz, CDCl₃) δ=7.97 (s, 1H), 3.94 (s, 3H), 3.83 (t, J=6.6 Hz, 2H), 2.96 (t, J=6.6 Hz, 2H). ¹³C NMR (100 MHz, CDCl₃) 5=149.9, 132.4, 125.6, 48.6, 33.2, 27.4. ESI MS (MeOH) for [M+H]⁺=197.4 Da.

Elimination of the methanesulfonic acid takes place and 1-Methyl-5-nitro-2-vinyl-1H-imidazole is a major side product. Addition of AcOH reduces amount of the vinyl side-product.

### 1.2.4. Synthesis of Azide D

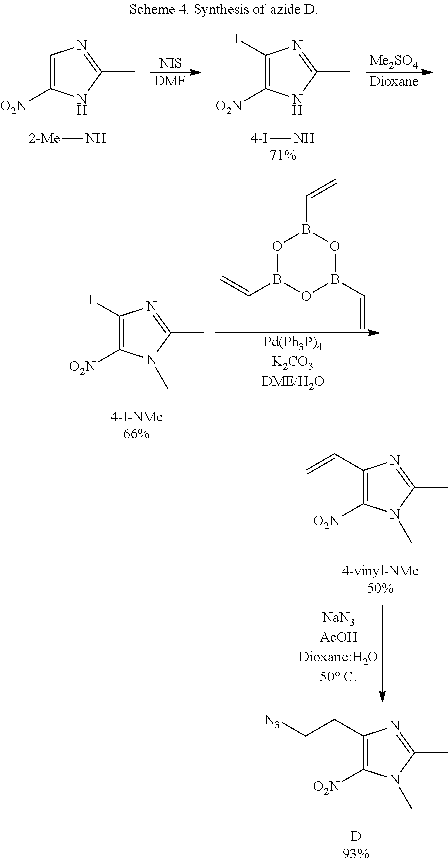

### 4-Iodo-2-methyl-5-nitro-1H-imidazole (4-I-NH)

The title compound was prepared using a similar literature procedure as for the bromination reaction (118). To a stirred warm (55° C.) solution of 2-Me-NH (45 g, 354 mmol) in anhydrous DMF (150 mL), NIS (87.5 g, 389 mmol, 1.1 equiv.) was added in one portion. After addition, the temperature was increased to 80° C. and the reaction was carried out for 4 h. Subsequently, the reaction was cooled and poured into H₂O (2 L). A yellow solid was filtered, washed with 10% Na₂S₂O₃ solution, H₂O and dried on high vacuum (63.5 g, 71%). ESI MS (MeOH) for [M+H]⁺=254.1 Da.

### 4-Iodo-1,2-dimethyl-5-nitro-1H-imidazole (4-I—NMe)

A mixture of 4-I—NH (63.5 g, 251 mmol) and Me₂SO₄ (31.6 g, 23.7 mL, 251 mmol) in 1,4-dioxane (600 mL) was stirred under reflux for 3 h and then evaporated. The resulting yellow oil was dissolved in H₂O (330 mL) and neutralized with saturated NaHCO_{3(aq)}. Yellow solid was filtered and washed with 1 M NaOH to give the product as a yellow solid (44 g, 66%). ¹H NMR (400 MHz, d₆-DMSO) δ=3.80 (s, 3H), 2.40 (s, 3H). ¹³C NMR (100 MHz, d₆-DMSO) δ=152.0, 139.5, 92.6, 34.4, 13.5. ESI MS (MeOH) for [M+H]⁺=268.2 Da. The product contains approx. 15% of the 5-iodo-1,2-dimethyl-4-nitro-1H-imidazole isomer. The structure of 4-I—NH was confirmed by comparison with the literature NMR spectra (119).

### 1,2-Dimethyl-5-nitro-4-vinyl-1H-imidazole (4-vinyl-NMe)

The title compound was prepared using a similar literature procedure (120). To a mixture of 4-I—NMe (17.7 g, 66.4 mmol) in degassed DME (520 mL), Pd(PPh₃)₄ (7.67 g, 6.6 mmol, 10% mol.) was added and the reaction was stirred under N₂ at r.t. for 20 min Subsequently, K₂CO₃ (9.15 g, 66.4 mmol), H₂O (160 mL) and vinylboronic anhydride pyridine complex (16 g, 66.4 mmol) were added. The reaction was stirred under reflux under N₂ for overnight, then evaporated to the final volume (˜100 mL) and extracted using Et₂O (2×300 mL). The combined organic layers were washed with 1 M HCl (4×50 mL). The aqueous layers were filtered, saturated with solid NaCl and neutralized with 2 M NaOH, and the aqueous mixture was transferred to separatory funnel and extracted using Et₂O (5×100 mL). The organic layers were dried over MgSO₄ and filtered. The product crystallized from Et₂O, yellowish solid (5.5 g, 50%). ¹H NMR (400 MHz, CDCl₃) δ=7.33-7.25 (m, 1H), 6.31 (dd, J=17.2, 1.6 Hz, 1H), 5.57 (dd, J=17.2, 1.6 Hz, 1H), 3.84 (s, 3H), 2.44 (s, 3H). ¹³C NMR (100 MHz, CDCl₃) δ=148.8, 141.6, 126.6, 121.8, 33.8, 14.1. ESI MS (MeOH) for [M+H]⁺=168.4 Da.

### 4-(2-Azidoethyl)-1,2-dimethyl-5-nitro-1H-imidazole (D)

To a mixture of 4-vinyl-NMe (2 g, 12 mmol), NaN₃ (2.34 g, 36 mmol, 3 equiv.) in 1,4-dioxane:H₂O (3:1, 80 mL), TEA (5.05 mL, 3.64 g, 36 mmol, 3 equiv.) and AcOH (6.86 mL, 7.2 g, 120 mmol, 10 equiv.) were added. The mixture was stirred at 50° C. for 2 days and then cooled. The reaction was neutralized with solid NaHCO₃ and evaporated to the final volume of ˜20 mL. The remaining solution was diluted with H₂O (100 mL) and extracted using EtOAc (2×100 mL). The combined organic layers were washed with brine, dried over MgSO₄, filtered and evaporated to give a red oil (2.35 g, 93%). ¹H NMR (400 MHz, CDCl₃) δ=3.85 (s, 3H), 3.65 (t, J=6.8 Hz, 2H), 3.21 (t, J=6.8 Hz, 2H), 2.43 (s, 3H). ¹³C NMR (100 MHz, CDCl₃) δ=148.3, 143.7, 49.0, 33.9, 29.1, 14.0. ESI MS (MeOH) for [M+H]⁺=211.2 Da.

CAUTION! Any experiments that may result in the formation of hydrazoic acid (HN₃) should be performed in a well-ventilated fume hood and behind a blast shield. Sodium azide should not be mixed with strong acids.

The product slowly decomposes at r.t. and should be stored at +4° C.

### 1.2.5. Synthesis of Azide E

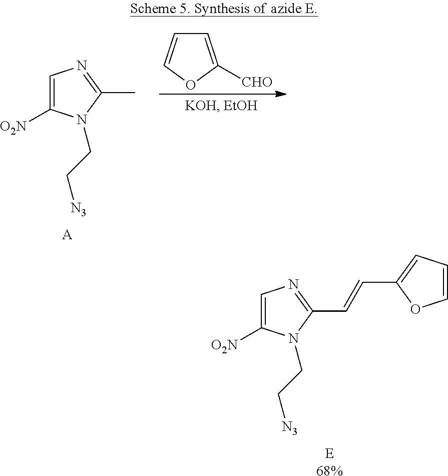

### (E)-1-(2-Azidoethyl)-2-(2-(furan-2-yl)vinyl)-5-nitro-1H-imidazole (E)

A mixture of A (4 g, 20.4 mmol), 2-furaldehyde (2.35 g, 24.5 mmol, 1.2 equiv.) and KOH (85%, 1.3 g, 20.4 mmol) in EtOH (20 mL) was stirred at 60° C. for one day. After cooling, the mixture was diluted with H₂O (200 mL) and a brown-yellow solid was filtered. Crude product was dissolved in EtOAc, passed through a short silica-gel pad and evaporated to give a yellow-red solid (3.8 g, 68%). ¹H NMR (400 MHz, CDCl₃) δ=8.10 (s, 1H), 7.64 (d, J=15.6 Hz, 1H), 7.48 (s, 1H), 6.82 (d, J=15.6 Hz, 1H), 6.58 (d, J=3.2 Hz, 1H), 6.49-6.47 (m, 1H), 4.58 (t, J=5.6 Hz, 2H), 3.77 (t, J=5.6 Hz, 2H). ¹³C NMR (100 MHz, CDCl₃) δ=151.5, 150.6, 144.3, 138.2, 135.1, 126.6, 114.1, 112.5, 109.0, 50.8, 44.6. ESI MS (MeOH) for [M+H]⁺=275.2 Da.

### 1.2.6. Synthesis of Azide F

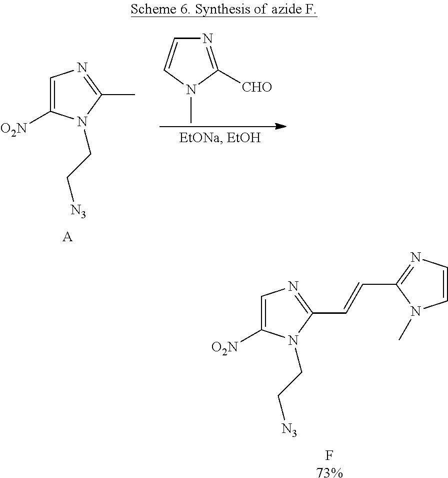

### (E)-1-(2-Azidoethyl)-2-(2-(1-methyl-1H-imidazol-2-yl)vinyl)-5-nitro-1H-imidazole (F)

To a solution of A (2.97 g, 15.1 mmol) in EtOH (15 mL), EtONa (2.55 g, 37.5 mmol, 2.5 equiv.) was added. The mixture was stirred at r.t. for 10 min and 1-methyl-1H-imidazole-2-carbaldehyde (2 g, 18.2 mmol, 1.2 equiv.) was added. The reaction was carried out at 60° C. for 2 h and then cooled and diluted with H₂O (100 mL). The mixture was extracted using EtOAc (2×100 mL) and the combined organic layers were dried over MgSO₄, filtered and evaporated. The residue was purified by column chromatography (silica-gel, EtOAc→10% MeOH in CHCl₃) to give an orange solid that was additionally purified by crystallization from EtOAc (3.2 g, 73%). ¹H NMR (400 MHz, d₆-DMSO) δ=8.28 (s, 1H), 7.69 (d, J=15.2 Hz, 1H), 7.39 (d, J=15.0 Hz, 1H), 7.31 (s, 1H), 7.07 (s, 1H), 4.69 (t, J=5.4 Hz, 2H), 3.82 (t, J=5.4 Hz, 2H), 3.79 (s, 3H). ¹³C NMR (100 MHz, d₆-DMSO) δ=149.9, 143.5, 138.6, 135.0, 129.3, 124.2, 123.5, 113.7, 50.4, 44.5, 32.5. ESI MS (MeOH) for [M+H]⁺=289.2 Da.

### 1.3. List and Synthesis of Alkynes for Training Set Compounds

### 1.3.1. List of Alkynes 101-163

The following alkynes were used to synthesize triazole compounds for the training set. Of these, alkynes 101-137 were newly synthesized, while alkynes 138-163 used in the synthesis were obtained from commercial sources.

TABLE 1.3a
Synthesized Alkynes (101-137)

| | |
|---|---|
| 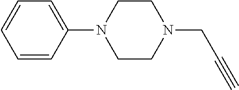 | 101 |
| 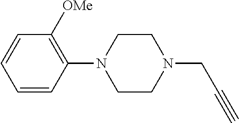 [SMILES: C#CCN1CCN(c2ccccc2OC)CC1] | 102 |
| 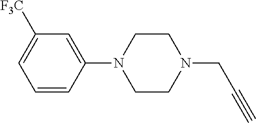 [SMILES: C#CCN1CCN(c2cccc(C(F)(F)F)c2)CC1] | 103 |
| 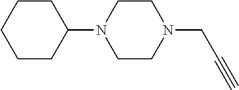 | 104 |
| 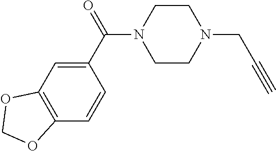 [SMILES: C#CCN1CCN(C(=O)c2ccc3c(c2)OCO3)CC1] | 105 |
| 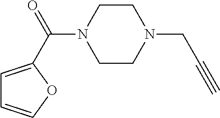 [SMILES: C#CCN1CCN(C(=O)c2ccco2)CC1] | 106 |
| 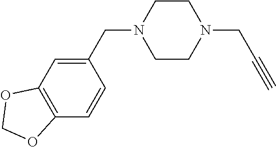 [SMILES: C#CCN1CCN(Cc2ccc3c(c2)OCO3)CC1] | 107 |
| 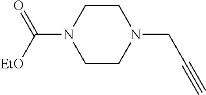 | 108 |
| 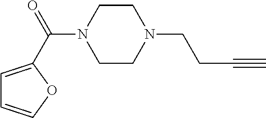 [SMILES: C#CCCN1CCN(C(=O)c2ccco2)CC1] | 109 |
| 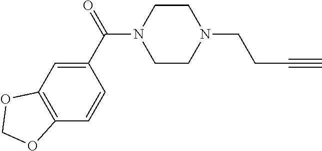 [SMILES: C#CCCN1CCN(C(=O)c2ccc3c(c2)OCO3)CC1] | 110 |
| 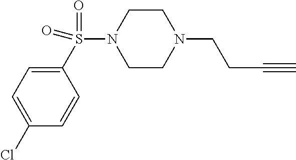 [SMILES: C#CCCN1CCN(S(=O)(=O)c2ccc(Cl)cc2)CC1] | 111 |
| 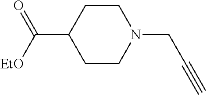 | 112 |
| 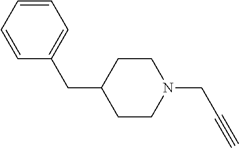 [SMILES: C#CCN1CCC(Cc2ccccc2)CC1] | 113 |
| 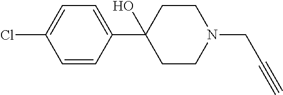 [SMILES: C#CCN1CCC(O)(c2ccc(Cl)cc2)CC1] | 114 |
| 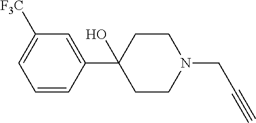 [SMILES: C#CCN1CCC(O)(c2cccc(C(F)(F)F)c2)CC1] | 115 |
| 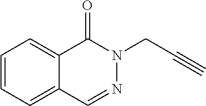 | 116 |
| 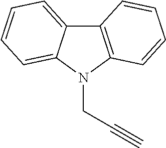 [SMILES: C#CCn1c2ccccc2c2ccccc21] | 117 |
| 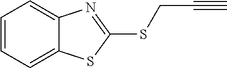 | 118 |
| 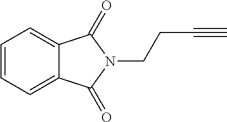 | 119 |
| 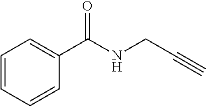 | 120 |
| 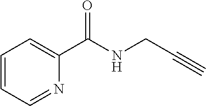 | 121 |
| 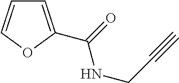 | 122 |
| 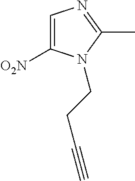 | 123 |
| 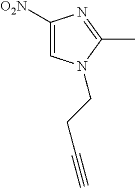 | 124 |
| 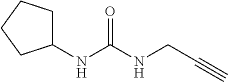 | 125 |
| 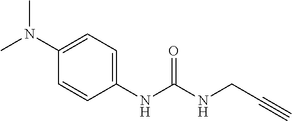 [SMILES: C#CCNC(=O)Nc1ccc(N(C)C)cc1] | 126 |
| 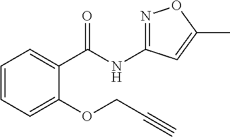 [SMILES: C#CCOc1ccccc1C(=O)Nc1cc(C)on1] | 127 |
| 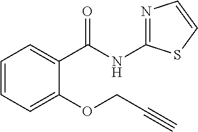 [SMILES: C#CCOc1ccccc1C(=O)Nc1nccs1] | 128 |
| 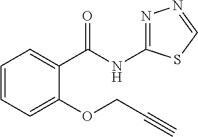 [SMILES: C#CCOc1ccccc1C(=O)Nc1nncs1] | 129 |
| 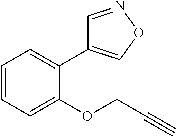 | 130 |
| 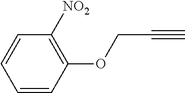 | 131 |
| 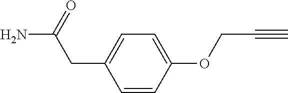 | 132 |
| 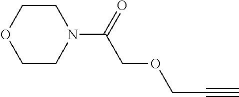 | 133 |
| 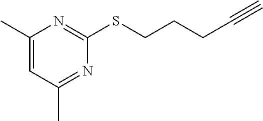 | 134 |
| 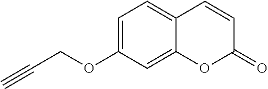 | 135 |
| 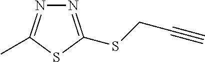 | 136 |
| 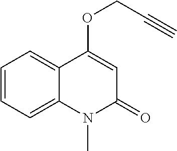 [SMILES: C#CCOc1cc(=O)n(C)c2ccccc12] | 137 |

TABLE 1.3b
Commercially available alkynes (138-163) used in the synthesis

| | |
|---|---|
| 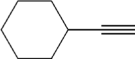 | 138 |
| 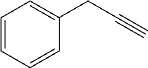 | 139 |
| 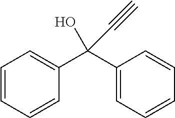 [SMILES: C#CC(O)(c1ccccc1)c1ccccc1] | 140 |
| 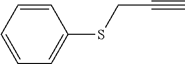 | 141 |
| 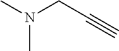 | 142 |
| 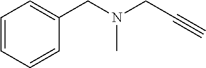 | 143 |
| 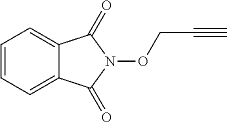 | 144 |
| 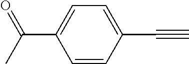 | 145 |
| 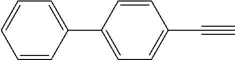 | 146 |
| 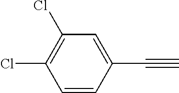 | 147 |
| 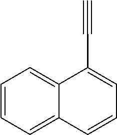 | 148 |
| 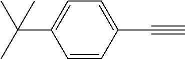 | 149 |
| 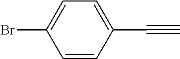 | 150 |
| 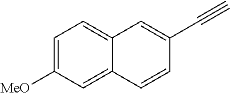 | 151 |
| 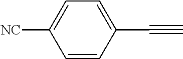 | 152 |
| 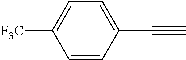 | 153 |
| 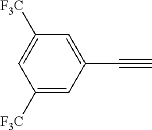 [SMILES: C#Cc1cc(C(F)(F)F)cc(C(F)(F)F)c1] | 154 |
| 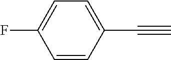 | 155 |
| 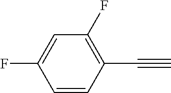 | 156 |
| 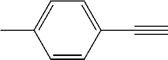 | 157 |
| 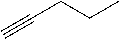 | 158 |
| 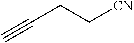 | 159 |
| 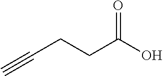 | 160 |
| 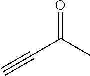 | 161 |
| 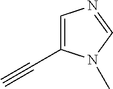 | 162 |
| 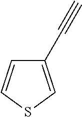 | 163 |

### 1.3.2. Synthesis of Alkynes 101-137

Compounds 117 (121), 118 (122), 119 (123), 120 (124), 122 (124), 131 (125), 135 (126), 137 (127) were prepared according to published procedures. Alkynes 102, 103, 107, 116, 121, 133 were described before but without general synthetic procedures (128).

### Synthesis of Alkynes 101-104, 108

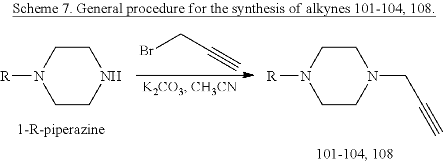

General Procedure:

To a mixture of an appropriate N-substituted piperazine (32 mmol) and anhydrous K₂CO₃ (4.4 g, 32 mmol) in anhydrous CH₃CN (50 mL), propargyl bromide (80% solution in toluene, 3.6 mL, 4.8 g, 32 mmol) was added. The mixture was stirred overnight under reflux, and then filtered and evaporated. The residue was purified by column chromatography (Hexanes:EtOAc).

### Synthesis of Alkynes 105-106

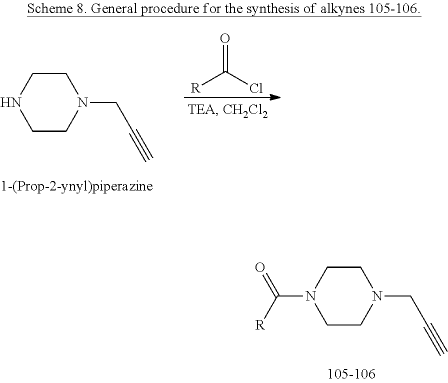

General Procedure:

To a mixture of 1-(prop-2-ynyl)piperazine (129) (1.25 g, 10.1 mmol) and TEA (1.4 mL, 1.0 g, 10.1 mmol) in anhydrous CH₂Cl₂ (30 mL), an appropriate acid chloride (10.1 mmol) was added. The mixture was stirred at r.t. overnight and then diluted with H₂O (30 mL). The organic layer was dried over MgSO₄, filtered and evaporated. The product was used without further purification.

### Synthesis of Alkyne 107

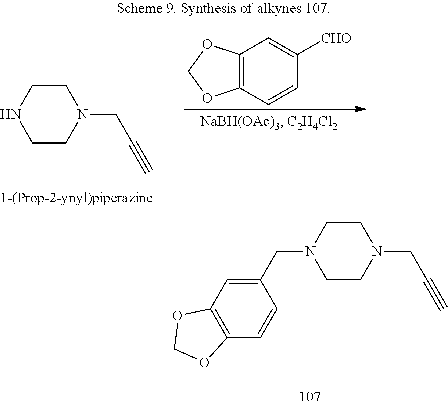

### 1-(Benzo[d][1,3]dioxol-5-ylmethyl)-4-(prop-2-ynyl)piperazine (107)

To a mixture of 1-(prop-2-ynyl)piperazine(129) (1.25 g, 10.1 mmol) and the aldehyde (1.51 g, 10.1 mmol) in anhydrous CH₂Cl₂ (30 mL) and AcOH (12 mg, 0.2 mmol, 2% mol.), NaBH₃CN (0.95 g, 15.1 mmol, 1.5 equiv.) was added. The mixture was stirred at r.t. for one day and then diluted with H₂O (30 mL) and saturated NaHCO₃ (30 mL). The organic layer was dried over MgSO₄, filtered and evaporated. The residue was purified by column chromatography (silica-gel, hexanes:EtOAc, 4:6) to give a yellow solid (1.5 g, 57%).

### Synthesis of Alkynes 109-111

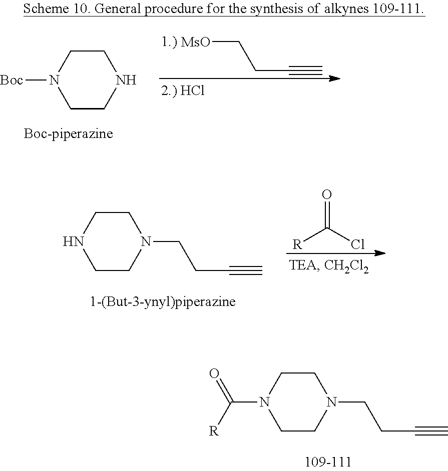

### 1-(But-3-ynyl)piperazine

To a mixture of Boc-piperazine (11.6 g, 62.8 mmol) and but-3-ynyl methanesulfonate 130) (9.3 g, 62.8 mmol) in CH₃CN (130 mL), DIPEA (11.4 mL, 8.9 g, 69 mmol, 1.1 equiv.) was added. The mixture was stirred under reflux overnight and then evaporated. The yellow oil was dissolved in hexanes:EtOAc (1:1), passed through short silica-gel pad and evaporated. The residue was dissolved in HCl_{conc}:H₂O (20 mL:50 mL), stirred at r.t. for 30 min, and then evaporated to give a white solid of 1-(but-3-ynyl)piperazine dihydrochloride (10.5 g, 80%).

General Procedure for the Synthesis of Alkynes 109-111:

To a mixture of 1-(but-3-ynyl)piperazine dihydrochloride (2 g, 9.5 mmol) and TEA (4.4 mL, 3.2 g, 31.3 mmol, 3.3 equiv.) in anhydrous CH₂Cl₂ (20 mL), an appropriate acid chloride (9.5 mmol) was added. The mixture was stirred at r.t. overnight and then diluted with H₂O (30 mL). The organic layer was dried over MgSO₄, filtered and evaporated, and the product was used without further purification.

### Synthesis of Alkynes 112-115

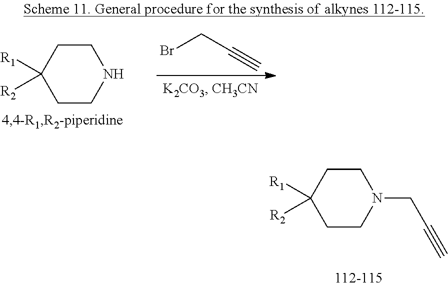

General Procedure:

To a mixture of an appropriate R₁, R₂-substituted piperidine (26 mmol) and DIPEA (4.7 mL, 3.7 g, 28.6 mmol, 1.1 equiv.) in CH₂Cl₂ (100 mL), propargyl bromide (80% solution in toluene, 3.1 mL, 4.2 g, 28.6 mmol, 1.1 equiv.) was added dropwise. The mixture was stirred at r.t. overnight and evaporated. The residue was purified by column chromatography (hexanes:EtOAc).

### Synthesis of Alkynes 130, 132

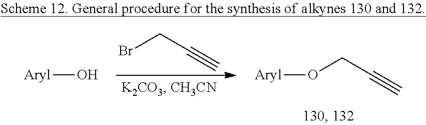

General Procedure:

A mixture of an appropriate phenol (31.7 mmol) and anhydrous K₂CO₃ (4.4 g, 31.7 mmol) in anhydrous CH₃CN (100 mL) was stirred at 50° C. for 15 min, and propargyl bromide (80% solution in toluene, 3.4 mL, 4.7 g, 31.7 mmol) was added. The mixture was stirred at 50° C. for one day, filtered and evaporated. The residue was purified by column chromatography (hexanes:EtOAc or CHCl₃:MeOH).

### Synthesis of Alkyne 116

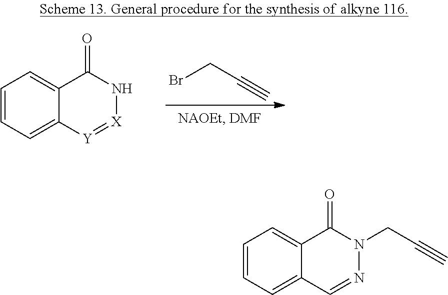

General Procedure:

To a mixture of phthalazone in anhydrous DMF (270 mL), NaOEt (21% w/w solution in EtOH, 51 mL, 137 mmol) was added. After 15 min, propargyl bromide (80% solution in toluene, 14.8 mL, 20.4 g, 137 mmol) was added and the reaction mixture was stirred at r.t. for 2 days. After evaporation, the residue was stirred with hot H₂O (3 L), cooled down and the product was filtered (51%).

### Synthesis of Alkyne 121

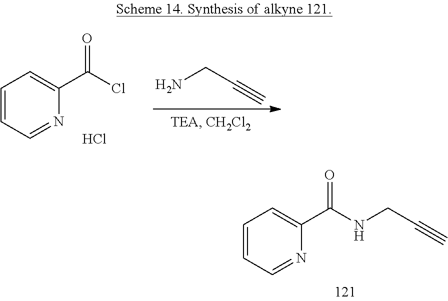

### N-(Prop-2-ynyl)picolinamide (121)

To a cold (0° C.) solution of the acid chloride (5.8 g, 32.6 mmol) in CH₂Cl₂ (50 mL), a mixture of propargyl amine (1.79 g, 32.6 mmol) and TEA (10.0 mL, 7.24 g, 71.7 mmol, 2.2 equiv.) in CH₂Cl₂ (20 mL) was added dropwise. After addition the reaction was stirred at r.t. overnight and washed with H₂O (100 mL). The organic layer was separated, dried over MgSO₄, filtered and evaporated. The residue was dissolved in Et₂O, filtered and the solution was evaporated to the final volume of ˜50 mL. The product was filtered (3 g, 57%).

### Synthesis of Alkyne 123

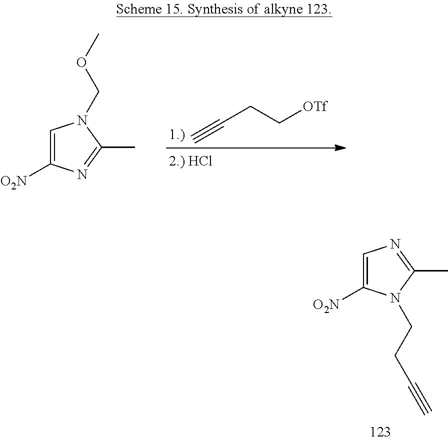

### 1-(But-3-ynyl)-2-methyl-5-nitro-1H-imidazole (123)

1-(Methoxymethyl)-2-methyl-4-nitro-1H-imidazole was obtained using the literature procedure for the similar compound (131). ¹H NMR (400 MHz, CDCl₃) δ=7.78 (s, 1H), 5.23 (s, 2H), 3.35 (s, 3H), 2.48 (s, 3H). ¹³C NMR (100 MHz, CDCl₃) δ=145.4, 119.7, 77.8, 56.6, 13.0. ESI MS (MeOH) for [M+Na]⁺=194.4 Da. To a solution of but-3-ynyl trifluoromethanesulfonate (132) 15.6 g, 77.2 mmol, 1.1 equiv.) in CH₃NO₂ (50 mL) a mixture of 1-(methoxymethyl)-2-methyl-4-nitro-1H-imidazole (12 g, 70.2 mmol) in CH₃NO₂ (50 mL) was added. The mixture was stirred at 70° C. for overnight and then evaporated. The residue was dissolved in 1 M HCl (110 mL) and stirred at 70° C. for 3 h and then cooled to r.t. The reaction mixture was neutralized with 2 M NaOH and extracted using EtOAc. The organic layer was dried over MgSO₄, filtered and evaporated. The residue was purified by column chromatography (silica-gel, Et₂O:MeOH, 95:5) to give 38 (5.2 g, 56%) and then unreacted 1-(methoxymethyl)-2-methyl-4-nitro-1H-imidazole (3.2 g). ¹H NMR (400 MHz, CDCl₃) δ=7.92 (s, 1H), 4.44 (t, J=6.4 Hz, 2H), 2.73-2.69 (m, 2H), 2.54 (s, 3H), 2.01-1.99 (m, 1H). ¹³C NMR (100 MHz, CDCl₃) δ=150.9, 138.2, 133.1, 79.0, 71.7, 44.4, 19.9, 14.6. ESI MS (MeOH) for [M+H]⁺=180.4 Da.

### Synthesis of the Alkyne 124

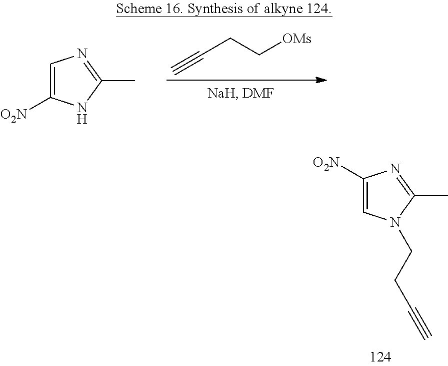

### 1-(But-3-ynyl)-2-methyl-4-nitro-1H-imidazole (124)

To a mixture of NaH (95%, 1.6 g, 66.9 mmol, 1.1 equiv.) in anhydrous DMF (60 mL) a solution of 2-methyl-5-nitro-1H-imidazole (7.7 g, 60.8 mmol) in anhydrous DMF (60 mL) was added dropwise at r.t. After addition, the mixture was stirred at 90° C. for 1 h, then cooled and but-3-ynyl methanesulfonate (130) (9 g, 60.8 mmol) was added. The reaction was stirred at 90° C. overnight and then evaporated. The residue was diluted with brine (200 mL) and H₂O (100 mL) and extracted using EtOAc (2×150 mL). The combined organic layers were dried over MgSO₄, filtered and evaporated. The crude product was dissolved in warm (60° C.) EtOAc (50 mL) and filtered. Et₂O (100 mL) was carefully added (on the top of the EtOAc solution to form biphasic system) and the mixture was kept at r.t. overnight. Crystals of 39 were separated (3.4 g, 31%). ¹H NMR (400 MHz, d₆-DMSO) δ=8.34-8.33 (m, 1H), 4.17-4.12 (m, 2H), 2.95-2.93 (m, 1H), 2.75-2.70 (m, 2H), 2.40-2.39 (m, 3H). ¹³C NMR (100 MHz, d₅-DMSO) δ=145.2, 145.1, 122.0, 80.4, 73.5, 44.7, 19.5, 12.6. ESI MS (MeOH) for [M+Na]⁺=180.3 Da.

### Synthesis of Alkynes 125-126

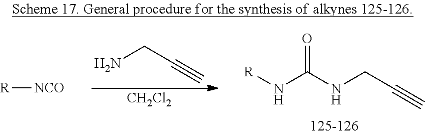

General Procedure:

To a cooled (−25° C.) solution of an appropriate isocyanate (6.2 mmol) in anhydrous CH₂Cl₂ (20 mL), propargylamine (0.39 mL, 340 mg, 6.2 mmol) in anhydrous CH₂Cl₂ (20 mL) was added dropwise. The reaction mixture was stirred at r.t. for 30 min and the product was filtered off, washed with small amount of CH₂Cl₂ and Et₂O.

### Synthesis of Alkynes 127-129

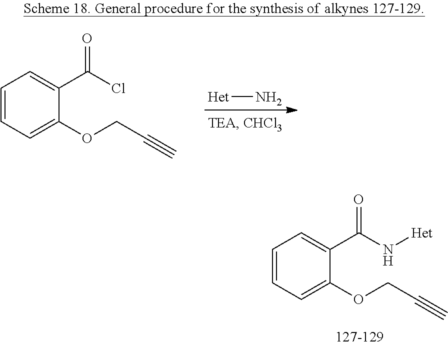

General Procedure:

To a cooled (0° C.) mixture of an appropriate Het-NH₂ (77 mmol) in CHCl₃ (100 mL), the acid chloride (133) (15 g, 77 mmol) in CHCl₃ (50 mL) was added. The reaction was stirred under reflux for one day and then cooled down and washed with H₂O (200 mL). The organic layer was dried over MgSO₄, filtered and evaporated, and the residue was purified by column chromatography (silica-gel, EtOAc).

### Synthesis of Alkyne 133

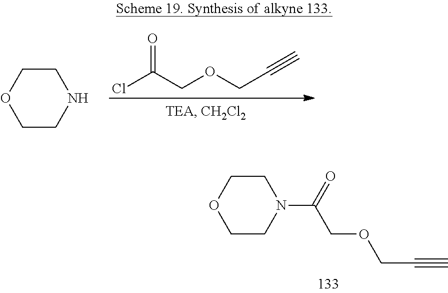

### 1-Morpholino-2-(prop-2-ynyloxy)ethanone (133)

The title compound was obtained using the acid chloride (134) and general procedure as for alkynes 105-106.

### Synthesis of Alkyne 134

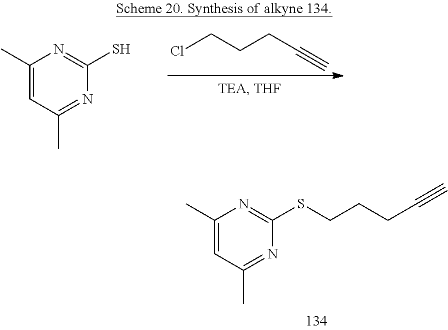

### 4,6-Dimethyl-2-(pent-4-ynylthio)pyrimidine (134)

The title compound was obtained using literature conditions for a similar compound (135).

### Synthesis of Alkyne 136

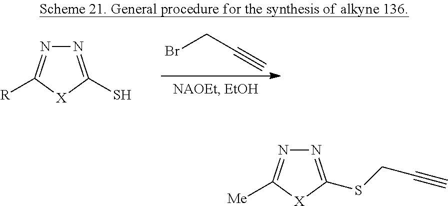

General Procedure:

Alkyne 136 was obtained using literature conditions for a similar compound (136).

### 1.4 List and Synthesis of Alkynes for Triazole Compounds in Test Set

### 1.4.1. List of Alkynes 201-247

The following alkynes were used to synthesize test set compounds. Of these, alkynes 201-223 were newly synthesized, while alkynes 224-247 used in the synthesis

were obtained commercially.

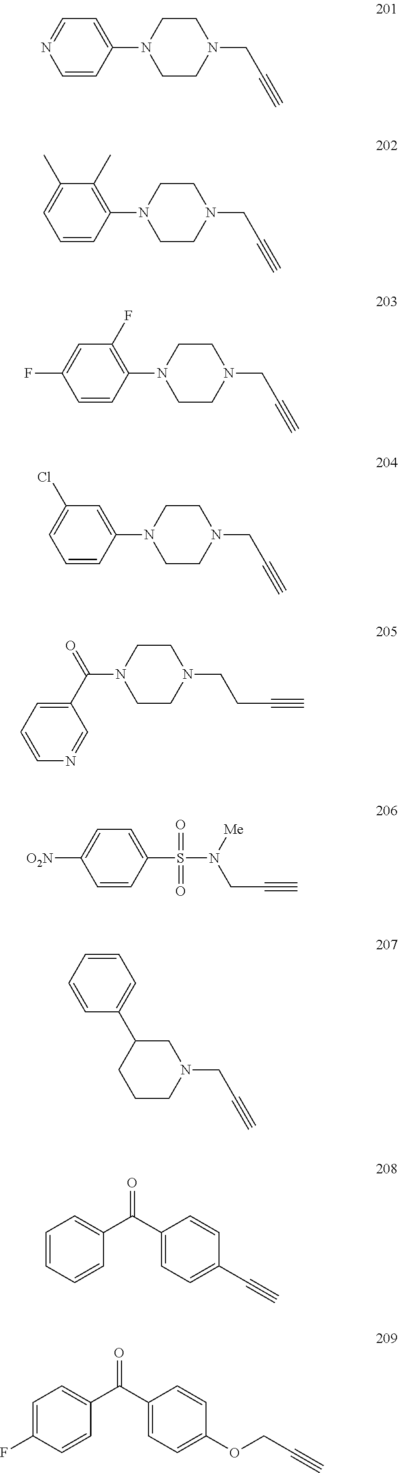

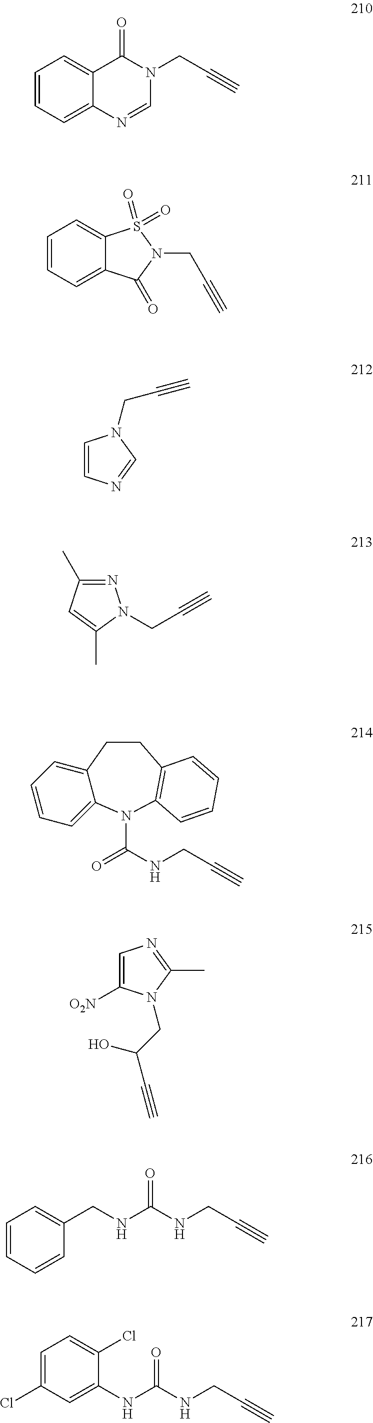

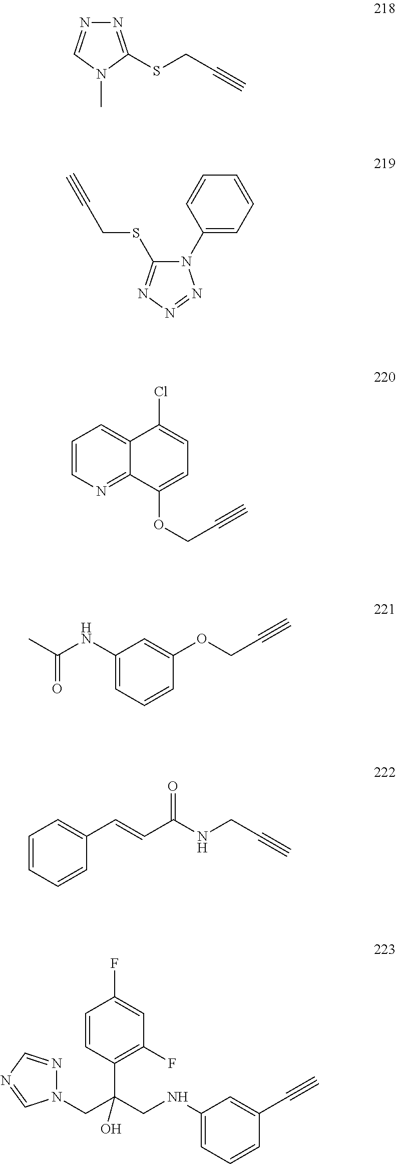

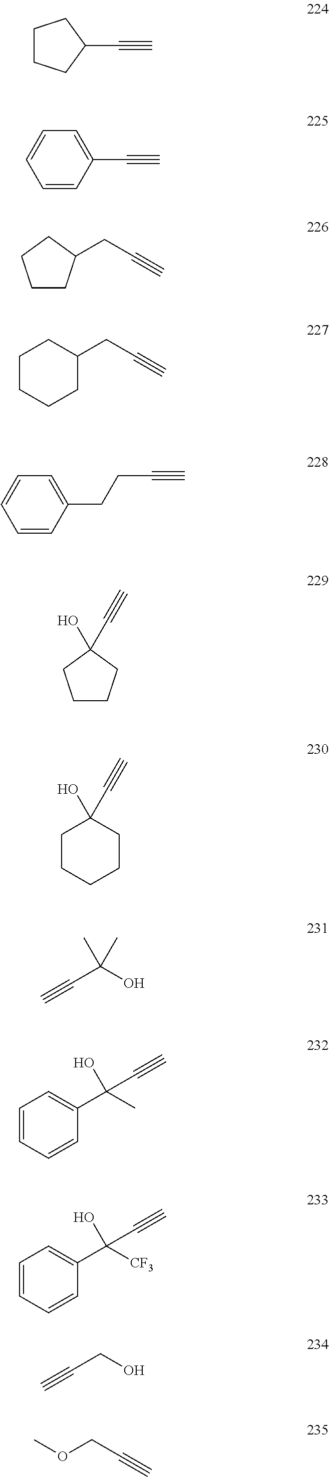

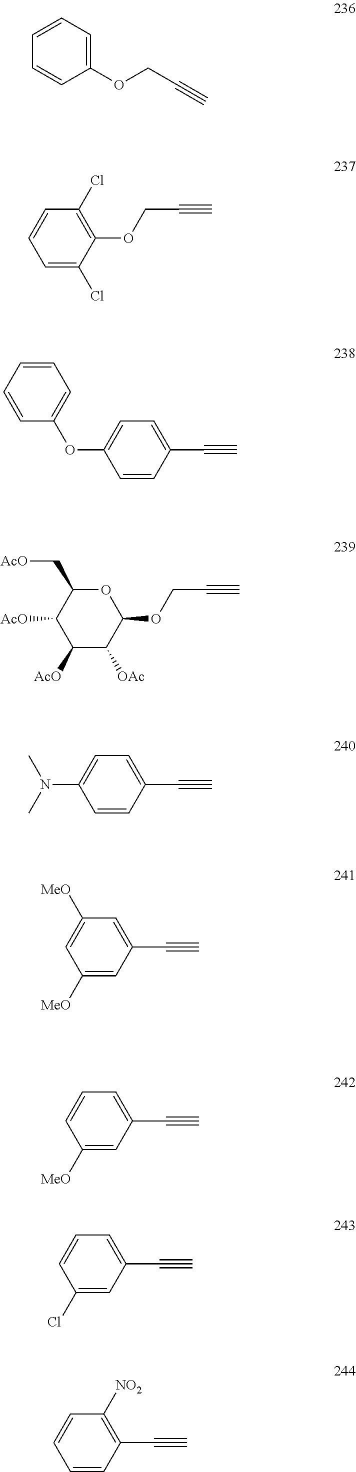

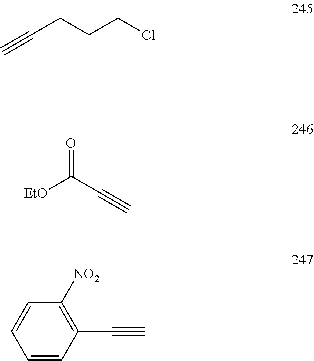

### 1.4.2. Synthesis of Alkynes 201-223

Alkynes 206 (137), 208 (138), 211 (139), 212 (140), 213 (141), 219 (142), and 222 (124) were synthesized according to literature procedures. Alkynes 210 and 220 were described before but without general synthetic procedures (128).

### Synthesis of Alkynes 201-204

The compounds were obtained using Scheme 7 and the general procedure as for alkynes 101-104, 108.

### Synthesis of Alkyne 205

The compound was obtained using Scheme 10 and the general procedure as for alkynes 109-111.

### Synthesis of Alkyne 207

The compound was obtained using Scheme 11 and the general procedure as for alkynes 112-115.

### Synthesis of Alkynes 209, 220-221

The compounds were obtained using Scheme 12 and the general procedure as for alkynes 130, 132.

### Synthesis of Alkyne 210

The compounds were obtained using Scheme 13 (X═CH, Y═N). To 4-hydroxyquinazoline (20 g, 137 mmol) in anhydrous DMF (270 mL), NaOEt (21% w/w solution in EtOH, 51 mL, 137 mmol) was added. After 15 min, propargyl bromide (80% solution in toluene, 14.8 mL, 20.4 g, 137 mmol) was added and the reaction mixture was stirred at r.t. for 2 days. After evaporation, the residue was stirred with hot H₂O (3 L), cooled and the product was filtered (87%).

### Synthesis of Alkyne 214

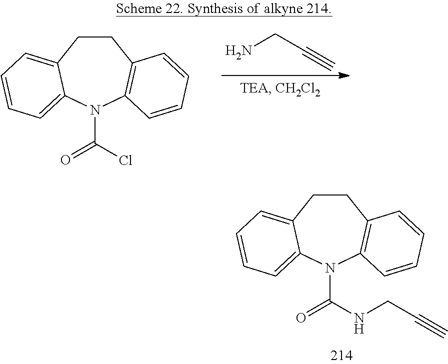

### N-(Prop-2-ynyl)-10,11-dihydro-5H-dibenzo[b,f]azepine-5-carboxamide (214)

To a cold (0° C.) solution of the acid chloride (5 g, 19.4 mmol) in CH₂Cl₂ (100 mL), a mixture of propargyl amine (1.07 g, 19.4 mmol) and TEA (2.7 mL, 1.96 g, 19.4. mmol) in CH₂Cl₂ (50 mL) was added dropwise. After addition the reaction was stirred under reflux for one 1 day, cooled and washed with H₂O (100 mL). The organic layer was separated, dried over MgSO₄, filtered and evaporated. The residue was washed with Et₂O and then hexanes (4.1 g, 76%).

### Synthesis of Alkyne 215

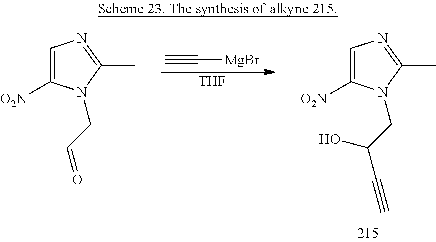

### 1-(2-Methyl-5-nitro-1H-imidazol-1-yl)but-3-yn-2-ol (215)

A mixture of the aldehyde (143) (3.7 g, 21.9 mmol) in anhydrous THF (150 mL) was cooled to −70° C. A solution of ethynylmagnesium bromide (0.5 M in THF, 109 mL, 54.5 mmol, 2.5 equiv.) was added dropwise while maintaining the reaction temperature at −60° C. (the aldehyde was obtained in the hydrate form, and therefore an excess of ethynylmagnesium bromide was used). After addition, the reaction was stirred at −60° C. for 1.5 h and then slowly warmed up to r.t. and stirred for 2 h. The reaction was quenched with brine+H₂O (100+50 mL), filtered through Cclite and washed with EtOAc. The filtrate was transferred to separatory funnel and the organic layer was collected. The aqueous layer was extracted using EtOAc (100 mL). The combined organic fractions were dried over MgSO₄, filtered and evaporated. The residue was purified by column chromatography (silica-gel, EtOAc) (2.7 g, 63%). ¹H NMR (400 MHz, CDCl₃) δ=7.76 (s, 1H), 4.70-4.66 (m, 1H), 4.57-4.52 (m, 1H), 4.41-4.36 (m, 1H), 2.53 (d, J=2.0 Hz, 1H), 2.47 (s, 3H). ¹³C NMR (100 MHz, CDCl₃) δ=151.8, 138.2, 131.8, 81.2, 75.0, 60.3, 50.9, 14.4. ESI MS (MeOH) for [M+H]⁺=196.2 Da.

### Synthesis of Alkynes 216-217

The compounds were obtained using Scheme 19 and the general procedure as for alkynes 125-126.

### Synthesis of Alkyne 218

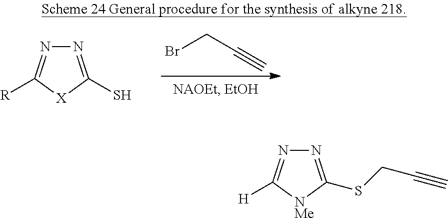

General Procedure:

Alkyne 218 was obtained using literature conditions for a similar compound (136).

### Synthesis of Alkyne 223

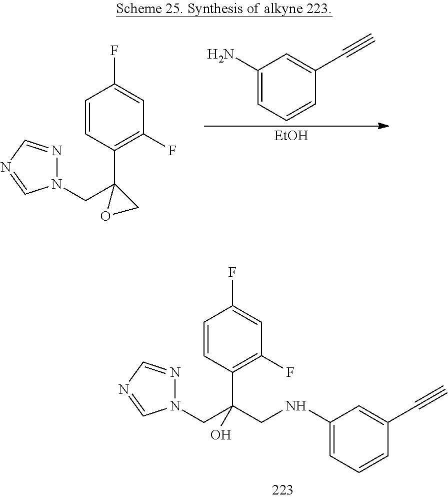

### 2-(2,4-Difluorophenyl)-1-(3-ethynylphenylamino)-3-(1H-1,2,4-triazol-1-yl)propan-2-ol (223)

The title compound was obtained using the literature conditions for a similar compound (144).

### 1.5. “Click Chemistry” Reaction

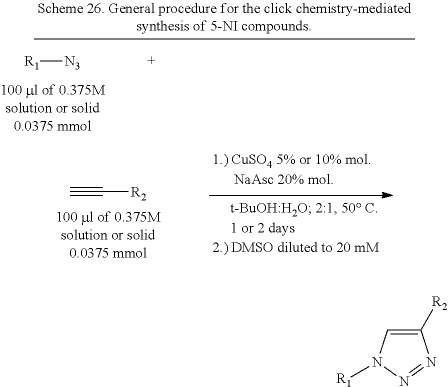

General Procedure:

A 4 mL vial containing a stirring bar was charged with the azide (100 μL of 0.375M) and alkyne (100 μL of 0.375M). Solutions were made in t-BuOH: H₂O; 2:1. In case the azide was not-soluble in the solvent system, solid substrate was added followed by 100 μl of t-BuOH: H₂O; 2:1. If the alkyne was not-soluble in the solvent system, solid substrate was added followed by 100 μl of t-BuOH: H₂O; 2:1. Subsequently, sodium ascorbate (156 μL of 0.048 M) and CuSO₄ (19 μL or 38 μL of 0.1 M in H₂O) were added, giving concentrations of 100 mM each for azide and alkyne, 20 mM sodium ascorbate, 5 or 10 mM CuSO₄, 63.3% t-BuOH, and 36.7% H₂O. The reaction was carried out at 50° C. for 1-2 days. If alkynes reacted slower, the reaction was carried out for 2 days at 50° C. using twice the amount of CuSO₄ (38 μl of 0.1 M in H₂O). The reaction mixture was diluted with DMSO (1.5 mL) and used directly in the biological assays. Final concentrations in reaction mixtures: 20 mM 1,2,3-triazole product, 4 mM sodium ascorbate, 1 or 2 mM CuSO₄, 80% DMSO, 12.7% t-BuOH, and 7.3% H₂O.

### 1.6. Synthesis of Compounds for In Vivo Testing

General Procedure:

To a mixture of azide (2 mmol) and alkyne (2 mmol) in t-BuOH:H₂O (2:1, 10 mL), sodium ascorbate (158 mg, 0.8 mmol, 40% mol.) was added and the mixture was stirred at r.t. for 5 min A 1 M solution of CuSO₄ (200 μL, 0.2 mmol, 10% mol.) was added and the reaction was carried out at 60° C. for 1-3 days. After evaporation, the residue was purified by column chromatography (silica-gel, EtOAc or CHCl₃:MeOH, 9:1 or EtOAc:MeOH, 9:1).

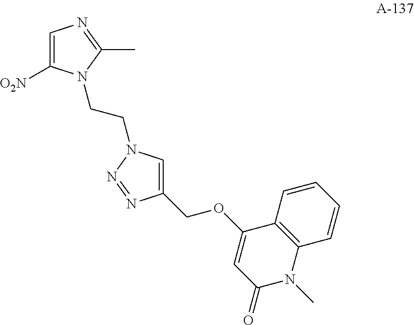 [SMILES: Cc1ncc([N+](=O)[O-])n1CCn1cc(COc2cc(=O)n(C)c3ccccc23)nn1]

### 1-Methyl-4-((1-(2-(2-methyl-5-nitro-1H-imidazol-1-yl)ethyl)-1H-1,2,3-triazol-4-yl)methoxy)quinolin-2(1H)-one (A-137)

The title compound was prepared according to the general procedure (1 day and purified by column chromatography (silica-gel, CHCl₃:MeOH, 9:1) to give a white solid (93%), m.p. 212-213° C. ¹H NMR (500 MHz, d₆-DMSO) δ=8.22 (s, 1H), 8.03 (s, 1H), 7.79 (d, J=7.9 Hz, 1H), 7.64 (t, J=7.8 Hz, 1H), 7.50 (d, J=8.6 Hz, 1H), 7.24 (t, J=7.5 Hz, 1H) 6.23 (s, 1H), 5.30 (s, 2H), 4.89 (t, J=5.3 Hz, 2H), 4.74 (t, J=5.2 Hz, 2H), 3.56 (s, 3H), 1.85 (s, 3H). ¹³C NMR (125 MHz, d₆-DMSO) δ=162.2, 160.3, 151.2, 142.0, 139.5, 138.4, 133.2, 131.5, 125.8, 122.6, 121.5, 115.4, 114.6, 97.5, 61.7, 48.9, 46.2, 28.6, 12.8. HRMS calcd for C₁₉H₂₀N₇O₄ m/z 410.1571, meas 410.1571. IR (cm⁻¹): 3130 (m), 1639 (s), 1459 (s), 1236 (a), 763 (s).

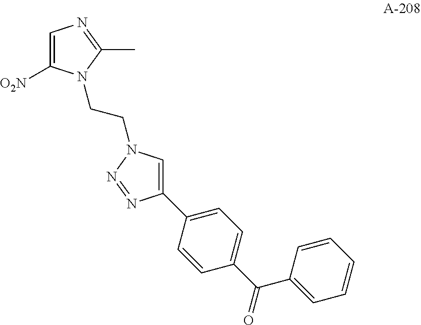 [SMILES: Cc1ncc([N+](=O)[O-])n1CCn1cc(-c2ccc(C(=O)c3ccccc3)cc2)nn1]

### (4-(1-(2-(2-Methyl-5-nitro-1H-imidazol-1-yl)ethyl)-1H-1,2,3-triazol-4-yl)phenyl)(phenyl)methanone (A-208)

The title compound was prepared according to the general procedure (2 days) and purified by column chromatography (silica-gel, EtOAc) to give a white solid (75%), imp. 174-176° C. NMR (500 MHz, d₆-DMSO) δ=8.63 (s, 1H), 8.07 (s, 1H), 7.96 (d, J=8.0 Hz, 2H), 7.83 (d, J=8.0 Hz, 2H), 7.77 (d, J=8.0 Hz, 2H), 7.69 (t, J=7.4 Hz, 1H), 7.58 (t, J=7.5 Hz, 2H), 4.92 (t, J=5.3 Hz, 2H), 4.79 (t, J=5.3 Hz, 2H), 1.95 (s, 3H). ¹³C NMR (125 MHz, d₆-DMSO) δ=195.1, 151.2, 145.6, 138.4, 137.1, 136.2, 134.3, 133.3, 132.6, 130.6, 129.5, 128.6, 125.0, 123.5, 49.0, 46.0, 12.9. HRMS calcd for C₂₁H₁₉N₆O₃ m/z 403.1513, meas 403.1515. IR (cm⁻¹): 3120 (m), 2925 (m), 2852 (m), 1661 (s), 1611 (m), 1452 (s), 1358 (s), 1257 (s), 1200 (s), 1147 (s), 823 (s), 700 (s).

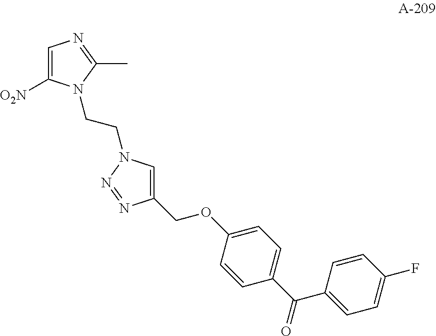 [SMILES: Cc1ncc([N+](=O)[O-])n1CCn1cc(COc2ccc(C(=O)c3ccc(F)cc3)cc2)nn1]

### (4-Fluorophenyl)(4-((1-(2-(2-methyl-5-nitro-1H-imidazol-1-yl)ethyl)-1H-1,2,3-triazol-4-yl)methoxy)phenyl)methanone (A-209)

The title compound was prepared according to the general procedure (2 days) and purified by column chromatography (silica-gel, EtOAc) to give a white solid (78%), m.p. 146-148° C. ¹H NMR (500 MHz, d₆-DMSO) δ=8.16 (s, 1H), 8.04 (s, 1H), 7.79 (dd, J=8.5, 5.6 Hz, 2H), 7.73 (d, J=8.8 Hz, 2H), 7.38 (t, J=8.9 Hz, 2H), 7.16 (d, J=8.6 Hz, 2H), 5.27 (s, 2H), 4.86 (t, J=5.3 Hz, 2H), 4.72 (t, J=5.4 Hz, 2H), 1.79 (s, 3H). ¹³C NMR (125 MHz, d₆-DMSO) δ=193.1, 164.3 (J=250.5 Hz), 161.5, 151.2, 142.5, 138.3, 134.2 (J=2.9 Hz), 133.2, 132.2 (J=9.0 Hz), 132.0, 129.6, 125.6, 115.5 (J=22.0 Hz), 114.7, 61.1, 48.8, 46.1, 12.7. HRMS calcd for C₂₂H₂₀FN₆O₄ m/z 451.1525, meas 451.1545. IR (cm⁻¹): 3131 (m), 1641 (s), 1601 (s), 1458 (s), 1248 (s), 1148 (s), 730 (s).

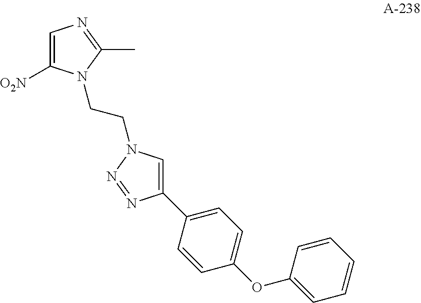 [SMILES: Cc1ncc([N+](=O)[O-])n1CCn1cc(-c2ccc(Oc3ccccc3)cc2)nn1]

### 1-(2-(2-Methyl-5-nitro-1H-imidazol-1-yl)ethyl)-4-(4-phenoxyphenyl)-1H-1,2,3-triazole (A-238)

The title compound was prepared according to the general procedure (2 days) and purified by column chromatography (silica-gel, EtOAc) to give a yellowish solid (90%), imp. 178-179° C. ¹H NMR (500 MHz, d₆-DMSO) δ=8.39 (s, 1H), 8.06 (s, 1H), 7.76 (d, J=8.1 Hz, 2H), 7.41 (t, J=7.7 Hz, 2H), 7.16 (t, J=7.5 Hz, 1H), 7.06 (t, J=7.0 Hz, 4H), 4.88 (t, J=5.5 Hz, 2H), 4.77 (t, J=5.4, 2H), 1.92 (s, 3H). ¹³C NMR (125 MHz, d₆-DMSO) δ=156.5, 156.4, 151.2, 146.1, 138.4, 133.3, 130.1, 126.9, 125.7, 123.6, 121.8, 118.9, 118.8, 48.9, 46.1, 12.9. HRMS calcd for C₂₁H₁₈N₆O₃ m/z 391.1513, meas 391.1501. IR (cm⁻¹): 3148 (m), 3070 (m), 1589 (m), 1558 (w), 1526 (m), 1460 (s), 1365 (s), 1260 (s), 1185 (s), 749 (s).

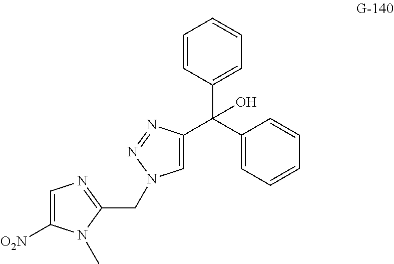 [SMILES: Cn1c([N+](=O)[O-])cnc1Cn1cc(C(O)(c2ccccc2)c2ccccc2)nn1]

### (1((1-Methyl-5-nitro-1H-imidazol-2-371)methyl)-1H-1,2,3-triazol-4-yl)diphenylmethanol (G-140)

The title compound was prepared according to the general procedure (1 day) and purified by column chromatography (silica-gel, EtOAc:MeOH, 9:1) to give a white solid (87%), m.p. 189-191° C. ¹H NMR (500 MHz, d₆-DMSO) δ=8.06 (s, 1H), 7.93 (s, ¹H), 7.37 (d, J=7.9 Hz, 4H), 7.29 (t, J=7.6 Hz, 4H), 7.22 (t, J=7.3 Hz, 2H), 6.60 (s, 1H), 5.90 (s, 2H), 3.96 (s, 3H). ¹³C NMR (125 MHz, d₆-DMSO) δ=154.1, 146.9, 139.5, 131.9, 127.5, 127.0, 126.7, 124.1, 75.6, 45.3, 33.5. HRMS calcd for C₂₀H₁₉N₆O₃ in/z 391.1513, meas 391.1531. IR (cm⁻¹): 3277 (m), 3157 (m), 2984 (w), 2941 (w), 1597 (w), 1527 (w), 1467 (s), 1371 (s), 1180 (s), 1050 (s), 743 (s).

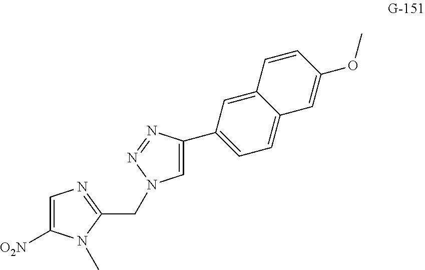 [SMILES: COc1ccc2cc(-c3cn(Cc4ncc([N+](=O)[O-])n4C)nn3)ccc2c1]

### 4-(6-Methoxynaphthalen-2-yl)-1-((1-methyl-5-nitro-1H-imidazol-2-yl)methyl)-1H-1,2,3-triazole (G-151)

The title compound was prepared according to the general procedure (1 day) and purified by column chromatography (silica-gel, CHCl₃:MeOH, 9:1) to give a yellowish solid (58%), m.p. 188-189° C. ¹H NMR (500 MHz, d₆-DMSO) δ=8.66 (s, 1H), 8.34 (s, 1H), 8.10 (s, 1H), 7.96 (dd, J=8.5, 1.2 Hz, 1H), 7.88 (d, J=9.1 Hz, 2H), 7.34 (d, J=2.3 Hz, 1H), 7.19 (dd, J=8.9, 2.4 Hz, 1H), 5.99 (s, 2H), 4.01 (s, 3H), 3.88 (s, 3H). ¹³C NMR (125 MHz, d₆-DMSO) δ=157.5, 146.9, 146.7, 139.6, 133.9, 131.9, 129.5, 128.5, 127.4, 125.6, 124.1, 123.6, 122.0, 119.1, 106.0, 55.2, 45.8, 33.6. HRMS calcd for C₁₈H₁₇N₆O₃ m/z 365.1357, meas 365.1364. IR (cm⁻¹): 3106 (m), 2971 (m), 2843 (m), 1610 (w), 1529 (s), 1468 (s), 1370 (s), 1261 (s), 1218 (s), 1021 (s), 862 (s), 827 (s).

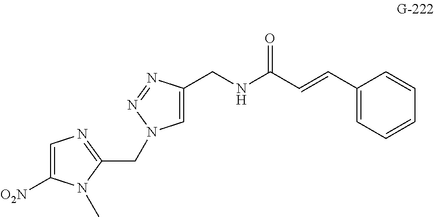 [SMILES: Cn1c([N+](=O)[O-])cnc1Cn1cc(CNC(=O)/C=C/c2ccccc2)nn1]

### N-((1-((1-Methyl-5-nitro-1H-imidazol-2-yl)methyl)-1H-1,2,3-triazol-4-yl)methyl)cinnamamide (G-222)

The title compound was prepared according to the general procedure (2 days) and purified by column chromatography (silica-gel, EtOAc:MeOH, 9:1) to give a white solid (71%), m.p. 160-162° C. ¹H NMR (500 MHz, d₆-DMSO) δ=8.64 (t, 1=5.6 Hz, 1H), 8.05 (s, 2H), 7.55 (d, J=7.7 Hz, 2H), 7.50-7.31 (m, 3H), 6.67 (d, J=15.7 Hz, 1H), 5.89 (s, 2H), 4.44 (d, J=5.8 Hz, 2H), 3.96 (s, 3H), NH broadened and missing in DMSO. ¹³C NMR (125 MHz, d₆-DMSO) δ=165.0, 147.0, 145.0, 139.5, 139.0, 134.8, 131.8, 129.5, 128.9, 127.5, 123.8, 121.8, 45.4, 34.2, 33.5. FIRMS calcd for C₁₇H₁₈N₇O₃ m/z 368.1466, meas 368.1455. IR (cm⁻¹): 3231 (m), 3140 (m), 3046 (m), 2961 (m), 1650 (s), 1535 (s), 1445 (s), 1372 (s), 1052 (s), 825 (s).

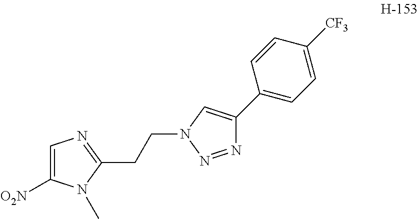 [SMILES: Cn1c([N+](=O)[O-])cnc1CCn1cc(-c2ccc(C(F)(F)F)cc2)nn1]

### 1-(2-(1-methyl-5-nitro-1H-imidazol-2-yl)ethyl)-4-(4-(trifluoromethyl)phenyl)-1H-1,2,3-triazole (H-153)

The title compound was prepared according to the general procedure (1 day) and purified by column chromatography (silica-gel, EtOAc) to give a gray solid (70%), m.p. 137-141° C. ¹H NMR (500 MHz, d₆-DMSO) δ=8.76 (s, 1H), 8.04 (d, J=7.2 Hz, 3H), 7.81 (d, J=8.3, 2H), 4.88 (t, J=6.8 Hz, 2H), 3.83 (s, 3H), 3.46 (t, J=6.9 Hz, 2H). ¹³C NMR (125 MHz, d₆-DMSO) δ=151.1, 145.7, 140.0, 135.5, 133.0, 129.0 (J=3.9 Hz), 126.8 (J=3.8 Hz), 126.5, 125.1 (J=271.9 Hz), 124.0, 47.5, 33.8, 28.3. HRMS calcd for C₁₅H₁₄F₃N₆O₂ m/z 367.1125, meas 367.1131. IR (cm⁻¹): 3135 (m), 1621 (m), 1527 (m), 1465 (m), 1326 (s), 1264 (s), 1185 (s), 1159 (s), 1115 (s), 1064 (s), 812 (s).

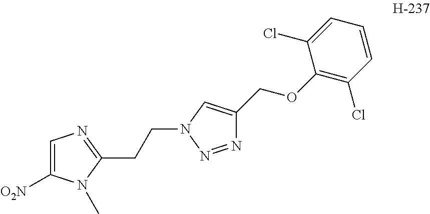 [SMILES: Cn1c([N+](=O)[O-])cnc1CCn1cc(COc2c(Cl)cccc2Cl)nn1]

### 4-((2,6-Dichlorophenoxy)methyl)-1-(2-(1-methyl-5-nitro-1H-imidazol-2-yl)ethyl)-1H-1,2,3-triazole (11-237)

The title compound was prepared according to the general procedure (1 day) and purified by column chromatography (silica-gel, CHCl₃:MeOH, 9:1) to give a white solid (82%), m.p. 125-126° C. ¹H NMR (500 MHz, d₆-DMSO) δ=8.30 (s, 1H), 8.04 (s, 1H), 7.48 (d, J=8.0 Hz, 2H), 7.19 (t, J=8.4 Hz, 1H), 5.20 (s, 2H), 4.83 (t, J=7.0 Hz, 2H), 3.79 (s, 3H), 3.42 (t, J=7.0 Hz, 2H). ¹³C NMR (125 MHz, d₆-DMSO) δ=150.3, 150.1, 141.9, 139.1, 132.1, 129.3, 128.8, 126.3, 125.4, 66.0, 46.5, 32.9, 27.5. HRMS calcd for C₁₅H₁₄Cl₂N₆O₃ m/z 397.0577, meas 397.0560. IR (cm⁻¹): 3135 (m), 3080 (m), 2952 (m), 1566 (w), 1528 (s), 1465 (s), 1446 (s), 1438 (s), 1373 (s), 1186 (s), 968 (s).

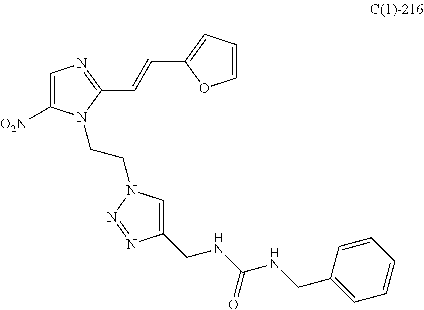 [SMILES: O=C(NCc1ccccc1)NCc1cn(CCn2c([N+](=O)[O-])cnc2/C=C/c2ccco2)nn1]

### (E)-1-Benzyl-3-((1-(2-(2-(2-(furan-2-yl)vinyl)-5-nitro-1H-imidazol-1-yl)ethyl)-1H-1,2,3-triazol-4-yl)methyl)urea (C(1)-216)

The title compound was prepared according to the general procedure (1 day) and purified by column chromatography (silica-gel, CHCl₃:MeOH, 9:1) to give an orange solid (75%), m.p. 153-156° C. ¹H NMR (500 MHz, d₆-DMSO) δ=8.22 (s, 1H), 7.88 (s, 1H), 7.82 (s, 1H), 7.40 (d, J=15.4 Hz, 1H), 7.30 (t, J=7.6 Hz, 2H), 7.25-7.19 (m, 3H), 6.82 (d, J=3.4 Hz, 1H), 6.62 (dd, J=3.4, 1.7 Hz, 1H), 6.39 (t, J=5.9 Hz, 1H), 6.35 (d, J=15.4 Hz, 1H), 6.26 (t, J=5.7 Hz, 1H), 4.91 (t, J=5.6 Hz, 2H), 4.79 (t, J=5.3 Hz, 2H), 4.19 (d, J=5.8 Hz, 2H), 4.05 (d, J=5.7 Hz, 2H). ¹³C NMR (125 MHz, d₆-DMSO) δ=157.7, 151.3, 150.0, 146.2, 145.0, 140.7, 138.5, 135.1, 128.2, 127.0, 126.5, 125.2, 123.3, 113.7, 112.7, 109.3, 48.9, 45.2, 42.9, 34.8. HRMS calcd for C₂₂H₂₃N₈O₄ m/z 463.1837, meas 463.1858. IR (cm⁻¹): 3091 (m), 3029 (m), 1629 (m), 1367 (s), 1200 (s), 741 (s).

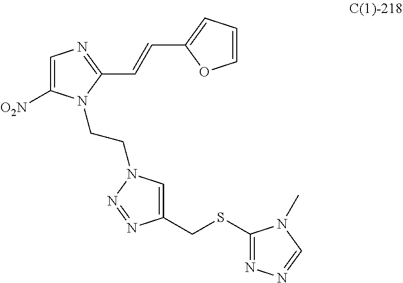 [SMILES: Cn1cnnc1SCc1cn(CCn2c([N+](=O)[O-])cnc2/C=C/c2ccco2)nn1]

### (E)-1-(2-(2-(2-(Furan-2-yl)vinyl)-5-nitro-1H-imidazol-1-yl)ethyl)-4-((4-methyl-4H-1,2,4-triazol-3-ylthio)methyl)-1H-1,2,3-triazole (C(1)-218)

The title compound was prepared according to the general procedure (1 day) and purified by column chromatography (silica-gel, CHCl₃:MeOH, 9:1) to give an orange solid (74%), m.p. 184-186° C. ¹H NMR (500 MHz, d₆-DMSO) δ=8.52 (s, 1H), 8.19 (s, 1H), 7.94 (s, 1H), 7.81 (s, 1H), 7.37 (d, J=15.5 Hz, 1H), 6.81 (d, J=3.3 Hz, 1H), 6.62 (dd, J=3.3, 1.7 Hz, 1H), 6.31 (d, J=15.6 Hz, 1H), 4.89 (t, J=5.3 Hz, 2H), 4.79 (t, J=5.3 Hz, 2H), 4.29 (s, 2H), 3.42 (s, 3H). ¹³C NMR (125 MHz, d₆-DMSO) δ=151.4, 150.0, 148.5, 146.1, 144.9, 142.8, 138.4, 135.1, 125.2, 124.4, 113.7, 112.7, 109.1, 49.0, 45.2, 30.6, 27.2. HRMS calcd for C₁₇H₁₈N₉O₃S m/z 428.1248, meas 428.1264. IR (cm⁻¹): 3091 (m), 3028 (m), 1629 (m), 1420 (s), 1367 (s), 1256 (s), 1190 (s), 931 (s), 741 (s).

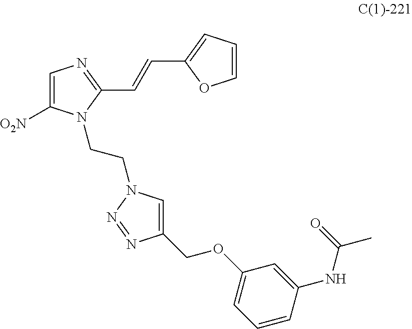 [SMILES: CC(=O)Nc1cccc(OCc2cn(CCn3c([N+](=O)[O-])cnc3/C=C/c3ccco3)nn2)c1]

### (E)-N-(3-((1-(2-(2-(2-(Furan-2-yl)vinyl)-5-nitro-1H-imidazol-1-yl)ethyl)-1H-1,2,3-triazol-4-yl)methoxy)phenyl)acetamide (C(1)-221)

The title compound was prepared according to the general procedure (1 day) and purified by column chromatography (silica-gel, CHCl₃:MeOH, 9:1) to give a yellow solid (77%), m.p. 151-153° C. ¹H NMR (500 MHz, d₆-DMSO) δ=9.89 (s, 1H), 8.22 (s, 1H), 8.14 (s, 1H), 7.80 (s, 1H), 7.37 (d, J=15.4 Hz, 1H), 7.29 (s, 1H), 7.16 (t, 1=8.1 Hz, 1H), 7.08 (d, J=8.0 Hz, 1H), 6.81 (d, J=3.3 Hz, 1H), 6.62-6.57 (m, 2H), 6.35 (d, J=15.2 Hz, 1H), 4.94 (t, J=5.3 Hz, 2H), 4.85 (s, 2H), 4.84 (t, J=5.2 Hz, 2H), 2.03 (s, 3H). ¹³C NMR (125 MHz, d₆-DMSO) δ=158.1, 151.3, 150.1, 144.9, 143.0, 140.4, 138.4, 135.1, 129.4, 125.2, 125.1, 113.7, 112.7, 111.7, 109.2, 108.7, 105.8, 99.1, 60.8, 49.1, 45.2, 24.0. HRMS calcd for C₂₂H₂₂N₇O₅ m/z 464.1677, meas 464.1680. IR (cm⁻¹): 3587 (m), 3302 (m), 3242 (m), 3109 (m), 1666 (s), 1600 (s), 1371 (s), 1260 (s), 1193 (s), 723 (s).

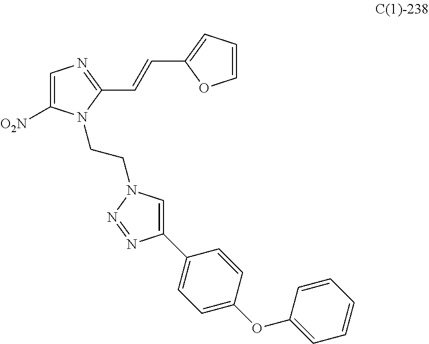 [SMILES: O=[N+]([O-])c1cnc(/C=C/c2ccco2)n1CCn1cc(-c2ccc(Oc3ccccc3)cc2)nn1]

### (E)-1-(2-(2-(2-(Furan-2-yl)vinyl)-5-nitro-1H-imidazol-1-yl)ethyl)-4-(4-phenoxyphenyl)-1H-1,2,3-triazole (C(1)-238)

The title compound was prepared according to the general procedure (3 days) and purified by column chromatography (silica-gel, CHCl₃:MeOH, 9:1) to give a yellow solid (75%), m.p. 221-223° C. NMR (500 MHz, d₆-DMSO) δ=8.45 (s, 1H), 8.23 (s, 1H), 7.69 (s, 1H), 7.60 (d, J=8.2 Hz, 2H), 7.41 (t, J=7.6 Hz, 2H), 7.32 (d, J=15.4 Hz, 1H), 7.16 (t, J=7.4 Hz, 1H), 7.02 (d, J=8.0 Hz, 2H), 6.96 (d, J=8.0 Hz, 2H), 3.32 (d, J=3.2 Hz, 1H), 6.53 (d, J=1.6 Hz, 1H), 6.36 (d, J=15.4 Hz, 1H), 4.98 (t, J=5.0 Hz, 2H), 4.67 (t, J=5.0 Hz, 2H). ¹³C NMR (125 MHz, d₆-DMSO) δ=156.5, 156.2, 151.2, 150.1, 146.2, 144.8, 138.5, 135.3, 130.1, 126.8, 125.9, 125.2, 123.5, 121.8, 118.9, 118.6, 113.7, 112.5, 109.0, 49.3, 45.3. HRMS calcd for C₂₅H₂₁N₆O₄ m/z 469.1619, meas 469.1628. IR (cm⁻¹): 3119 (m), 3096 (m), 1617 (m), 1522 (s), 1487 (s), 1447 (s), 1400 (s), 1294 (s), 1196 (s), 745 (s).

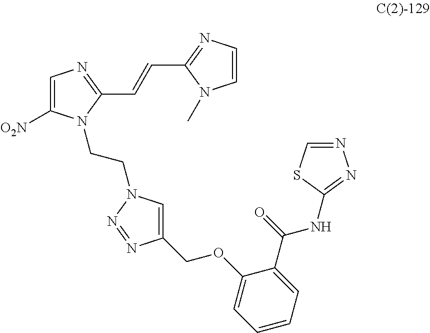 [SMILES: Cn1ccnc1/C=C/c1ncc([N+](=O)[O-])n1CCn1cc(COc2ccccc2C(=O)Nc2nncs2)nn1]

### (E)-2-((1-(2-(2-(2-(1-Methyl-1H-imidazol-2-yl)vinyl)-5-nitro-1H-imidazol-1-yl)ethyl)-1H-1,2,3-triazol-4-yl)methoxy)-N-(1,3,4-thiadiazol-2-yl)benzamide (C(2)-129)

The title compound was prepared according to the general procedure (1 day) and purified by column chromatography (silica-gel, CHCl₃:MeOH, 9:1) to give a yellow solid (86%), m.p. 214-216° C. ¹H NMR (500 MHz, d₆-DMSO) δ=9.24 (s, 1H), 8.25 (s, 1H), 8.23 (s, 1H), 7.80 (d, J=7.8 Hz, 1H), 7.58 (d, J=7.8 Hz, 1H), 7.41 (d, J=15.0 Hz, 1H), 7.25 (d, J=8.4 Hz, 1H), 7.12-7.21 (m, 2H), 6.91 (d, J=15.1 Hz, 1H), 6.41 (s, 1H), 5.06 (s, 2H), 5.02 (s, 2H), 4.83 (s, 2H), 3.68 (s, 3H), missing NH. ¹³C NMR (125 MHz, d₆-DMSO) δ=164.2, 156.0, 150.1, 149.1, 143.8, 143.2, 138.5, 135.1, 133.8, 130.6, 128.5, 124.2, 123.9, 122.8, 121.8, 121.3, 113.5, 113.2, 63.0, 49.5, 48.6, 45.5, 32.6. HRMS calcd for C₂₃H₂₁N₁₁O₄S m/z 548.1571, meas 548.1582. IR (cm⁻¹): 3141 (m), 1664 (m), 1555 (m), 1451 (m), 1438 (m), 1421 (m), 1259 (s), 1195 (s), 1025 (s), 821 (s), 731 (s).

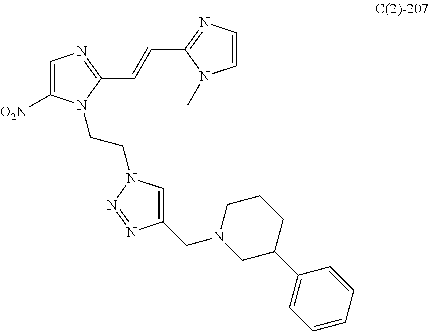 [SMILES: Cn1ccnc1/C=C/c1ncc([N+](=O)[O-])n1CCn1cc(CN2CCCC(c3ccccc3)C2)nn1]

### (E)-1-((1-(2-(2-(2-(1-Methyl-1H-imidazol-2-yl)vinyl)-5-nitro-1H-imidazol-1-yl)ethyl)-1H-1,2,3-triazol-4-yl)methyl)-3-phenylpiperidine (C(2)-207)

The title compound was prepared according to the general procedure (1 day) and purified by column chromatography (silica-gel, CHCl₃:MeOH, 9:1) to give a yellow solid (69%), m.p. broadened decomposition. ¹H NMR (500 MHz, d₆-DMSO) δ=8.21 (s, 1H), 7.88 (s, 1H), 7.47 (d, J=15.1 Hz, 1H), 7.31-7.24 (m, 3H), 7.22-7.14 (m, 3H), 7.05 (d, J=7.0 Hz, 1H), 6.79 (d, J=15.1 Hz, 1H), 4.94 (t, J=4.8 Hz, 2H), 4.80 (t, J=5.0 Hz, 2H), 3.71 (s, 3H), 3.45-3.37 (m, 2H), 2.74 (d, J=10.3 Hz, 1H), 2.69-2.59 (m, 2H), 1.90 (t, J=10.8 Hz, 1H), 1.76 (t, J=10.9 Hz, 1H), 1.71 (d, J=12.3 Hz, 1H), 1.56-1.38 (m, 2H), 1.26 (ddd, J=12.3, 12.3, 4.0 Hz, 1H). ¹³C NMR (125 MHz, d₆-DMSO) δ=149.8, 144.5, 143.3, 143.2, 138.6, 135.0, 129.3, 128.2, 127.1, 126.1, 124.4, 124.2, 123.8, 112.6, 59.9, 52.6, 52.0, 48.9, 45.3, 42.2, 32.6, 30.9, 25.1. HRMS calcd for C₂₅H₃₀N₉O₂ m/z 488.2517, meas 488.2506. IR (cm⁻¹): 3131 (m), 2929 (s), 1631 (m), 1519 (m), 1450 (s), 1400 (s), 1260 (s), 731 (s).

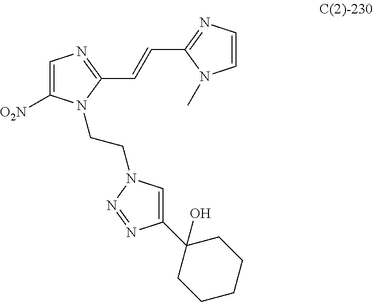 [SMILES: Cn1ccnc1/C=C/c1ncc([N+](=O)[O-])n1CCn1cc(C2(O)CCCCC2)nn1]

### (E)-1-(1-(2-(2-(2-(1-Methyl-1H-imidazol-2-yl)vinyl)-5-nitro-1H-imidazol-1-yl)ethyl)-1H-1,2,3-triazol-4-yl)cyclohexanol (C(2)-230)

The title compound was prepared according to the general procedure (1 day) and purified by column chromatography (silica-gel, CHCl₃:MeOH, 9:1) to give a yellow solid (63%), m.p. 229-230° C. ¹H NMR (500 MHz, d₆-DMSO) δ=8.24 (s, 1H), 7.87 (s, 1H), 7.47 (d, J=15.1 Hz, 1H), 7.28 (s, 1H), 7.04 (s, 1H), 6.64 (d, J=15.0 Hz, 1H), 4.91 (t, J=5.0 Hz, 2H), 4.82-4.77 (m, 3H), 3.71 (s, 3H), 1.67-1.35 (m, 7H), 1.31-1.18 (m, 2H), 1.15-1.00 (m, 1H). ¹³C NMR (500 MHz, d₆-DMSO) δ=156.1, 149.8, 138.6, 135.0, 129.2, 124.2, 124.1, 123.6, 121.6, 112.7, 67.7, 48.8, 45.6, 37.4, 32.6, 25.1, 21.5. HRMS calcd for C₁₉H₂₅N₈O₃ m/z 413.2044, meas 413.2041. IR (cm⁻¹): 3240 (m), 3119 (m), 3098 (m), 2924 (m), 2852 (m), 1635 (m), 1518 (m), 1462 (s), 1369 (s), 1264 (s), 1194 (s), 1178 (s), 979 (s), 740 (s).

### 2. Biological Methods and Data Analysis

### 2.1. Microbial Strains

The following three Mz-sensitive isolates of G. lamblia were used: BRIS/87/HEPU/713 (713) (145), BRIS/83/HEPU/106 (106) (146), and GS/M (ATCC 50580). The Mz-resistant syngeneic lines, 713-M3 and 106-21D10, were derived in the laboratory from isolates 713 and 106, respectively (145, 146). These lines show a stable 10- to 25-fold increase in EC50 for Mz compared to the parental lines (146). For T. vaginalis, we used the two Mz-sensitive clinical isolates, G3 (ATCC PRA-98) and BRIS/92/STDL/F1623 (F1623) (148), and the two Mz-resistant clinical isolates, LA/03/CDC/1 (LAI) (36) and BRIS/92/STDL/B7268 (B7268) (150). Giardia lines were grown in TYI-S-33 medium supplemented with 10% bovine serum and 1 mg/mL bovine bile (Sigma). Trichomonas lines were grown in TYM complete media (151). The H. pylori isolates SS1 and S. Africa R7 (CS22), and the ΔfrxΔdrdxA double-mutant strain of SS1 were described before (152, 153). C. difficile isolate (ATCC 9689) and B. fragilis (ATCC 25285) were obtained from ATCC. The bacterial strains were grown in BHI medium supplemented with 10% bovine fetal serum.

### 2.2. Antimicrobial Assays

Antimicrobial assays were done as described before (147). Briefly, stocks of the test compounds (10 mM in DMSO) were diluted in PBS to 75 μM, and 1:3 serial dilutions were made. Trophozoites were added to the wells in a 96-well plates. Giardia cultures were grown for two days and Trichomonas cultures for one day at 37° C. under anaerobic conditions (AnaeroPack-Anaero system, Remel). For protozoa, cell growth and viability after incubation were determined with an ATP assay by adding BacTiter-Glo microbial cell viability assay reagent (Promega) and measuring ATP-dependent luminescence in a microplate reader. Bacterial cultures were grown for 1 day at 37° C. under anaerobic conditions (AnaeroPack-Anaero system, Remel). Cell growth and viability were determined by optical density measurements at 600 nm. The 50% effective concentration (EC50) was derived from the concentration-response curves using BioAssay software (Cambridge soft). In preliminary studies, we confirmed that EC50 values showed good correlations with minimal inhibitory concentrations (MIC), as determined microscopically with serial dilutions of test compounds and Giardia as a target microbe. However, activity data are reported as EC50 in this study, because the values are more precise and allow better quantitative comparisons.

### 2.3. Cytotoxicity Assay in Mammalian Cells

The human epithelial cell line, HeLa (ATCC CCL-2), was used to determine drug cytotoxicity in human cells (147). Compounds were serially diluted (1:3) and added to HeLa cell cultures in 96-well plates. Cells were grown for 2 days, and viable cell numbers were determined using AlamarBlue reagent (Invitrogen). The 50% cytotoxic concentration (CC50) was derived from the concentration-response curves using BioAssay software (Cambridge soft).

### 2.4. Analysis of Quantitative Structure-Activity Relationship (QSAR)

E-dragon 1.0 from Virtual Computational Chemistry Laboratory was used to calculate 1,666 chemical descriptors for each compound (154). Measured EC50 and chemical descriptor values were used as input attributes for Service Vector Machine calculations and decision tree analyses in WEKA machine learning software (155). To eliminate chemical descriptor redundancy, we used Selected Classifier (Evaluator: CfssubsetEval and search: BestFirst) function with SMO or J48 as classifiers in WEKA. The models were evaluated for significance by Chi-square test. Selected attributes were employed to generate chemical landscapes by Principal Component Analysis (XL-STAT). Graph-R software (version 2.29) was used to generate 3D contour graphs.

### 2.5. Murine Giardia Infection Model

Adult C57BL/6 mice (Jackson Laboratory) were infected orally with 10⁷ trophozoites of G. lamblia GS/M. After 2 days, mice were given five doses of test compound in 0.1% Hypromellose in PBS by oral gavage over a 3-day period. Controls received only 0.1% Hypromellose/PBS. On day 5, the small intestine was removed, opened in 5 ml PBS, and chilled and shaken to release attached trophozoites. Live trophozoites were enumerated in a counting chamber. All animal studies were reviewed and approved by the UCSD Institutional Animal Care and Use Committee.

### 2.6. Assays of Plasma Drug Levels

Mice were given a single 30 mg/kg dose of test compound by oral gavage in 0.1% Hypromellose in PBS. After 2 h, blood samples were taken and centrifuged to collect plasma. A 200 μl volume of plasma was mixed with 1 ml acetone, the mixture was centrifuged, and the supernatant was collected and dried at 50° C. After resuspension in 20 μL complete TYI-SS-33 medium, the plasma concentration of active drug was determined by in vitro antimicrobial assay using G. lamblia 713. As a standard curve, pooled plasma from untreated mice was spiked with different concentrations of test compound, and the samples were processed in parallel with the test samples.

### 2.7. Determination of Water Solubility

Compounds were dissolved in DMSO at 10 mM and serially diluted 1:3 in DMSO. Two μl of each DMSO solution was transferred into 98 μl PBS and shaken for 90 min at room temperature. Crystal formation was assessed by microscopy and the highest compound concentration without crystal formation was used to estimate water solubility.

### 2.8. In Vitro Micronucleus Assays

In vitro micronucleus assays were performed using CHO-K1 cells according to current OECD guidelines (156).

### 2.9. Assessment of In Vivo Toxicity

Adult C57BL/6 mice (Jackson Laboratory) were given five doses of 100 mg/kg of 5-NI compounds orally over a 3-day period. On day 4, blood, small intestine, colon, liver, spleen and kidneys were removed and fixed, and paraffin sections were prepared and stained with hematoxylin and eosin. Standard hematological analysis and determination of plasma levels of albumin, alkaline phosphatase, ALT, AST, bilirubin, creatinine, glucose, and electrolytes were performed in the UCSD Murine Hematology and Coagulation Core Laboratory.

Lengthy table referenced here
US20160244435A1-20160825-T00001
Please refer to the end of the specification for access instructions.

Lengthy table referenced here
US20160244435A1-20160825-T00002
Please refer to the end of the specification for access instructions.

Lengthy table referenced here
US20160244435A1-20160825-T00003
Please refer to the end of the specification for access instructions.

Lengthy table referenced here
US20160244435A1-20160825-T00004
Please refer to the end of the specification for access instructions.

Lengthy table referenced here
US20160244435A1-20160825-T00005
Please refer to the end of the specification for access instructions.

Lengthy table referenced here
US20160244435A1-20160825-T00006
Please refer to the end of the specification for access instructions.

Lengthy table referenced here
US20160244435A1-20160825-T00007
Please refer to the end of the specification for access instructions.

Lengthy table referenced here
US20160244435A1-20160825-T00008
Please refer to the end of the specification for access instructions.

Lengthy table referenced here
US20160244435A1-20160825-T00009
Please refer to the end of the specification for access instructions.

Lengthy table referenced here
US20160244435A1-20160825-T00010
Please refer to the end of the specification for access instructions.

Lengthy table referenced here
US20160244435A1-20160825-T00011
Please refer to the end of the specification for access instructions.

Lengthy table referenced here
US20160244435A1-20160825-T00012
Please refer to the end of the specification for access instructions.

Lengthy table referenced here
US20160244435A1-20160825-T00013
Please refer to the end of the specification for access instructions.

Lengthy table referenced here
US20160244435A1-20160825-T00014
Please refer to the end of the specification for access instructions.

Lengthy table referenced here
US20160244435A1-20160825-T00015
Please refer to the end of the specification for access instructions.

Lengthy table referenced here
US20160244435A1-20160825-T00016
Please refer to the end of the specification for access instructions.

Lengthy table referenced here
US20160244435A1-20160825-T00017
Please refer to the end of the specification for access instructions.

Lengthy table referenced here
US20160244435A1-20160825-T00018
Please refer to the end of the specification for access instructions.

Lengthy table referenced here
US20160244435A1-20160825-T00019
Please refer to the end of the specification for access instructions.

Lengthy table referenced here
US20160244435A1-20160825-T00020
Please refer to the end of the specification for access instructions.

Lengthy table referenced here
US20160244435A1-20160825-T00021
Please refer to the end of the specification for access instructions.

Lengthy table referenced here
US20160244435A1-20160825-T00022
Please refer to the end of the specification for access instructions.

The present disclosure is not to be limited in terms of the particular embodiments described in this application, which are intended as illustrations of various aspects. Many modifications and variations can be made without departing from its spirit and scope, as are apparent to those skilled in the art. Functionally equivalent methods and apparatuses within the scope of the disclosure, in addition to those enumerated herein, are apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

Each of the following references is incorporated herein by reference in its entirety.

### REFERENCES INCORPORATED BY REFERENCE

1. Drawz S M & Bonomo R A (2010) Three decades of beta-lactamase inhibitors. Clin. Microbiol. Rev. 23(1):160-201.
2. Page M G, Dantier C, & Desarbre E (2010) In vitro properties of BAL30072, a new siderophore sulfactam with activity against multiresistant gram-negative bacilli. Antimicrob. Agents Chemother. 54(6):2291-2302.
3. Brook I (2011) Antimicrobial treatment of anaerobic infections. Expert Opin. Pharmacother. 12(11):1691-1707.
4. Uperoft P & Uperoft J A (2001) Drug targets and mechanisms of resistance in the anaerobic protozoa. Clin. Microbiol. Rev. 14(1):150-164.
5. Edwards D I (1993) Nitroimidazole drugs—action and resistance mechanisms. I. Mechanisms of action. J. Antimicrob. Chemother. 31(1):9-20.
6. Valdez C A, et al. (2009) Synthesis and Electrochemistry of 2-Ethenyl and 2-Ethanyl Derivatives of 5-Nitroimidazole and Antimicrobial Activity against Giardia lamblia. J. Med. Chem. 52(13):4038-4053.
7. Lofrnark S, Edlund C, & Nord C E (2010) Metronidazole is still the drug of choice for treatment of anaerobic infections. Clin. Infect. Dis. 50 Suppl 1:S16-23.
8. Secka O, et al. (2013) Antimicrobial susceptibility and resistance patterns among Helicobacter pylori strains from The Gambia, West Africa. Antimicrob. Agents Chemother. 57(3):1231-1237.
9. Krashin J W, et al. (2010) Trichomonas vaginalis prevalence, incidence, risk factors and antibiotic-resistance in an adolescent population. Sex. Transm. Dis. 37(7):440-444.
10. Tejman-Yarden N, et al. (2011) Impaired Parasite Attachment as Fitness Cost of Metronidazole Resistance in Giardia lamblia. Antimicrob. Agents Chemother. 55(10):4643-4651.
11. Dan M, Wang A L, & Wang C C (2000) Inhibition of pyruvate-ferredoxin oxidoreductase gene expression in Giardia lamblia by a virus-mediated hammerhead ribozyme. Mol. Microbiol. 36(2):447-456.
12. Leitsch D, et al. (2011) Pyruvate:ferredoxin oxidoreductase and thioredoxin reductase are involved in 5-nitroimidazole activation while flavin metabolism is linked to 5-nitroimidazole resistance in Giardia lamblia. J. Antimicrob. Chemother. 66(8):1756-1765.
13. Townson S M, Uperoft J A, & Uperoft P (1996) Characterisation and purification of pyruvate:fenedoxin oxidoreductase from Giardia duodenalis. Mol. Biochem. Parasitol. 79(2):183-193.
14. Pal D, et al. (2009) Giardia, Entamoeba, and Trichomonas Enzymes Activate Metronidazole (Nitroreductases) and Inactivate Metronidazole (Nitroimidazole Reductases). Antimicrob. Agents Chemother. 53(2):458-464.
15. Uperoft J A, et al. (2006) 5-Nitroimidazole drugs effective against metronidazole-resistant Trichomonas vaginalis and Giardia duodenalis. Antimicrob. Agents Chemother. 50(1):344-347.
16. Bahia M T, et al. (2012) Fexinidazole: a potential new drug candidate for chagas disease. PLoS Negl. Trop. Dis. 6(11):e1870.
17. Bendesky A, Menendez D, & Ostrosky-Wegman P (2002) Is metronidazole carcinogenic? Mutat. Res. 511(2):133-144.
18. Hein J E & Fokin V V (2010) Copper-catalyzed azide-alkyne cycloaddition (CuAAC) and beyond: new reactivity of copper(i) acetylides. Chem. Soc. Rev. 39(4):1302-1315.
19. Kolb H C, Finn M G, & Sharpless K B (2001) Click Chemistry: Diverse Chemical Function from a Few Good Reactions. Angew. Chem. Int. Ed. Engl. 40(11):2004-2021.
20. Kolb H C & Sharpless K B (2003) The growing impact of click chemistry on drug discovery. Drug Discovery Today 8(24):1128-1137.
21. Pierce K K & Kirkpatrick B D (2009) Update on human infections caused by intestinal protozoa. Curr. Opin. Gastroen. 25(1):12-17.
22. Johnston V J & Mabey D C (2008) Global epidemiology and control of Trichomonas vaginalis. Curr. Opin. Infect. Dis. 21(1):56-64.
23. Sibony M & Jones N L (2012) Recent advances in Helicobacter pylori pathogenesis. Curr. Opin. Gastroen. 28(1):30-35.
24. Lessa F C, Gould C V, & McDonald L C (2012) Current status of Clostridium difficile infection epidemiology. Clin. Infect. Dis. 55 Suppl 2:S65-70.
25. Jeong J Y, et al. (2001) Roles of FrxA and RdxA nitroreductases of Helicobacter pylori in susceptibility and resistance to metronidazole. J. Bacteria 183(17):5155-5162.
26. Gamo F J, et al. (2010) Thousands of chemical starting points for antimalarial lead identification. Nature 465(7296):305-310.
27. Guiguemde W A, et al. (2010) Chemical genetics of Plasmodium falciparum. Nature 465(7296):311-315.
28. Reymond J-L, van Deursen R, Blum L C, & Ruddigkeit L (2010) Chemical space as a source for new drugs. Med. Chem. Comm 1(1):30-38.
29. Ghose A K, Viswanadhan V N, & Wendoloski J J (1999) A knowledge-based approach in designing combinatorial or medicinal chemistry libraries for drug discovery. 1. A qualitative and quantitative characterization of known drug databases. J. Comb. Chem. 1(1):55-68.
30. Lipinski C A, Lombardo F, Dominy B W, & Feeney P J (1997) Experimental and computational approaches to estimate solubility and permeability in drug discovery and development settings. Adv. Drug Deliv. Rev. 23(1-3):3-25.
31. Bickerton G R, Paolini G V, Besnard J, Muresan S, & Hopkins A L (2012) Quantifying the chemical beauty of drugs. Nat. Chem. 4(2):90-98.
32. Aravena C M, et al. (2011) Potent 5-nitrofuran derivatives inhibitors of Trypanosoma cruzi growth: electrochemical, spectroscopic and biological studies. Spectrochim. Acta. A. Mol. Biomol. Spectrosc. 79(2):312-319.
33. Leitsch D, Kolarich D, Wilson I B, Altmann F, & Duchene M (2007) Nitroimidazole action in Entamoeba histolytica: a central role for thioredoxin reductase. PLoS Biol. 5(8):e211.
34. Land K M & Johnson P J (1999) Molecular basis of metronidazole resistance in pathogenic bacteria and protozoa. Drug Resist. Update. 2(5):289-294.
35. Nillius D, Muller J, & Muller N (2011) Nitroreductase (GlNR1) increases susceptibility of Giardia lamblia and Escherichia coli to nitro drugs. J. Antimicrob. Chemother. 66(5):1029-1035.
36. Pumbwe L, Glass D, & Wexler H M (2006) Efflux pump overexpression in multiple-antibiotic-resistant mutants of Bacteroides fragilis. Antimicrob. Agents Chemother. 50(9):3150-3153.
37. Nagle A, et al. (2012) Imidazolopiperazines: lead optimization of the seconJ-generation antimalarial agents. J. Med. Chem. 55(9):4244-4273.
38. Tetko I V, et al. (2005) Virtual computational chemistry laboratory—design and description. J. Comput. Aided. Mol. Des. 19(6):453-463.
39. Hall M, et al. (2009) The WEKA Data Mining Software: An Update. SIGKDD Explorations 11(1).
40. Uperoft, P., and J. A. Uperoft. 2001. Drug targets and mechanisms of resistance in the anaerobic protozoa. Clin Microbiol Rev 14:150-164.
41. Ankarklev, J., J. Jerlstrom-Hultqvist, E. Ringqvist, K. Troell, and S. G. Svard. 2010. Behind the smile: cell biology and disease mechanisms of Giardia species. Nat Rev Microbiol 8:413-422.
42. Fletcher, S. M., D. Stark, J. Harkness, and J. Ellis. 2012. Enteric protozoa in the developed world: a public health perspective. Clin Microbiol Rev 25:420-449.
43. Craun, G. F., R. L. Calderon, and M. F. Craun. 2005. Outbreaks associated with recreational water in the United States. Int J Environ Health Res 15:243-262.
44. Yoder, J. S., J. W. Gargano, R. M. Wallace, and M. J. Beach. 2012. Giardiasis surveillance—United States, 2009-2010. MMWR Surveill Summ 61:13-23.
45. Leclerc, H., L. Schwartzbrod, and E. Dei-Cas. 2002. Microbial agents associated with waterborne diseases. Crit Rev Microbiol 28:371-409.
46. Rendtorff, R. C. 1954. The experimental transmission of human intestinal protozoan parasites. II. Giardia lamblia cysts given in capsules. Am J Hyg 59:209-220.
47. Nash, T. E., D. A. Herrington, G. A. Losonsky, and M. M. Levine. 1987. Experimental human infections with Giardia lamblia. J Infect Dis 156:974-984.
48. Adam, R. D. 2001. Biology of Giardia lamblia. Clin Microbiol Rev 14:447-475.
49. Cotton, J. A., J. K. Beatty, and A. G. Buret. 2011. Host parasite interactions and pathophysiology in Giardia infections. International journal for parasitology 41:925-933.
50. Nash, T. E. 2013. Unraveling how Giardia infections cause disease. The Journal of clinical investigation:1-2.
51. Petri, W. A., Jr. 2000. Protozoan parasites that infect the gastrointestinal tract. Curr Opin Gastroenterol 16:18-23.
52. Morch, K., K. Hanevik, G. Rortveit, K. A. Wensaas, and N. Langeland. 2009. High rate of fatigue and abdominal symptoms 2 years after an outbreak of giardiasis. Trans R Soc Trop Med Hyg 103:530-532.
53. Berkman, D. S., A. G. Lescano, R. H. Gilman, S. L. Lopez, and M. M. Black. 2002. Effects of stunting, diarrhoeal disease, and parasitic infection during infancy on cognition in late childhood: a follow-up study. Lancet 359:564-571.
54. Lofmark, S., C. Edlund, and C. E. Nord. 2010. Metronidazole is still the drug of choice for treatment of anaerobic infections. Clin Infect Dis 50 Suppl 1:S16-23.
55. Gardner, T. B., and D. R. Hill. 2001. Treatment of giardiasis. Clin Microbiol Rev 14:114-128.
56. Jokipii, L., and A. M. Jokipii. 1979. Single-dose metronidazole and tinidazole as therapy for giardiasis: success rates, side effects, and drug absorption and elimination. J Infect Dis 140:984-988.
57. Speelman, P. 1985. Single-dose tinidazole for the treatment of giardiasis. Antimicrob Agents Chemother 27:227-229.
58. Tejman-Yarden, N., M. Millman, T. Lauwaet, B. J. Davids, F. D. Gillin, L. Dunn, J. A. Uperoft, Y. Miyamoto, and L. Eckmann. 2011. Impaired parasite attachment as fitness cost of metronidazole resistance in Giardia lamblia. Antimicrob Agents Chemother 55:4643-4651.
59. Solaymani-Mohammadi, S., J. M. Genkinger, C. A. Loffredo, and S. M. Singer. 2010. A meta-analysis of the effectiveness of albendazole compared with metronidazole as treatments for infections with Giardia duodenalis. PLoS neglected tropical diseases 4:e682.
60. Hanevik, K., K. Morch, G. E. Eide, N. Langeland, and T. Hausken. 2008. Effects of albendazole/metronidazole or tetracycline/folate treatments on persisting symptoms after Giardia infection: a randomized open clinical trial. Scandinavian journal of infectious diseases 40:517-522.
61. Wright, J. M., L. A. Dunn, P. Uperoft, and J. A. Uperoft. 2003. Efficacy of antigiardial drugs. Expert Opin Drug Saf 2:529-541.
62. Tejman-Yarden, N., and L. Eckmann. 2011. New approaches to the treatment of giardiasis. Current opinion in infectious diseases 24:451-456.
63. Tejman-Yarden, N., Y. Miyamoto, D. Leitsch, J. Santini, A. Debnath, J. Gut, J. H. McKerrow, S. L. Reed, and L. Eckmann. 2013. A reprofiled drug, auranofin, is effective against metronidazole-resistant Giardia lamblia. Antimicrob Agents Chemother 57:2029-2035.
64. Brown, D. M., J. A. Uperoft, and P. Uperoft. 1996. A H2O-producing NADH oxidase from the protozoan parasite Giardia duodenalis. Eur J Biochem 241:155-161.
65. Townson, S. M., G. R. Hanson, J. A. Uperoft, and P. Uperoft. 1994. A purified ferredoxin from Giardia duodenalis. Eur J Biochem 220:439-446.
66. Townson, S. M., J. A. Uperoft, and P. Uperoft. 1996. Characterisation and purification of pyruvate:ferredoxin oxidoreductase from Giardia duodenalis. Mol Biochem Parasitol 79:183-193.
67. Edwards, D. I. 1993. Nitroimidazole drugs—action and resistance mechanisms. I. Mechanisms of action. J Antimicrob Chemother 31:9-20.
68. Leitsch, D., A. G. Burgess, L. A. Dunn, K. G. Krauer, K. Tan, M. Duchene, P. Uperoft, L. Eckmann, and J. A. Uperoft. 2011. Pyruvate:ferredoxin oxidoreductase and thioredoxin reductase are involved in 5-nitroimidazole activation while flavin metabolism is linked to 5-nitroimidazole resistance in Giardia lamblia. J Antimicrob Chemother.
69. Pal, D., S. Banerjee, J. Cui, A. Schwartz, S. K. Ghosh, and J. Samuelson. 2009. Giardia, Entamoeba, and Trichomonas enzymes activate metronidazole (nitroreductases) and inactivate metronidazole (nitroimidazole reductases). Antimicrob Agents Chemother 53:458-464.
70. Muller, J., P. Schildknecht, and N. Muller. 2013. Metabolism of nitro drugs metronidazole and nitazoxanide in Giardia lamblia: characterization of a new nitroreductase (GlNR2). J Antimicrob Chemother.
71. Nillius, D., J. Muller, and N. Muller. 2011. Nitroreductase (GlNR1) increases susceptibility of Giardia lamblia and Escherichia coli to nitro drugs. J Antimicrob Chemother 66:1029-1035.
72. Leitsch, D., D. Kolarich, I. B. Wilson, F. Altmann, and M. Duchene. 2007. Nitroimidazole action in Entamoeba histolytica: a central role for thioredoxin reductase. PLoS Biol 5:e211.
73. Dunn, L. A., A. G. Burgess, K. G. Krauer, L. Eckmann, P. Vanelle, M. D. Crozet, F. D. Gillin, P. Uperoft, and J. A. Uperoft. 2010. A new-generation 5-nitroimidazole can induce highly metronidazole-resistant Giardia lamblia in vitro. Int J Antimicrob Agents 36:37-42.
74. Townson, S. M., H. Laqua, P. Uperoft, P. F. Boreham, and J. A. Uperoft. 1992. Induction of metronidazole and furazolidone resistance in Giardia. Trans R Soc Trop Med Hyg 86:521-522.
75. Boreham, P. F., R. E. Phillips, and R. W. Shepherd. 1988. Altered uptake of metronidazole in vitro by stocks of Giardia intestinalis with different drug sensitivities. Trans R Soc Trop Med Hyg 82:104-106.
76. Dan, M., A. L. Wang, and C. C. Wang. 2000. Inhibition of pyruvate-ferredoxin oxidoreductase gene expression in Giardia lamblia by a virus-mediated hammerhead ribozyme. Mol Microbiol 36:447-456.
77. Uperoft, J. A., L. A. Dunn, J. M. Wright, K. Benakli, P. Uperoft, and P. Vanelle. 2006. 5-Nitroimidazole drugs effective against metronidazole-resistant Trichomonas vaginalis and Giardia duodenalis. Antimicrob Agents Chemother 50:344-347.
78. Endimiani, A., F. Perez, and R. A. Bonomo. 2008. Cefepime: a reappraisal in an era of increasing antimicrobial resistance. Expert review of anti-infective therapy 6:805-824.
79. Bercu, T. E., W. A. Petri, and J. W. Behm. 2007. Amebic colitis: new insights into pathogenesis and treatment. Current gastroenterology reports 9:429-433.
80. Mortimer, L., and K. Chadee. 2010. The immunopathogenesis of Entamoeba histolytica. Exp Parasitol 126:366-380.
81. Wassmann, C., A. Hellberg, E. Tannich, and I. Bruchhaus. 1999. Metronidazole resistance in the protozoan parasite Entamoeba histolytica is associated with increased expression of iron-containing superoxide dismutase and peroxircdoxin and decreased expression of ferredoxin 1 and flavin reductase. J Biol Chem 274:26051-26056.
82. Uperoft, J. A., R. W. Campbell, K. Benakli, P. Uperoft, and P. Vanelle. 1999. Efficacy of new 5-nitroimidazoles against metronidazole-susceptible and -resistant Giardia, Trichomonas, and Entamoeba spp. Antimicrob Agents Chemother 43:73-76.
83. Valdez, C. A., J. C. Tripp, Y. Miyamoto, J. Kalisiak, P. Hruz, Y. S. Andersen, S. E. Brown, K. Kangas, L. V. Arzu, B. J. Davids, F. D. Gillin, J. A. Uperoft, P. Uperoft, V. V. Fokin, D. K. Smith, K. B. Sharpless, and L. Eckmann. 2009. Synthesis and electrochemistry of 2-ethenyl and 2-ethanyl derivatives of 5-nitroimidazole and antimicrobial activity against Giardia lamblia. J Med Chem 52:4038-4053.
84. Moraski, G. C., S. G. Franzblau, and M. J. Miller. 2010. Utilization of the Suzuki Coupling to Enhance the Antituberculosis Activity of Aryl Oxazoles. Heterocycles 80:977-988.
85. Kolb, H. C., and K. B. Sharpless. 2003. The growing impact of click chemistry on drug discovery. Drug Discov Today 8:1128-1137.
86. Thompson, R. C., R. M. Hopkins, and W. L. Homan. 2000. Nomenclature and genetic groupings of Giardia infecting mammals. Parasitol Today 16:210-213.
87. Liu, S. M., D. M. Brown, P. O'Donoghue, P. Uperoft, and J. A. Uperoft. 2000. Ferredoxin involvement in metronidazole resistance of Giardia duodenalis. Mol Biochem Parasitol 108:137-140.
88. Lemee, V., I. Zaharia, G. Nevez, M. Rabodonirina, P. Brasseur, J. J. Ballet, and L. Favennec. 2000. Metronidazole and albendazole susceptibility of 11 clinical isolates of Giardia duodenalis from France. J Antimicrob Chemother 46:819-821.
89. Bouhifd, M., G. Bones, J. Casado, S. Coecke, H. Norlen, N. Parissis, R. M. Rodrigues, and M. P. Whelan. 2012. Automation of an in vitro cytotoxicity assay used to estimate starting doses in acute oral systemic toxicity tests. Food and chemical toxicology: an international journal published for the British Industrial Biological Research Association 50:2084-2096.
90. He, C., G. P. Nora, E. L. Schneider, I. D. Kerr, E. Hansell, K. Hirata, D. Gonzalez, M. Sajid, S. E. Boyd, P. Hruz, E. R. Cobo, C. Le, W. T. Liu, L. Eckmann, P. C. Donestein, E. R. Houpt, L. S. Brinen, C. S. Craik, W. R. Roush, J. McKerrow, and S. L. Reed. 2010. A new Entamoeba histolytica cysteine proteinase, EhCP4, is key for invasive amebiasis and a therapeutic target. J Biol Chem 285:18516-18527.
91. Cobo, E. R., C. He, K. Hirata, G. Hwang, U. Tran, L. Eckmann, R. L. Gallo, and S. L. Reed. 2012. Entamoeba histolytica induces intestinal cathelicidins but is resistant to cathelicidin-mediated killing. Infection and immunity 80:143-149.
92. Bansal, D., R. Sehgal, Y. Chawla, R. C. Mahajan, and N. Malla. 2004. In vitro activity of antiamoebic drugs against clinical isolates of Entamoeba histolytica and Entamoeba dispar. Annals of clinical microbiology and antimicrobials 3:27.
93. Voogd, C. E. 1981. On the mutagenicity of nitroimidazoles. Mutat Res 86:243-277.
94. Salamanca-Pinzon, S. G., R. Camacho-Carranza, S. L. Hernandez-Ojeda, B. A. Frontana-Uribe, C. I. Espitia-Pinzon, and J. J. Espinosa-Aguirre. 2010. Correlation of the genotoxic activation and kinetic properties of Salmonella enterica serovar Typhimurium nitroreductases SnrA and cnr with the redox potentials of nitroaromatic compounds and quinones. Mutagenesis 25:249-255.
95. Fenech, M. 2007. Cytokinesis-block micronucleus cytome assay. Nature protocols 2:1084-1104.
96. Kirsch-Volders, M., I. Decordier, A. Elhajouji, G. Plas, M. J. Aardema, and M. Fenech. 2011. In vitro genotoxicity testing using the micronucleus assay in cell lines, human lymphocytes and 3D human skin models. Mutagenesis 26:177-184.
97. Diaz, D., A. Scott, P. Carmichael, W. Shi, and C. Costales. 2007. Evaluation of an automated in vitro micronucleus assay in CHO-K1 cells. Mutat Res 630:1-13.
98. Mortelmans, K., and E. Zeiger. 2000. The Ames Salmonella/microsome mutagenicity assay. Mutat Res 455:29-60.
99. Resende, F. A., W. Vilegas, L. C. Dos Santos, and E. A. Varanda. 2012. Mutagenicity of flavonoids assayed by bacterial reverse mutation (Ames) test. Molecules 17:5255-5268.
100. Payne, D. J., M. N. Gwynn, D. J. Holmes, and D. L. Pompliano. 2007. Drugs for bad bugs: confronting the challenges of antibacterial discovery. Nature reviews. Drug discovery 6:29-40.
101. Solaymani-Mohammadi, S., and S. M. Singer. 2011. Host immunity and pathogen strain contribute to intestinal disaccharidase impairment following gut infection. J Immunol 187:3769-3775.
102. Bouza, M., I. Maciqucs, D. Torres, and F. A. Nunez. 2000. Giardia lamblia in Mongolian gerbils: characteristics of infection using different human isolates. Exp Parasitol 96:43-46.
103. Buret, A., J. A. Hardin, M. E. Olson, and D. G. Gall. 1992. Pathophysiology of small intestinal malabsorption in gerbils infected with Giardia lamblia. Gastroenterology 103:506-513.
104. Deselliers, L. P., D. T. Tan, R. B. Scott, and M. E. Olson. 1997. Effects of Giardia lamblia infection on gastrointestinal transit and contractility in Mongolian gerbils. Digestive diseases and sciences 42:2411-2419.
105. Troeger, H., H. J. Epple, T. Schneider, U. Wahnschaffe, R. Ullrich, G. D. Burchard, T. Jelinek, M. Zeitz, M. Fromm, and J. D. Schulzke. 2007. Effect of chronic Giardia lamblia infection on epithelial transport and barrier function in human duodenum. Gut56:328-335.
106. Agueros, M., S. Espuelas, I. Esparza, P. Calleja, I. Penuelas, G. Ponchel, and J. M. Irache. 2011. Cyclodextrin-poly(anhydride) nanoparticles as new vehicles for oral drug delivery. Expert opinion on drug delivery 8:721-734.
107. Svenson, S. 2009. Dendrimers as versatile platform in drug delivery applications. European journal of pharmaceutics and biopharmaceutics: official journal of Arbeitsgemeinschafi fur Pharmazeutische Verfahrenstechnik e. V 71:445-462.
108. Whiting, M., J. C. Tripp, Y. C. Lin, W. Lindstrom, A. J. Olson, J. H. Elder, K. B. Sharpless, and V. V. Fokin. 2006. Rapid discovery and structure-activity profiling of new inhibitors of human immunodeficiency virus type 1 protease enabled by the copper(I)-catalyzed synthesis of 1,2,3-triazoles and their further functionalization. J Med Chem 49:7697-7710.
109. Rivkin, A., and S. Gim. 2011. Rifaximin: new therapeutic indication and future directions. Clinical therapeutics 33:812-827.
110. Ray, S., P. B. Madrid, P. Catz, S. E. LeValley, M. J. Furniss, L. L. Rausch, R. K. Guy, J. L. DeRisi, L. V. Iyer, C. E. Green, and J. C. Mirsalis. 2010. Development of a new generation of 4-aminoquinoline antimalarial compounds using predictive pharmacokinetic and toxicology models. J Med Chem 53:3685-3695.
111. Zhang, Y., J. A. Clark, M. C. Connelly, F. Zhu, J. Min, W. A. Guiguemde, A. Pradhan, L. Iyer, A. Furimsky, J. Gow, T. Parman, F. El Mazouni, M. A. Phillips, D. E. Kyle, J. Mirsalis, and R. K. Guy. 2012. Lead optimization of 3-carboxyl-4(1H)-quinolones to deliver orally bioavailable antimalarials. J Med Chem 55:4205-4219.
112. Rhomberg, L. R., K. Baetcke, J. Blancato, J. Bus, S. Cohen, R. Conolly, R. Dixit, J. Doe, K. Ekelman, P. Fenner-Crisp, P. Harvey, D. Hattis, A. Jacobs, D. Jacobson-Kram, T. Lewandowski, R. Liteplo, O. Pelkonen, J. Rice, D. Somers, A. Turturro, W. West, and S. Olin. 2007. Issues in the design and interpretation of chronic toxicity and carcinogenicity studies in rodents: approaches to dose selection. Critical reviews in toxicology 37:729-837.
113. Tweats, D. J., D. Blakey, R. H. Heflich, A. Jacobs, S. D. Jacobsen, T. Morita, T. Nohmi, M. R. O'Donovan, Y. F. Sasaki, T. Sofuni, and R. Tice. 2007. Report of the IWGT working group on strategy/interpretation for regulatory in vivo tests II. Identification of in vivo-only positive compounds in the bone marrow micronucleus test. Mutat Res 627:92-105.
114. Beena, Kumar N, Rohilla R K, Roy N, & Rawat D S (2009) Synthesis and antibacterial activity evaluation of metronidazole-triazole conjugates. Bioorg. Med. Chem. Lett. 19(5):1396-1398.
115. Hay M P, Lee H H, Wilson W R, Roberts P B, & Denny W A (1995) Hypoxia-selective antitumor agents. 10. bis(nitroimidazoles) and related Bis(nitroheterocycles): development of derivatives with higher rates of metabolic activation under hypoxia and improved aqueous solubility. J. Med. Chem. 38(11):1928-1941.
116. Chauviere G, et al. (2003) Synthesis and biological activity of nitro heterocycles analogous to megazol, a trypanocidal lead. J. Med. Chem. 46(3):427-440.
117. Adebayo ATOM, Bowman W R, & Salt W G (1987) Radical-Nucleophilic Substitution (Srn1) Reactions 0.5. Anions of Nitroimidazoles in Srn1 and Oxidative Addition-Reactions. Journal of the Chemical Society-Perkin Transactions 1 (12):2819-2827.
118. Salgado-Zamora H, Campos E, Jimenez R, Sanchez-Pavon E, & Cervantes H (1999) A convenient approach to the synthesis of the imidazo[5,1-b]oxazole ring system. Heterocycles 50(2):1081-1090.
119. Kulkarni S, Grimmett M R, Hanton L R, & Simpson J (1987) Nucleophilic Displacements of Imidazoles 0.1. Oxygen, Nitrogen and Carbon Nucleophiles. Aust. J. Chem. 40(8):1399-1413.
120. Kerins F & O'Shea D F (2002) Generation of substituted styrenes via Suzuki cross-coupling of aryl halides with 2,4,6-trivinylcyclotriboroxane. J. Org. Chem. 67(14):4968-4971.
121. Li C, et al. (2007) Unusual fluorescence enhancement of a new carbazolyldiacetylene bound to gold nanoparticles. Langmuir 23(12):6754-6760.
122. Abele R, et al. (2002) Synthesis and Cytotoxicity of 3-(Hetarylthio)-1-propynyl(trimethyl)silanes. Chemistry of Heterocyclic Compounds 38(7):867-872.
123. Toma T, Kita Y, & Fukuyama T (Total synthesis of (+)-manzamine A. J. Am. Chem. Soc. 132(30):10233-10235.
124. Hashmi A S, Weyrauch J P, Frey W, & Bats J W (2004) Gold catalysis: mild conditions for the synthesis of oxazoles from N-propargylcarboxamides and mechanistic aspects. Org Lett 6(23):4391-4394.
125. Zhou Y G, Yang P Y, & Han X W (2005) Synthesis and highly enantioselective hydrogenation of exocyclic enamides: (Z)-3-arylidene-4-acetyl-3,4-dihydro-2H-1,4-benzoxazines. J. Org. Chem. 70(5):1679-1683.
126. Kosiova I, Janicova A, & Kois P (2006) Synthesis of coumarin or ferrocene labeled nucleosides via Staudinger ligation. Beilstein J Org Chem 2:23.
127. Majumdar K C, Ghosh M, & Jana M (2002) Regioselective synthesis of thieno[2,3-b]quinolin-4(9H)-ones: Occurrence of thermal [1,3] sigmatropic rearrangement. Synthesis-Stuttgart (5):669-673.
128. Whiting M, et al. (2006) Rapid discovery and structure-activity profiling of new inhibitors of human immunodeficiency virus type 1 protease enabled by the copper(I)-catalyzed synthesis of 1,2,3-triazoles and their further functionalization. J. Med. Chem. 49(26):7697-7710.
129. Zheng H, et al. (2005) Design, synthesis, and evaluation of new bifunctional iron-chelators as potential agents for neuroprotection in Alzheimer's, Parkinson's, and other neurodegenerative diseases. Bioorg. Med. Chem. 13(3):773-783.
130. Chandra T, Pink M, & Zaleski J M (2001) Macrocyclic metalloenediynes of Cu(II) and Zn(II): a thermal reactivity comparison. Inorg. Chem. 40(23):5878-5885.
131. Iddon B, Khan N, & Lim B L (1987) Azoles 0.7. A Convenient Synthesis of Thieno[2,3-D]Imidazoles. Journal of the Chemical Society-Perkin Transactions 1 (7): 1457-1463.
132. Adriaenssens L, et al. (2009) Helquats: a facile, modular, scalable route to new helical dications. Chemistry 15(5):1072-1076.
133. Melo TMVDP, et al. (1999) Intramolecular dipolar cycloaddition reaction of 5H,7H-thiazolo[3,4-c]oxazol-4-ium-1-olates: synthesis of chiral 1H-pyrrolo[1,2-c]thiazole derivatives (vol 1, pg 1219, 1999). Journal of the Chemical Society-Perkin Transactions 1 (13): 1897-1897.
134. Gilchrist T L, Wasson R C, King F D, & Wootton G (1987) Formation of Pyridazino[6,1-C][1,4]Oxazin-8(7 h)-Ones by Intramolecular Cycloaddition of Azoalkenes. Journal of the Chemical Society-Perkin Transactions 1 (11):2517-2522.
135. Kobayashi K, Hashimoto K, Fukamachi S, & Konishi H (2008) Synthesis of (Z)-2-[(Z)-3-alkylideneisobenzofuran-1(3H)-ylidene]acetic acid derivatives by sequential coupling-cyclization between 3-(2-iodophenyl)-3-oxopropanoic acid derivatives and terminal alkynes. Synthesis-Stuttgart (7):1094-1098.
136. Cami G E, et al. (2008) Synthesis and characterization of a Cu(II) complex of 2-benzylmercapto-5-methyl-1,3,4-thiadiazole (C10H10N2S2). J. Coord. Chem. 61(19):3122-3133.
137. Ramenda T, Kniess T, Bergmann R, Steinbach J, & Wuest F (2009) Radiolabelling of proteins with fluorine-18 via click chemistry. Chem Commun (Camb) (48):7521-7523.
138. Rigaut S, et al. (2005) Carbon-rich ruthenium complexes containing Bis(allenylidene) and mixed alkynyl-allenylidene bridges. European Journal of Inorganic Chemistry (3):447-460.
139. Mabrour M, Bougrin K, Benhida R, Loupy A, & Soufiaoui M (2007) An efficient one-step regiospecific synthesis of new isoxazolines and isoxazoles of N-substituted saccharin derivatives through solvent-free microwave-assisted [3+2] cycloaddition. Tetrahedron Lett. 48(3):443-447.
140. Vereshchagin L I, et al. (2006) Polynuclear nonfused tetrazole-, 1,3,4-oxadiazole-, and 1,2,3-triazole-containing systems. Russian Journal of Organic Chemistry 42(6):912-917.
141. Rodriguez-Franco M I, Dorronsoro I, Badia A, & Banos J E (2002) Synthesis of new 1-(but-2-ynyl)pyrazoles: containing a pyrrolidine or diethylamine moiety and their muscarinic properties. Arch. Pharm. (Weinheim). 335(7):339-346.
142. Lipshutz B H, Chung D W, Rich B, & Corral R (2006) Simplification of the Mitsunobu reaction. Di-p-chlorobenzyl azodicarboxylate: a new azodicarboxylate. Org Lett 8(22):5069-5072.
143. Yang L X & Hofer K G (1996) Synthesis of 2-methyl-5-nitroimidazol-1-yl-acetaldehyde. Synth. Commun. 26(19):3653-3657.
144. Guillon R, et al. (2009) Design of new antifungal agents: synthesis and evaluation of 1-[(1H-indol-5-ylmethyl)amino]-2-phenyl-3-(1H-1,2,4-triazol-1-yl)propan-2-ols. Bioorg. Med. Chem. Lett. 19(20):5833-5836.
145. Townson S M, Laqua H, Uperoft P, Boreham P F L, & Uperoft J A (1992) Induction of Metronidazole and Furazolidone Resistance in Giardia. Trans. R. Soc. Trop. Med. Hyg. 86(5):521-522.
146. Boreham P F L, Phillips R E, & Shepherd R W (1988) Altered Uptake of Metronidazole Invitro by Stocks of Giardia-Intestinalis with Different Drug Sensitivities. Trans. R. Soc. Trop. Med. Hyg. 82(1):104-106.
147. Valdez C A, et al. (2009) Synthesis and Electrochemistry of 2-Ethenyl and 2-Ethanyl Derivatives of 5-Nitroimidazole and Antimicrobial Activity against Giardia lamblia. J. Med. Chem. 52(13):4038-4053.
148. Brown D M, Uperoft J A, Dodd F I N, Chen N, & Uperoft P (1999) Alternative 2-keto acid oxidoreductase activities in Trichomonas vaginalis. Mol. Biochem. Parasitol. 98(2):203-214.
149. Goldman L M, Uperoft J A, Workowski K, & Rapkin A (2009) Treatment of metronidazole-resistant Trichomonas vaginalis. Sexual Health 6(4):345-347
150. Uperoft J A & Uperoft P (2001) Drug Susceptibility Testing of Anaerobic Protozoa. Antimicrob. Agents Chemother. 45(6):1810-1814.
151. Clark C G & Diamond L S (2002) Methods for cultivation of luminal parasitic protists of clinical importance. Clin. Microbiol. Rev. 15(3):329-+.
152. Kersulyte D, et al. (2000) Differences in genotypes of Helicobacter pylori from different human populations. J. Bacteriol. 182(11):3210-3218.
153. Jeong J Y, et al. (2001) Roles of FrxA and RdxA nitroreductases of Helicobacter pylori in susceptibility and resistance to metronidazole. J. Bacteriol. 183(17):5155-5162.
154. Tetko I V, et al. (2005) Virtual computational chemistry laboratory—design and description. J. Comput. Aided. Mol. Des. 19(6):453-463.
155. Hall M, et al. (2009) The WEKA Data Mining Software: An Update. SIGKDD Explorations 11(1).
156. OECD (2010) Test No. 487: In Vitro Mammalian Cell Micronucleus Test. <http://dx.doi.org/10.1787/9789264091016-en>.

LENGTHY TABLES
The patent application contains a lengthy table section. A copy of the table is available in electronic form from the USPTO web site (http://seqdata.uspto.gov/?pageRequest=docDetail&DocID=US20160244435A1). An electronic copy of the table will also be available from the USPTO upon request and payment of the fee set forth in 37 CFR 1.19(b)(3).

1. A compound having the structure of Formula (I) 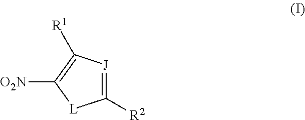
or pharmaceutically acceptable salts thereof, wherein
J is N or CR³;
is NR⁴, S, or O;
R¹ is hydrogen or —(CH₂)ₘ—Y—R⁵;
R² is hydrogen, —(CH₂)ₘ—Y—R⁵, —CH═CH—R⁵, —CHR^{a}—CHR^{b}—R⁵, —C═N—O—(CH₂)ₘ—R⁵, —NHC(O)—(CH₂)ₘ—R⁵, optionally substituted —S—C₁₋₆ alkyl, optionally substituted —O—C₁₋₆alkyl, optionally substituted C₁₋₆alkyl, optionally substituted C₂₋₁₀alkenyl, optionally substituted C₂₋₁₀alkynyl, optionally substituted C₃₋₁₀ cycloalkyl, optionally substituted 4-10 membered heterocyclyl, optionally substituted C₆₋₁₀ aryl, or optionally substituted 5-10 membered heteroaryl;
R³ is hydrogen, halogen, hydroxyl, or optionally substituted —C₁₋₆ alkyl;
R⁴ is hydrogen, C₁₋₆alkyl or —(CH₂)ₘ—Y—R⁵;
m is an integer between 0 to 5;
Y is optionally substituted heteroaryl;
each R⁵ is independently hydrogen, —(CH₂)ₙ-M, or —(CH₂)ₙ—Y′-M;
each M is independently C(O)M′, —C(O)O—C₁₋₄alkyl, —CH₂-M′, —S-M′, —NHC(O)-M′, —NHC(O)NH-M′, —O—C₆H₄—C(O)NH-M′, —O-M′, —O(CH₂)C(O)-M′, —N(CH₃)-M′, optionally substituted —S—C₁₋₆ alkyl, optionally substituted —O—C₁₋₆alkyl, optionally substituted C₁₋₆alkyl, optionally substituted C₂₋₁₀alkenyl, optionally substituted C₂₋₁₀ alkynyl, optionally substituted C₃₋₁₀ cycloalkyl, optionally substituted 4-10 membered heterocyclyl, optionally substituted C₆₋₁₀ aryl, or optionally substituted 5-13 membered heteroaryl;
each M′ is independently hydrogen, optionally substituted C₁₋₆alkyl, optionally substituted C₂₋₁₀alkenyl, optionally substituted C₂₋₁₀alkynyl, optionally substituted C₃₋₁₀ cycloalkyl, optionally substituted 4-10 membered heterocyclyl, optionally substituted C₆₋₁₀ aryl, or optionally substituted 5-13 membered heteroaryl;
each Y′ is independently optionally substituted 4-10 membered heterocyclyl or C₆₋₁₀ aryl;
n is an integer between 0 to 5;
R^{a} and R^{b} are each independently selected from —H, hydroxy, halogen, —C₁₋₄alkyl, —O—C₁₋₄alkyl, —O—C(O)—C₁₋₄alkyl;
with the proviso that the compound does not have the structure selected from the group consisting of

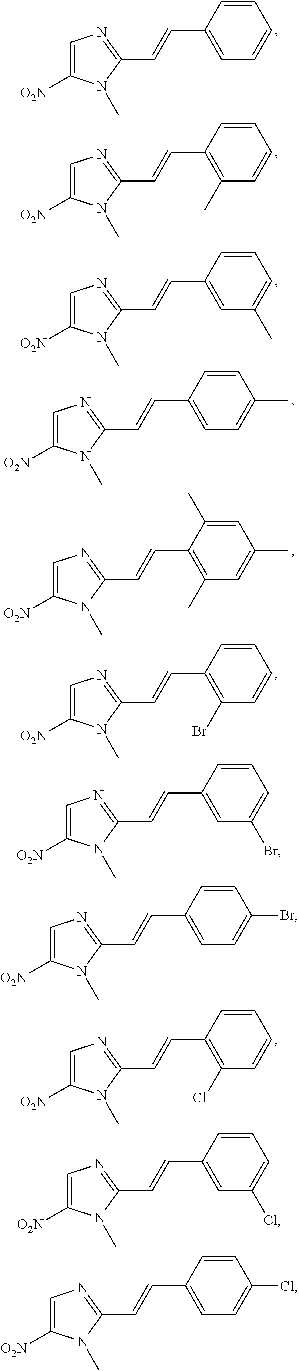

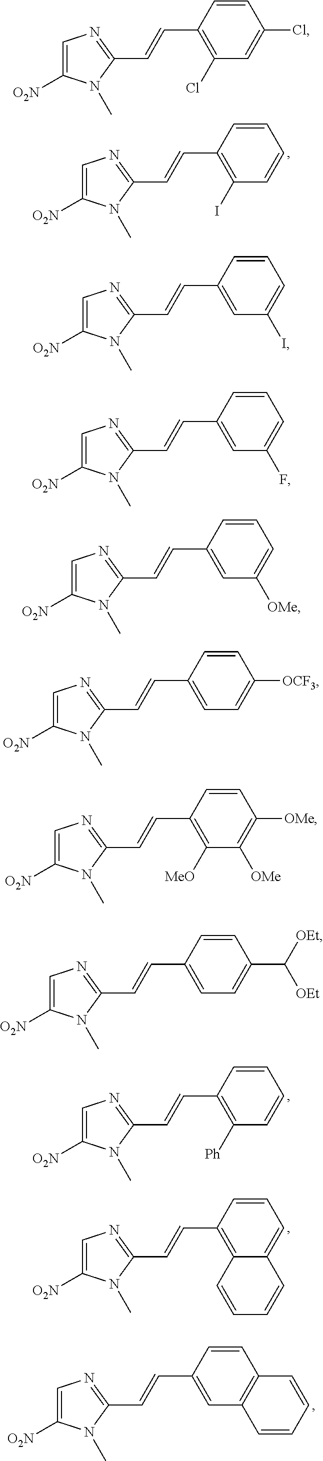

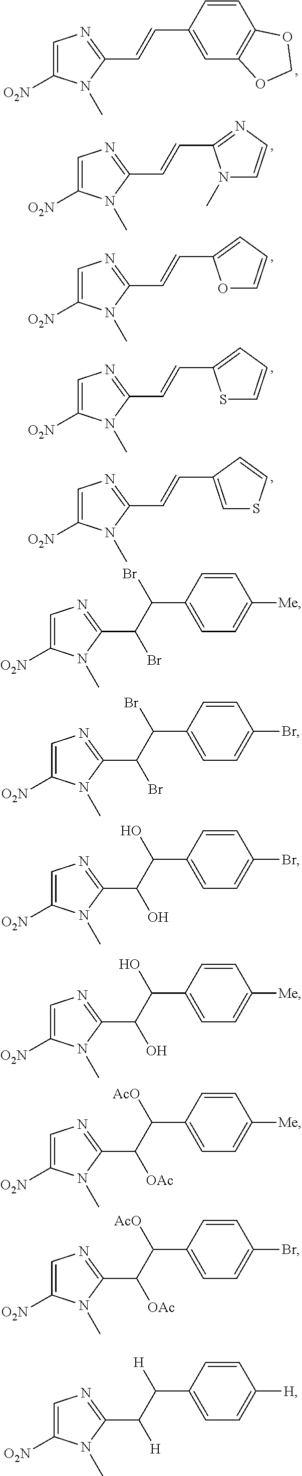

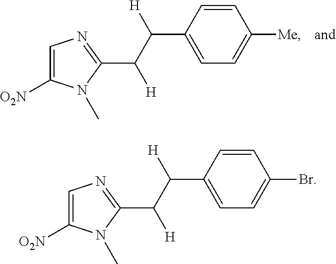 [SMILES: Cc1ccc(CCc2ncc([N+](=O)[O-])n2C)cc1 ; Cn1c([N+](=O)[O-])cnc1CCc1ccc(Br)cc1]

2. The compound of claim 1, with the proviso that when L is —N—CH₃ and J is N, R² is not —CH═CH—R^{e} or —CHX—XHY—R^{f}, wherein R^{e} is optionally substituted phenyl, naphthalenyl, thiophene, furan, 1,3-benzodioxol, or imidazolinylmethyl; and R^{f} is phenyl optionally substituted at the para position with methyl or bromine.

3. The compound of claim 1, wherein J is N and L is NR⁴.

4. The compound of claim 1, having the structure selected from the group consisting of 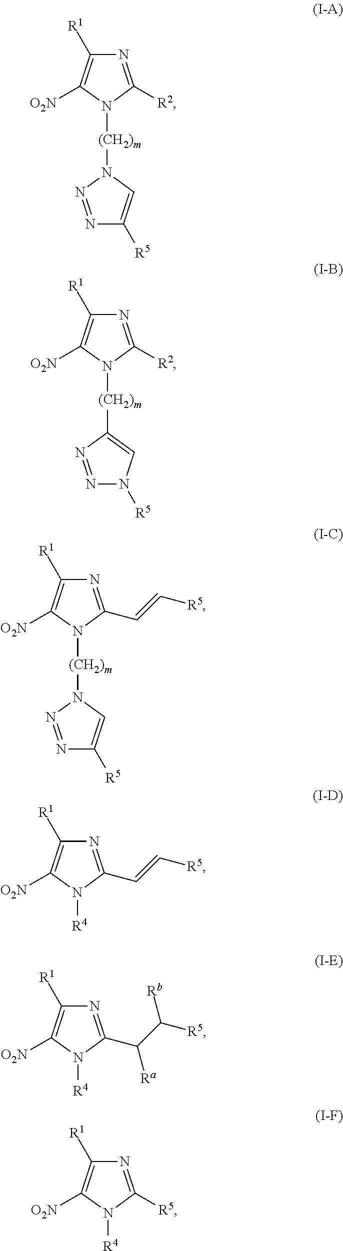 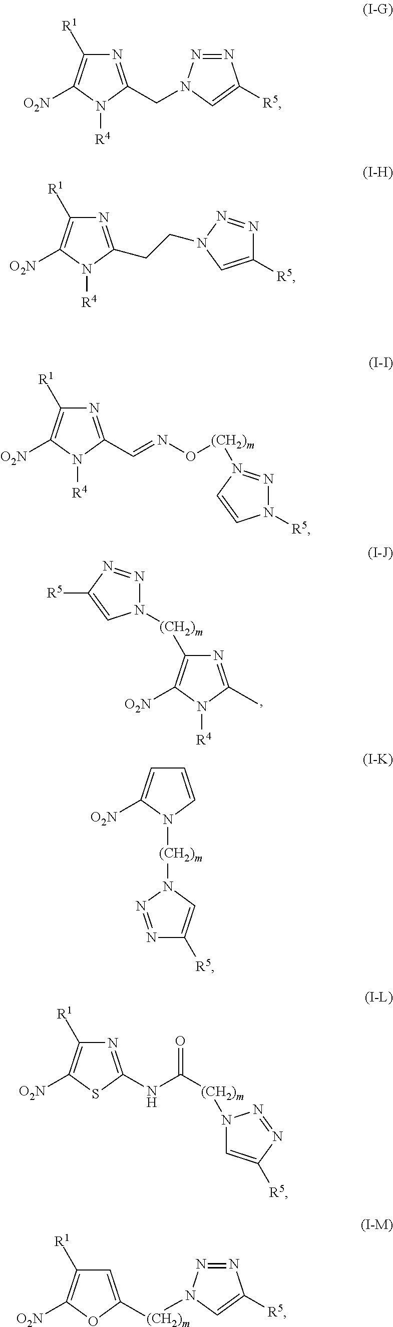
and
or pharmaceutically acceptable salts thereof.

5. The compound of claim 4, wherein the compound is selected from the group consisting of compounds A-101 to A-163 in Table S1a and A-201- to A-247 in Table S6a.

6. (canceled)

7. The compound of claim 4, wherein R² is C₁₋₆ alkyl.

8. (canceled)

9. (canceled)

10. The compound of claim 4, wherein the compound is selected from the group consisting of compounds C(1)-101 to C(1)-163 in Table S1e, C(2)-101 to C(2)-163 in Table S1e, C(1)-201- to C(1)-247 in Table S6e, and C(2)-201- to C(2)-247 in Table S6e.

11. (canceled)

12. (canceled)

13. (canceled)

14. (canceled)

15. (canceled)

16. The compound of claim 4, wherein the compound is selected from the group consisting, of compounds listed in Table S10.

17. (canceled)

18. The compound of claim 4, wherein the compound is selected from the group consisting of compounds G-101 to G-163 in Table S1b and 0-201- to G-247 in Table S6b.

19. (canceled)

20. The compound of claim 4, wherein the compound is selected from the group consisting of compounds H-101 to H-163 in Table S1c and H-201- to H-247 in Table S6c.

21. (canceled)

22. The compound of claim 4, wherein the compound is selected from the group consisting of compounds in Table S11.

23. (canceled)

24. The compound of claim 4, wherein the compound is selected from the group consisting of compounds J-101 to J-163 in Table S1d and J-201- to J-247 in Table S6d.

25. The compound of claim 4, wherein R⁴ is hydrogen or C₁₋₄alkyl.

26. (canceled)

27. (canceled)

28. (canceled)

29. The compound of claim 4, wherein the compound is selected from the group consisting of compounds in Table S12.

30. (canceled)

31. The compound of claim 4, wherein the compound is selected from the group consisting of compounds in Table S13.

32. The compound of claim 1, wherein R¹ is hydrogen.

33. (canceled)

34. (canceled)

35. (canceled)

36. The compound of claim 1, wherein R⁵ is selected from the group consisting of

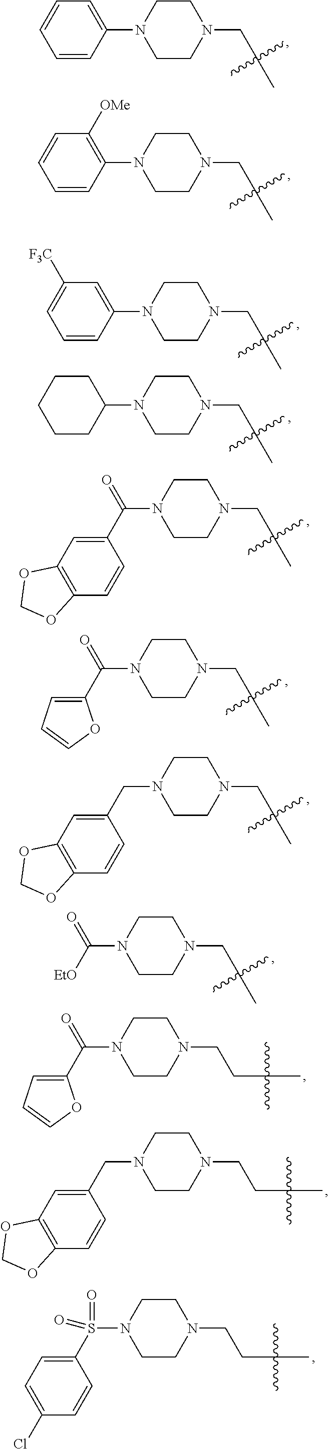

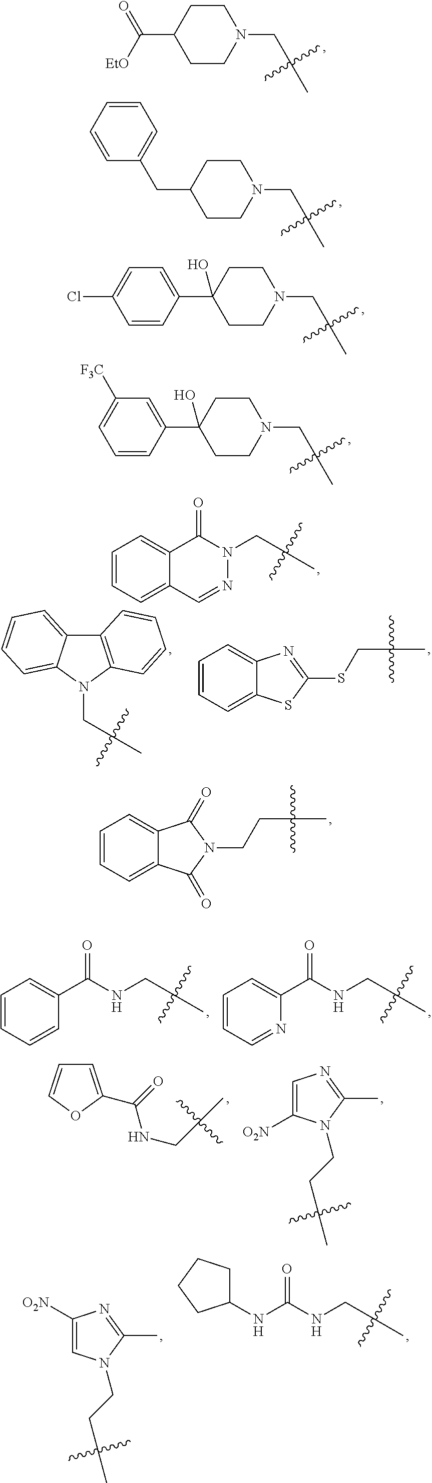

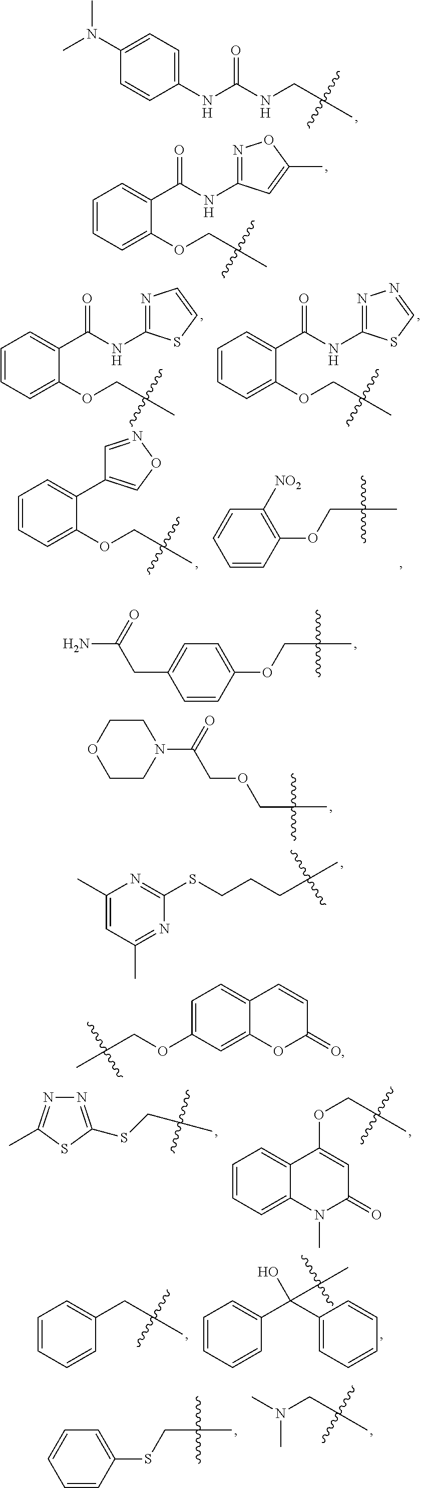

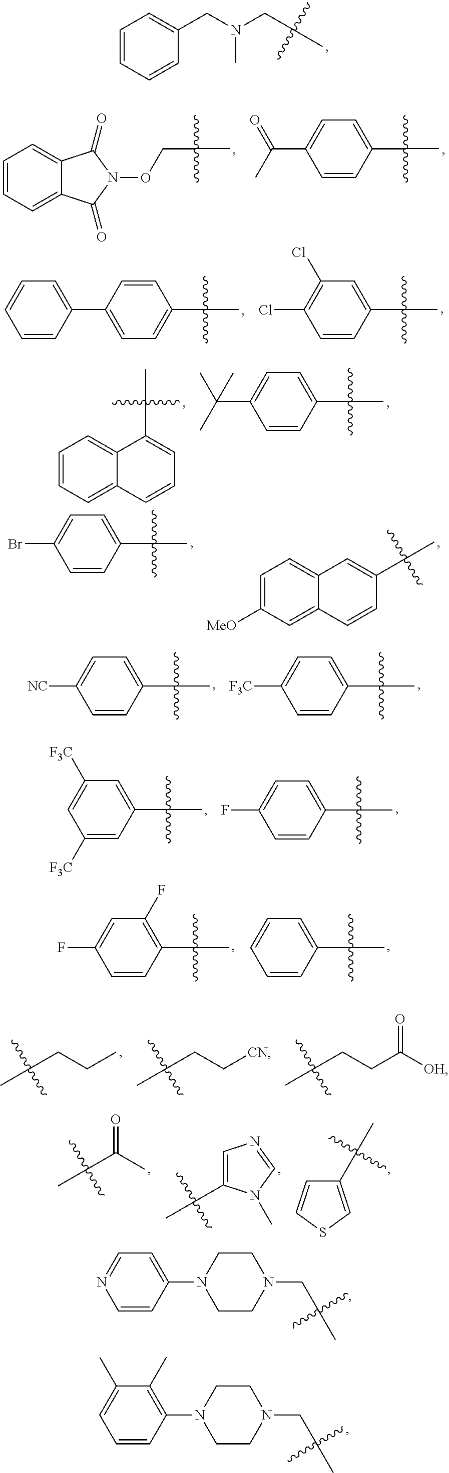

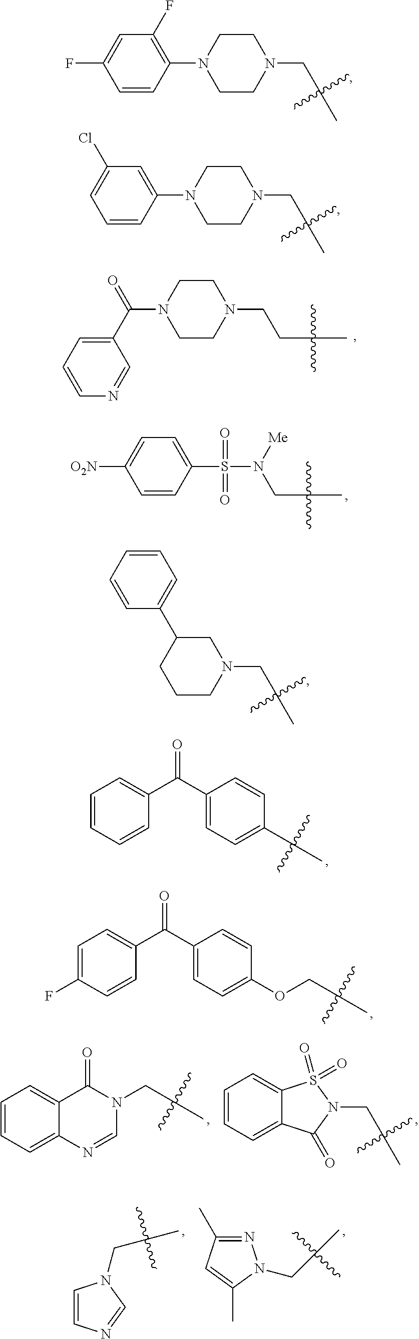

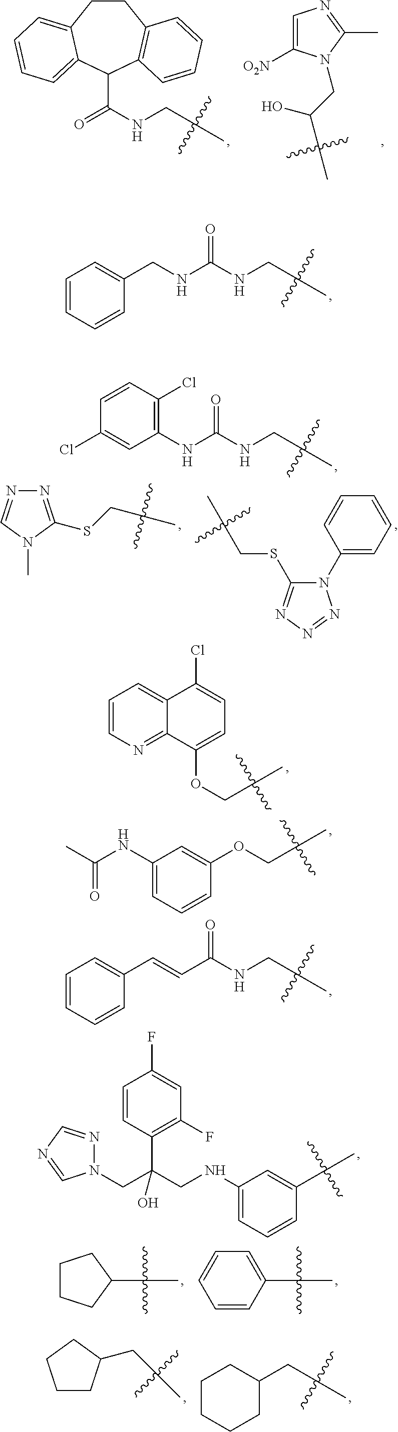

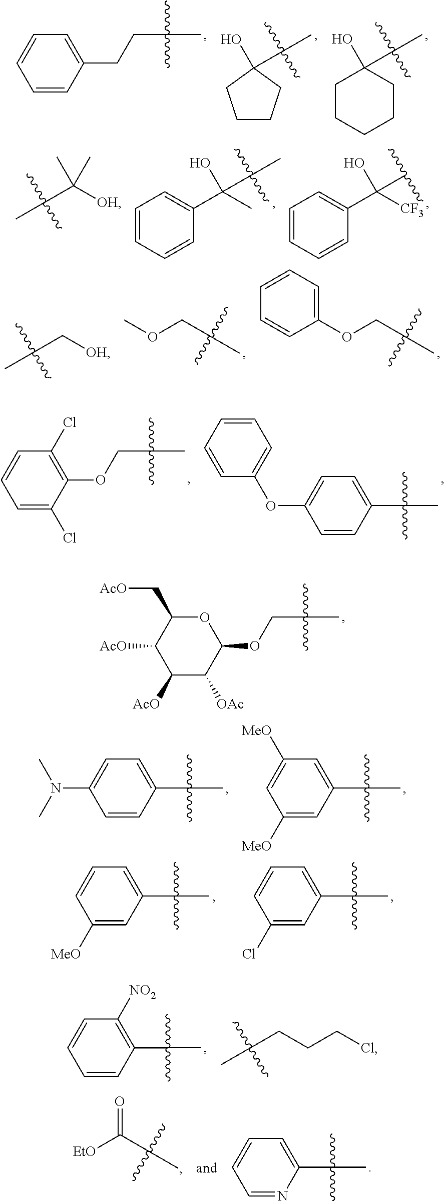

37. (canceled)

38. (canceled)

39. (canceled)

40. A pharmaceutical composition comprising a therapeutically effective amount of a compound of claim 1 and a pharmaceutically acceptable excipient.

41. (canceled)

42. (canceled)

43. A method of ameliorating a Trichomononas vaginalis infection, Giardia lamblia infection, Entamoeba histolytica infection, or a gram-negative bacterial infection selected from the group consisting of Helicobacter pylori, Clostridium difficile and Bacteroides fragilis, comprising administering to a subject in need thereof a therapeutically effective amount a compound of claim 1.

44. The method of claim 43, further comprising administering to the subject an additional medicament, wherein the additional medicament is selected from an antibacterial agent, an antifungal agent, an antiviral agent, an anti-inflammatory agent, or an antiallergic agent.

45-61. (canceled)
